(19) United States
(12) Patent Application Publication
(10) Pub. No.: US 2004/0002060 A1
(43) Pub. Date: Jan. 1, 2004

(54) Fiber shaft modifications for efficient targeting

(75) Inventors: Michael Kaleko, Rockville, MD (US); Glen R. Nemerow, Encinitas, CA (US); Theodore Smith, Ijamsville, MD (US); Susan C. Stevenson, Frederick, MD (US)

Correspondence Address: HELLER EHRMAN WHITE & MCAULIFFE LLP, 4350 LA JOLLA VILLAGE DRIVE, 7TH FLOOR, SAN DIEGO, CA, 92122-1246, US

(73) Assignees: Novartis AG; The Scripps Research Institute

(21) Appl. No.: 10/351,890

(22) Filed: Jan. 24, 2003

Related U.S. Application Data
(60) Provisional application No. 60/350,388, filed on Jan. 24, 2002.
(60) Provisional application No. 60/391,967, filed on Jun. 26, 2002.

(51) Int. Cl. C12Q 1/70; C07H 21/04; C12N 7/00; C12N 15/861; C07H 21/02; C07K 14/005

(52) U.S. Cl. 435/5; 435/69.3; 435/235.1; 435/320.1; 435/456; 435/325; 530/350; 536/23.72

(57) ABSTRACT

Provided are adenoviral vectors and the production of such vectors. In particular, fiber shaft modifications for efficient targeting of adenoviral vectors are provided. The fiber shaft modifications can be combined with other modifications, such as fiber knob and/or penton modifications, to produce fully ablated (detargeted) adenoviral vectors. A scale-up method for the propagation of detargeted adenoviral vectors is also provided.

### RELATED APPLICATIONS

Benefit of priority is claimed under 35 U.S.C. §119(e) to U.S. provisional application Serial No. 60/350,388, filed Jan. 24, 2002, entitled “FIBER SHAFT MODIFICATIONS FOR EFFICIENT TARGETING,” to Stevenson, Susan C., Kaleko, Michael, Smith, Theodore and Nemerow, Glen R., and to U.S. provisional application Serial No. 60/391,967, filed Jun. 26, 2002, entitled “FIBER SHAFT MODIFICATIONS FOR EFFICIENT TARGETING,” to Stevenson, Susan C., Kaleko, Michael, Smith, Theodore and Nemerow, Glen R. This application is also related to International PCT application No. (attorney docket number 22908-1236PC), filed the same day herewith, entitled “FIBER SHAFT MODIFICATIONS FOR EFFICIENT TARGETING,” to Stevenson, Susan C., Kaleko, Michael, Smith, Theodore and Nemerow, Glen R. The subject matter of each of these applications is incorporated by reference herein.

### FIELD OF INVENTION

The present invention generally relates to the field of adenoviral vectors and the production of such vectors. In particular, detargeted adenoviral vectors are provided.

### BACKGROUND

Most, if not all, adenoviral vector-mediated gene therapy strategies aim to transduce a specific tissue, such as a tumor or an organ. Systemic delivery will require ablation of the normal virus tropism as well as addition of new specificities. Multiple interactions between adenoviral particles and the host cell are required to promote efficient cell entry (Nemerow (2000) Virology 274:1-4). An adenovirus entry pathway is believed to involve two separate cell surface events. First, a high affinity interaction between the adenoviral fiber knob and coxsackie-adenovirus receptor (CAR) mediates the attachment of the adenovirus particle to the cell surface. A subsequent association of penton with the cell surface integrins αᵥβ₃ and αᵥβ₅, which act as co-receptors, potentiates virus internalization. There are a plurality of adenoviral fiber receptors, which interact with the group B (e.g., Ad3) and group C (e.g., Ad5) adenoviruses. Both of these groups of adenoviruses appear to require interaction with integrins for internalization. CAR ablation, however, does not change biodistribution and toxicity of adenoviral vectors in vivo (Alemany et al. (2001) Gene Therapy 8:1347-1353; U.S. patent application Ser. No. 09/870,203, filed May 30, 2001, and published as U.S. Published application No. 20020137213). Thus, the role of CAR interaction for in vivo gene transfer is not clear. Recently published studies have described conflicting results (Alemany et al. (2001) Gene Therapy 8:1347-1353; Leissner et al. (2001) Gene Therapy 8:49-57; Einfeld et al. (2001) J. Virology 75:11284-11291). For example, it has been shown that vectors containing an S408E mutation in the Ad5 fiber AB loop yield efficient liver transduction in mice, despite having greatly reduced transduction efficiencies on cells in culture (see, Leissner et al. (2001) Gene Therapy 8:49-57). In contrast, vectors containing a more extensive fiber AB loop mutation showed a 10-fold reduction in liver gene expression (see, Einfeld et al. (2001) J. Virology 75:11284-11291).

A doubly ablated adenovirus has been prepared by modifying the CAR binding region in the fiber loop and the integrin binding region in the penton base (Einfeld et al. (2001) J. Virology 75:11284-11291). This doubly ablated adenovirus, lacking CAR and integrin interactions, was reported not only to lack in vitro transduction of various cell types but also to lack in vivo transduction of liver cells. Specifically, the doubly ablated adenovirus was reported to have a 700 fold reduction in liver transduction when compared to the non-ablated adenovirus. These results, however, were not reproduced by others.

For many applications, the most clinically useful adenoviral vector would be deliverable systemically, such as into a peripheral vein, and would be targeted to a desired location in the body, and would not have undesirable side effects resulting from targeting to other locations. In vivo adenoviral vector targeting is a major goal in gene therapy and a significant effort has been focused on developing strategies to achieve this goal. Successful targeting strategies would direct the entire vector dose to the appropriate site and would be likely to improve the safety profile of the vector by permitting the use of lower, less toxic vector doses, which potentially also can be less immunogenic. Thus, there is a need to develop adenoviruses which are fully detargeted in vivo for use as a base vector for producing redirected adenoviruses.

Therefore, among the objects herein, it is an object herein to provide fully detargeted adenoviral vectors, methods for preparation thereof, and uses thereof.

### SUMMARY

Detargeted and fully detargeted adenoviral particles, adenovirus vectors from which such particles are produced, methods for preparation of the vectors and particles and uses of the vectors and particles are provided. Provided and described are capsid modifications, such as fiber shaft modifications, and the resuling proteins that, when expressed on adenoviral particles provide for detargeting of adenoviral vectors. The capsid modifications, such as the fiber shaft modifications, can be combined with other modifications, such as fiber knob and/or penton modifications, to produce fully ablated (detargeted) adenoviral particles.

Thus, adenoviral vectors and adenovirual particles whose native tropisms are ablated through a modification or modifications of capsid proteins, particularly a fiber shaft region, are provided.

Thus, provided are capsid mutiations, including fiber shaft modifications, that ablate binding to particular receptors, thereby permitting efficient targeting of adenoviral vectors that contain capsids with such modifications. For example, adenoviral vectors in which the fiber shaft's interaction with HSP is ablated (reduced or substantially eliminated), particuarly in vivo, are provided. These fiber shaft modifications can be combined with other modifications, such as fiber knob and/or penton modifications, to produce fully ablated (detargeted) adenoviral vectors. Also provided are retargeted vectors and particles that include a ligand or ligands to provide for targeting of the detargeted vectors and particles to selected cells and/or tissues. Retargeting can be effected, for example, by manipulating the fiber protein to redirect the receptor specificity to a particular cell type.

Also provided are nucleic acids encoding the modified fiber proteins and also modified penton proteins. Also provided are nucleic acids encoding the modified fiber shaft protein that has ablated HSP binding and combinations thereof with other modified fiber regions or other proteins, such as a modified fiber knob region and/or the modified penton protein. The nucleic acids also can contain heterologous nucleic acid sequences, such as promoters or nucleic acid sequences encoding polypeptides. The viral particles that express fibers containing such shaft modifications and other modifications are also provided.

Also provided are methods for making and using the adenoviral particles that express the modified fibers and combinations of modified fibers and modified penton. With the fiber shaft modifications, particularly in combination with the fiber knob modifications and the penton modifications, the adenovirus particles are ablated for binding to their natural cellular receptor(s), i.e., they are detargeted. They can then be “retargeted” to a specific cell type through the addition of a ligand to the virus capsid, which causes the virus to bind to and infect such cell. The ligand can be added, for example, through genetic modification of a capsid protein gene.

Also provided is a method for reducing liver toxicity in adeno-viral-mediated therapy. In contrast to the results of Einfeld et al. (Einfeld et al. (2001) J. Virology 75:11284-11291), it is shown herein that a doubly ablated adenovirus, lacking CAR and integrin interactions, is capable of in vivo liver transduction. It is shown herein that ablation of liver transduction requires further and/or alternative modification(s). The method for reducing liver toxicity in adenoviral-mediated therapy includes modifying an adenoviral vector to ablate native tropism to liver cells in vivo. Such vector can be administered to a subject. The modifications include the modifications described herein.

The nucleic acids, proteins, adenoviral particles and adenoviral vectors have a variety of uses. These include in vivo and in vitro uses to target nucleic acid to particular cells and tissues, for therapeutic purposes, including gene therapy, and also for the identification and study of cell surface receptors and identification of modes of interaction of viruses with cells.

In particular, adenoviral fiber shaft modifications that ablate viral interaction with HSP (Heparin Sulfate Proteoglycans; also referred to as heparin sulfate glycosaminoglycans) are provided. These modifications include mutations of individual amino acids in the fiber shaft that interact with HSP or mutations of amino acids in the fiber shaft that modify the ability of the HSP binding motif to interact with HSP. Adenoviral fiber shaft modifications also include replacements of fiber shafts using fiber shafts of adenoviruses, such as, for example, Ad3, Ad35 and Ad41 short fiber shaft, that do not contain HSP binding sites.

Also provided are adenoviral fiber shaft modifications that alter, particularly ablate viral interaction with HSP, as described above, in combination with fiber knob modifications that ablate viral interaction with CAR. The fiber knob modifications include: (a) mutations of individual amino acids in the fiber loop that interact with CAR, such as, for example, AB or CD loop modifications; (b) mutations of individual amino acids in the fiber loop that modify the ability of the CAR binding motif to interact with CAR; and (c) replacements of fiber knobs using adenoviruses that do not interact with CAR, such as, for example, Ad3 fiber knob, Ad41 short fiber knob, or Ad35 fiber knob.

Also provided are adenoviral fiber shaft modifications as described above in combination with penton modifications that ablate viral interaction with aᵥ integrins. The penton modifications include: (a) mutations of individual amino acids that interact with αᵥ integrins; (b) mutations of individual amino acids that modify the ability of the αᵥ integrin binding motif to interact with the αᵥ integrins; and (c) replacement of penton proteins using penton proteins from adenoviruses that do not interact with the αᵥ integrins.

Also provided are adenoviral fiber shaft modifications as described above in combination with fiber knob modifications as described above and penton modifications as described above.

Also provided is a scale-up method for the propagation of detargeted adenoviral vectors. The method uses polycations and/or bifunctional reagents, which when added to tissue culture medium results in entry of adenoviral particles into the producer cells.

Provided aer recombinant viral particles that contain a modified capid protein whereby binding to heparin sulfate proteoglycans (HSP) is reduced or eliminated compared to particles that contain unmodified capsid proteins. The modified capsid proteins include fibers proteins with modified shafts such that binding to HSP is reduced or eliminated.

Among the particular embodiments the following are provided. Provided are adenovirus capsid proteins that are modified to alter, typically reduce or eliminate, binding to or interaction with in vivo and/or in vitro to heparin sulfate proteoglycan (HSP). HSPs are expressed on various cells, including hepatocytes. It is shown herein that HSPs provide for or participate in transduction of cells, such as liver cells. Since it can be desirable to eliminate or reduce such transduction, the modifications of the capsid proteins, such as fiber proteins, permit detargeting of particles that express such proteins from such cells.

Thus provided are modified adenovirus fiber proteins that include a mutation, such as an insertion, deletion, change, replacement of amino acids or combinations thereof, whereby binding to or interaction with heparin sulfate proteoglycan (HSP) is altered. In particular, the he binding of the modified fiber protein is eliminated or reduced compared to the unmodified protein. Exemplary of these mutations are mutations in the shaft of a fiber, where the shaft also can include the tail. The mutations can reduce or alter the affinity of the fiber protein for HSP is reduced at least by 2-fold, 5-fold, 10-fold, 100-fold or more, including substantially eliminating it.

As provided herein, fibers from adenoviruses that interact with HSP can include a motif, such as BBXB or BBBXXB, wherethe B is a basic amino acid and X is any amino acid, particularly the consensus sequence KKTK in Ad5 and Ad2. Thus, provided are fibers in which the motif is altered to eliminate or reduce interaction with HSP.

Also provided are modified fiber protein of claim 1 that are chimeras in which the fiber shaft (or fiber shaft and tail) are derived from a fiber, such as Ad3, Ad35, Ad7, Ad11, Ad16, Ad21, Ad34, Ad40, Ad41 or Ad46 fiber, that does not interact with HSP and combined with fiber that does interact, such as Ad5 or Ad2 fiber, to produce a complete fiber whose binding to HSP is reduced or eliminated.

All of the modified capsids proteins provided herein also can include one or more further modifications that reduce or eliminate interaction of the resulting fiber with one or more cell surface proteins, such as but not limited to, CAR and αᵥ integrin or other receptor to which a particular native fiber binds, in addition to HSP. These modifications include, but are not limited to, modification to fiber that reduces or eliminates CAR binding and modification to penton that reduces or eliminate αᵥ integrin binding. The mutations can be in the fiber knob, shaft, tail and shaft, and also in penton.

Any and all of the modified capsid proteins provided herein can further include a ligand that binds to a particular receptor thereby endowing a fiber (or other capsid protein) with binding specificity or the ability to interact with such receptor. The ligand can be inserted into any suitable site in a capsid protein, such as an insertion or replacement. For example, fibers with ligands inserted into the knob region are exemplified. Any such ligand can be employed and a variety are exemplified herein.

A variety of modified capsid proteins are exemplified herein. These include, but are not limited to, fibers containing: the sequence of amino acids set forth in any of SEQ ID Nos. 52, 54, 56, 58, 62, 66, 70 and 72; or a sequence of amino acids having 60%, 70%, 80%, 90%, 95% or greater sequence identity with a sequence of amino acids set forth in any of SEQ ID Nos. 52, 54, 56, 58, 62, 66, 70 and 72; or a sequence of amino acids encoded by a sequence of nucleotides that hybridizes under conditions of high stringency along at least 70% of its length to a sequence of nucleotides that encodes a sequence of amino acids set forth in any of SEQ ID Nos. 52, 54, 56, 58, 62, 66, 70 and 72.

Nucleic acids encoding the capsid proteins, including the fibers are also provided. The nucleic acids can be provided as vectors, particularly as adenovirus vectors. Many adenoviral vectors are known and can be modified as needed in accord with the description herein. Adenoviral vectors include, but are not limited to, early generation adenoviral vectors, such as E1-deleted vectors, gutless adenoviral vectors and replication-conditional adenoviral vectors, such as oncolytic adenoviral vectors. The adenovirus vectors also can include heterologous nucleic acids that encode or provide products, such as therapeutic products. Any therapeutic product is contemplated and a variety are set forth herein as exemplary. Heterologous nucleic acid can encode a polypeptide or comprise or encode a regulatory sequence, such as a promoter or an RNA, including RNAi, small RNAs, other double-stranded RNAs, antisense RNA, and ribozymes. Promoters include, for example, constitutive and regulated promoters and tissue specific promoter, including tumor specific promoters. The promoter can be operably linked, for example, to a gene of an adenovirus essential for replication.

Cells containing the nucleic acid molecules and cells containing the vectors are also provided. Such cell include packaging cells. The cells can be prokaryotic or eukaryotic cells, including, mammalian cells, such a primate cells, including human cells.

Also provided are adenoviral particles that contain the modified capsid proteins provided herein. The particles have altered interaction or binding with HSP compared to particles that do not contain the modified capsid proteins. In addition to altered binding to HSP, which is typically reduced or eliminated binding, the particles can include further modifications, such as capsid proteins with altered interaction with other receptors as described above. In particular, the particles can have altered, typically reduced or eliminated, interaction with CAR, αᵥ integrin and/or other receptors. The mutation include mutations in the fiber knob, penton and hexon. Exemplary fiber know mutations are mutations in the AB loop or CD loop, such as KO1 or KO12, which are described herein. In addition, the particles can include additional ligands for retargeting to selected receptors. The adenoviral particles can be from any serotype and subgroup.

Methods for expressing heterologous nucleic acids in a cell are provided. In these methods an adenoviral vector provided herein is transduced into a cell to deliver the nucleic acid and/or encoded products. Transduction can be effected in vivo or in vitro or ex vivo, and can be for a variety of purposes including study of gene expression and genetic therapy. The cells can be prokaryotic cells, but typically are eukaryotic cells, including mammalian cells, such as primate, including human, cells. The cells can be of a specific type, such as a tumor cell or a cell in a particular tissue. The vectors can be oncolytic vector to effect killing of tumor cells.

Since the modified capsid proteins herein have reduced or eliminated binding to HSP, viral particles containing such proteins exhibit ablated binding to HSP in vitro and in vivo. Thus provided is a method of reducing transduction of cells that express HSP, such as hepatocytes in the liver, by modifying a capsid protein, such as fiber to eliminate or reduce interaction with or binding to HSP. Such reduction reduces or eliminates transduction of cells that express HSP, including liver cells.

Also provided are scale-up methods for the propagation of detargeted adenoviral particle, such as those provided herein. The method includes the steps of infecting or transducing a cell capable of replicating, maturing and packaging an adenoviral vector with a detargeted adenoviral vector in the presence of a reagent that results in entry of the adenoviral particle into the cell, such as a polycation and/or a bifunctional protein or other such reagent; and culturing the infected cell under conditions suitable for growth, spread and propagation of the adenoviral vector. The resulting adenoviral particles can be recovered. Polycations include, but are not limited to, hexadimethrine bromide, polyethylenimine, protamine sulfate and poly-L-lysine. Bifunctional proteins, include, but are not limited to, an anti-fiber antibody ligand fusion, an anti-fiber-Fab-FGF conjugate, an anti-penton-antibody ligand fusion, an anti-hexon antibody ligand fusion and a polylysine-peptide fusion. The ligand is selected to bind to a particular receptor.

The viral particles that express a modified capsids provided herein can be produced by this method. The modification include, for example, one or more mutations selected from among mutations that reduce or eliminate interactions with one or more of αᵥ integrins, coxsackie-adenovirus receptors (CAR) and heparin sulfate proteoglycans (HSP). Such mutations include, for example, PD1, KO1, KO12 and S*.

### BRIEF DESCRIPTION OF THE FIGURES

FIG. 1 is a plasmid map for pSKO1.

FIG. 2 is a plasmid map for pNDSQ3.1KO1.

FIGS. 3A-3C are plasmid maps of pAdmireRSVnBg.(FIG. 3A), pSQ1 FIG. 3B) and pSQ1KO12 (FIG. 3C)

FIG. 4 is a plasmid map for pSQ1PD1.

FIGS. 5A-5B are plasmid maps of pSQ1FKO1PD1 (FIG. 5A) and pSQ1KO12PD1 (FIG. 5B).

FIG. 6 shows in vitro transduction efficiency of A549 cells using adenoviral vectors containing fiber AB loop knob and/or penton, PD1 mutations. The following adenoviral vectors were used in these studies: Av1nBg, Av1nBgFKO1, referred to as FKO1, Av1nBgPD1, referred to as PD1, and Av1nBgFKO1PD1 that is referred to as FKO1PD1.

FIGS. 7A-7B shows in vivo adenoviral-mediated liver gene expression (FIG. 7A) and hexon DNA content (FIG. 7B) using adenoviral vectors containing fiber AB loop knob and/or penton, PD1 mutations. The following adenoviral vectors were used in these studies: Av1nBg, Av1nBgFKO1, referred to as FKO1, Av1nBgPD1, referred to as PD1, Av1nBgFKO1PD1, referred to as FKO1PD1, Av1nBgKO12, referred to as KO12, and Av1nBgKO12PD1 that is referred to as KO12PD1.

FIG. 8 is a plasmid map for pFBshuttle(EcoRI).

FIG. 9 is a plasmid map for pSQ1HSP.

FIG. 10 is a plasmid map for pSQ1HSPKO1.

FIG. 11 is a plasmid map for pSQ1HSPPD1.

FIG. 12 is a plasmid map for pSQ1HSPKO1PD 1.

FIGS. 13A-13C show the transduction efficiency of A549 and HeLa cells using adenoviral vectors containing fiber shaft, knob and/or penton mutations. FIG. 13A shows the dose response for the transduction efficiency of A549 cells. FIG. 13B shows the transduction efficiency of HeLa cells at 2000 ppc. FIG. 13C shows the competition analysis of adenoviral vectors containing fiber shaft mutations.

FIGS. 14A-14B shows the influence of fiber shaft mutations on in vivo adenoviral-mediated liver gene expression (FIG. 14A) and hexon DNA content (FIG. 14B).

FIGS. 15A-15B are plasmid maps of pSQ1HSPRGD (FIG. 15A) and pSQ1HSPKO1RGD (FIG. 15B).

FIG. 16 shows that insertion of a RGD targeting ligand can restore transduction of the vectors containing the HSP binding shaft S* mutation.

FIGS. 17A-17B are plasmid maps of pSQ1AD35 Fiber (FIG. 17A) and pSQ1Ad35FcRGD (FIG. 17B).

FIGS. 18A-18B are maps of plasmids encoding 35F chimeric fibers. FIG. 18A is a plasmid map of pSQ135T5H, and FIG. 18B is a plasmid map of pSQ15T35H.

FIG. 19 shows the results of an in vitro analysis of Ad5 vectors containing Ad35 fibers and derivatives thereof.

FIG. 20 shows the results of an in vivo analysis of Ad5 vectors containing Ad35 fibers and derivatives thereof.

FIGS. 21A-21B are plasmid maps of pSQ1Ad41sF (FIG. 21A) and pSQ1Ad41sFRGD (FIG. 21B).

FIG. 22 shows the results of an in vivo analysis of Ad5 vectors containing Ad41 short fiber.

FIG. 23 shows the in vitro analysis of Ad5 based vectors containing the Ad41 short fiber which has been re-engineered to contain a cRGD ligand in the HI loop.

FIG. 24 shows enhanced transduction of AE1-2a cells with the Av3nBgFKO1 detargeted adenoviral vector using hexadimethrine bromide (HB), protamine sulfate (PS) and poly-lysine-RGD (K14) or the anti-penton-TNFα bifunctional protein (αpen-TNF).

FIG. 25 shows ablation of HSP interaction decreases adenoviral-mediated gene transfer to other organs

FIG. 26 shows in vivo liver transduction with adenoviral vectors which encode for B-galactosidase and contain various mutations to the fiber and/or penton proteins. Results are plotted as percent transduction as compared to wild type. Two different methods for determining the level of transduction are shown for each vector.

FIG. 27 shows the adenoviral vector biodistribution to the liver and tumor for the vectors containing the S*, KO1S*, and 41sF fibers.

### DETAILED DESCRIPTION

A. Definitions

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information is known and can be readily accessed, such as by searching the internet and/or appropriate databases. Reference thereto evidences the availability and public dissemination of such information.

As used herein, the term “adenovirus” or “adenoviral particle” is used to include any and all viruses that can be categorized as an adenovirus, including any adenovirus that infects a human or an animal, including all groups, subgroups, and serotypes. Depending upon the context reference to “adenovirus” can include adenoviral vectors. There are at least 51 serotypes of Adenovirus that classified into several subgroups. For example, subgroup A includes adenovirus serotypes 12, 18, and 31. Subgroup C includes adenovirus serotypes 1, 2, 5, and 6. Subgroup D includes adenovirus serotype 8, 9, 10, 13, 15, 17, 19, 20, 22-30, 32, 33, 36-39, and 42-49. Subgroup E includes adenovirus serotype 4. Subgroup F includes adenovirus serotypes 40 and 41. These latter two serotypes have a long and a short fiber protein. Thus, as used herein an adenovirus or adenovirus particle is a packaged vector or genome.

As used herein, “virus,” “viral particle,” “vector particle,” “viral vector particle,” and “virion” are used interchangeably to refer to infectious viral particles that are formed when, such as when a vector containing all or a part of a viral genome, is transduced into an appropriate cell or cell line for the generation of such particles. The resulting viral particles have a variety of uses, including, but not limited to, transferring nucleic acids into cells either in vitro or in vivo. For purposes herein, the viruses are adenoviruses, including recombinant adenoviruses formed when an adenovirus vector, such as any provided herein, is encapsulated in an adenovirus capsid. Thus, a viral particle is a packaged viral genome. An adenovirus viral particle is the minimal structural or functional unit of a virus. A virus can refer to a single particle, a stock of particles or a viral genome. The adenovirus (Ad) particle is relatively complex and may be resolved into various substructures.

Included among adenoviruses and adenoviral particles are any and all viruses that can be categorized as an adenovirus, including any adenovirus that infects a human or an animal, including all groups, subgroups, and serotypes. Thus, as used herein, “adenovirus” and “adenovirus particle” refer to the virus itself and derivatives thereof and cover all serotypes and subtypes and naturally occurring and recombinant forms, except where indicated otherwise. Included are adenoviruses that infect human cells. Adenoviruses can be wildtype or can be modified in various ways known in the art or as disclosed herein. Such modifications include, but are not limited to, modifications to the adenovirus genome that is packaged in the particle in order to make an infectious virus. Exemplary modifications include deletions known in the art, such as deletions in one or more of the E1a, E1b, E2a, E2b, E3, or E4 coding regions. Other exemplary modifications include deletions of all of the coding regions of the adenoviral genome. Such adenoviruses are known as “gutless” adenoviruses. The terms also include replication-conditional adenoviruses, which are viruses that preferentially replicate in certain types of cells or tissues but to a lesser degree or not at all in other types. For example, among the adenoviral particles provided herein, are adenoviral particles that replicate in abnormally proliferating tissue, such as solid tumors and other neoplasms. These include the viruses disclosed in U.S. Pat. No. 5,998,205 and U.S. Pat. No. 5,801,029. Such viruses are sometimes referred to as “cytolytic” or “cytopathic” viruses (or vectors), and, if they have such an effect on neoplastic cells, are referred to as “oncolytic” viruses (or vectors).

As used herein, the terms “vector,” “polynucleotide vector,” “polynucleotide vector construct,” “nucleic acid vector construct,” and “vector construct” are used interchangeably herein to mean any nucleic acid construct that can be used for gene transfer, as understood by those skilled in the art.

As used herein, the term “viral vector” is used according to its art-recognized meaning. It refers to a nucleic acid vector construct that includes at least one element of viral origin and can be packaged into a viral vector particle. The viral vector particles can be used for the purpose of transferring DNA, RNA or other nucleic acids into cells either in vitro or in vivo. Viral vectors include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, Sindbis vectors, semliki forest virus vectors, phage vectors, adenoviral vectors, and adeno-associated viral (AAV) vectors. Suitable viral vectors are described, for example, in U.S. Pat. Nos. 6,057,155, 5,543,328 and 5,756,086. The vectors provided herein are adenoviral vectors.

As used herein, “adenovirus vector” and “adenoviral vector” are used interchangeably and are well understood in the art to mean a polynucleotide containing all or a portion of an adenovirus genome. An adenoviral vector, refers to nucleic encoding a complete genome or a modified genome or one that can be used to introduce heterologous nucleic acid when transferred into a cell, particularly when packaged as a particle. An adenoviral vector can be in any of several forms, including, but not limited to, naked DNA, DNA encapsulated in an adenovirus capsid, DNA packaged in another viral or viral-like form (such as herpes simplex, and AAV), DNA encapsulated in liposomes, DNA complexed with polylysine, complexed with synthetic polycationic molecules, conjugated with transferrin, complexed with compounds such as PEG to immunologically “mask” the molecule and/or increase half-life, or conjugated to a non-viral protein.

As used herein, oncolytic adenoviruses refer to adenoviruses that replicate selectively in tumor cells As used herein, a variety of vectors with different requirements and purposes are described. For example, one vector is used to deliver particular nucleic acid molecules into a packaging cell line for stable integration into a chromosome. These types of vectors also are referred to as complementing plasmids. A further type of vector carries or delivers nucleic acid molecules in or into a cell line (e.g., a packaging cell line) for the purpose of propagating viral vectors; hence, these vectors also can be referred to herein as delivery plasmids. A third “type” of vector is the vector that is in the form of a virus particle encapsulating a viral nucleic acid and that is comprised of the capsid modified as provided herein. Such vectors also can contain heterologous nucleic acid molecules encoding particular polypeptides, such as therapeutic polypeptides or regulatory proteins or regulatory sequences to specific cells or cell types in a subject in need of treatment.

As used herein, the term “motif” is used to refer to any set of amino acids forming part of a primary sequence of a protein, either contiguous or capable of being aligned to certain positions that are invariant or conserved, that is associated with a particular function. The motif can occur, not only by virture of the primary sequence, but also as a consequence of three-dimensional folding. For example, the motif GXGXXG is associated with nucleotide-binding sites. In this fiber is a trimer, hence the trimeric structure can contribute formation of a motif. Alternatively, a motif can be considered as a domain of a protein, where domain is a region of a protein molecule delimited on the basis of function without knowledge of and relation to the molecular substructure, as, e.g., the part of a protein molecule that binds to a receptor. As shown herein, the motif KKTK constitutes a consensus sequence for fiber shaft interaction with HSP.

As used herein, the term “bind” or “binding” is used to refer to the binding between a ligand and its receptor, such as the binding of an Ad5 shaft motif with HSP (Heparin Sulfate Proteoglycans), with a K_{d} in the range of 10−2 to 10−15 mole/I, generally, 10⁻⁶ to 10⁻¹⁵, 10⁻⁷ to 10⁻¹⁵ and typically 10⁻⁸ to 10⁻¹⁵ (and/or a Kₐ of 10⁻⁵-10⁻¹², 10⁷-10¹², 10⁸-10¹² I/mole).

As used herein, specific binding or selective binding means that a the binding of a particular ligand and one receptor interaction (kₐ or K_{eq}) is at least 2-fold, generally, 5, 10, 50, 100 or more-fold, greater than for another receptor. A statement that a particular viral vector is targeted to a cell or tissue means that its affinity for such cell or tissue in a host or in vitro is at least about 2-fold, generally, 5, 10, 50, 100 or more-fold, greater than for other cells and tissues in the host or under the in vitro conditions.

As used herein, the term “ablate” or “ablated” is used to refer to an adenovirus, adenoviral vector or adenoviral particle, in which the ability to bind to a particular cellular receptor is reduced or eliminated, generally substantially eliminated (i.e., reduced more than 10-fold, 100-fold or more) when compared to a coresponding wild-type adenovirus. An ablated adenovirus, adenoviral vector or adenoviral particle also is said to be detargeted, i.e., the modified adenovirus, adenoviral vector or adenoviral particle does not possess the native tropism of the wild-type adenovirus. The reduction or elimination of the ability of the mutated adenovirus fiber protein and/or mutated adenovirus penton protein to bind a cellular receptor as compared to the corresponding wild-type fiber protein and/or wild-type penton protein can be measured or assessed by comparing the transduction efficiency (gene transfer and expression of a marker gene) of an adenovirus particle containing the mutated fiber protein and/or mutated penton protein compared to an adenovirus particle containing the wild-type fiber protein and/or wild-type penton protein for cells having the cellular receptor.

As used herein, tropism with reference to an adenovirus refers to the selective infectivity or binding that is conferred on the particle by a capsid protein, such as the fiber protein and/or penton.

As used herein, “penton or penton complex” is used herein to designate a complex of penton base and fiber. The term “penton” can also be used to indicate penton base, as well as penton complex. The meaning of the term “penton” alone should be clear from the context within which it is used.

As used herein, the term “substantially eliminated” refers to a transduction efficiency less than about 11% of the efficiency of the wild-type fiber containing virus on HeLa cells. The transduction efficiency on Hela cells can be measured (see, e.g., Example 1 of U.S. patent application Ser. No. 09/870,203 filed on May 30, 2001, and published as U.S. Published application No. 20020137213, and of International Patent Application No. PCT/EP01/06286 filed Jun. 1, 2001). Briefly, HeLa cells are infected with the adenoviral vectors containing mutated fiber proteins to evaluate the effects of fiber amino acid mutations on CAR interaction and subsequent gene expression. Monolayers of HeLa cells in 12 well dishes are infected with, for example, 1000 particles per cell for 2 hours at 37° C. in a total volume of, for example, 0.35 ml of the DMEM containing 2% FBS. The infection medium is then aspirated from the monolayers and 1 ml of complete DMEM containing 10% FBS was added per well. The cells are incubated for an sufficient time, generally about 24 hours, to allow for β-galactosidase expression, which is measured by a chemiluminescence reporter assay and by histochemical staining with a chromogenic substrate. The relative levels of β-galactosidase activity are determined using as suitable system, such as the Galacto-Light chemiluminescence reporter assay system (Tropix, Bedford, Mass.) Cell monolayers are washed with PBS and processed according to the manufacturer's protocol. The cell homogenate is transferred to a microfuge tube and centrifuged to remove cellular debris. Total protein concentration is determined, such as by using the bicinchoninic acid (BCA) protein assay (Pierce, Inc., Rockford, Ill.) with bovine serum albumin as the assay standard. An aliquot of each sample is then incubated with the Tropix β-galactosidase substrate for 45 minutes in a 96 well plate. A luminometer is used determine the relative light units (RLU) emitted per sample and then normalized for the amount of total protein in each sample (RLU/ug total protein). For the histochemical staining procedure, the cell monolayers are fixed with 0.5% glutaraldehyde in PBS, and then were incubated with a mixture of 1 mg of 5-bromo-4-chloro-3-indolyl-, β-D-galactoside (X-gal) per ml, 5 mM potassium ferrocyanide, 5 mM potassium ferricyanide and 2 mM MgCl₂ in 0.5 ml of PBS. The monolayers are washed with PBS and the blue cells are visualized by light microscopy, such as with a Zeiss IDO3 microscope. Generally, the efficiency is less than about 9%, and typically is less than about 8%.

As used herein, the phrase “reduce” or “reduction” refers to a change in the efficiency of transduction by the adenovirus containing the mutated fiber as compared to the adenovirus containing the wild-type fiber to a level of about 75% or less of the wild-type on HeLa cells. Generally, the change in efficiency is to a level of about 65% or less than wild-type. Typically it is about 55% or less. This system is able to rapidly analyze modified fiber proteins and/or modified penton proteins for desired tropism in the context of the viral particle.

As used herein, the term “mutate” or “mutation” or similar terms refers to the deletion, insertion or change of at least one amino acid in the part of the fiber shaft region interacting with HSP. The amino acid can be changed by substitution or by modification in a way that derivatizes the amino acid. Thus, for example a BBXB motif or BBBXXB motif, where B is a basic amino acid, in an adenovirus is mutated to ablate the viral interaction with HSP.

As used herein, the term “polynucleotide” means a nucleic acid molecule, such as DNA or RNA, that encodes a polynucleotide. The molecule can include regulatory sequences, and is generally DNA. Such polynucleotides are prepared or obtained by techniques known by those skilled in the art in combination with the teachings contained therein.

As used herein, adenoviral genome is intended to include any adenoviral vector or any nucleic acid sequence comprising a modified fiber protein. All adenovirus serotypes are contemplated for use in the vectors and methods herein.

As used herein, the term “viral vector” is used according to its art-recognized meaning. It refers to a nucleic acid vector construct that includes at least one element of viral origin and can be packaged into a viral vector particle. The viral vector particles can be used, for example, for transferring DNA into cells either in vitro or in vivo.

As used herein, a packaging cell line is a cell line that is able to package adenoviral genomes or modified genomes to produce viral particles. It can provide a missing gene product or its equivalent. Thus, packaging cells can provide complementing functions for the genes deleted in an adenoviral genome (e.g., the nucleic acids encoding modified fiber proteins) and are able to package the adenoviral genomes into the adenovirus particle. The production of such particles require that the genome be replicated and that those proteins necessary for assembling an infectious virus are produced. The particles also can require certain proteins necessary for the maturation of the viral particle. Such proteins can be provided by the vector or by the packaging cell.

As used herein, detargeted adenoviral particles have ablated (reduced or eliminated) interaction with receptors with which native particles. Fully detargeted particles have two or more specificities altered. It is understood that in vivo no particles are fully ablated such that they do not interact with any cells. Degareted and fully degarated have reduced, typically substantiall reduced, or eliminated interaction with native receptors. For purposes herein, detargeted particles have reduced (2-fold, 5-fold, 10-fold, 100-fold or more) binding or virtually no binding to HSP receptors; fully degareted vectors include further capsid modifications to eliminate interactions with other receptors, such as CAR and integrins or other receptors. The particles still bind to cells, but the types of cells and interactions are reduced.

As used herein, pseudotyping describes the production of adenoviral vectors having modified capsid protein or capsid proteins from a different serotype than the serotype of the vector itself. One example, is the production of an adenovirus 5 vector particle containing an Ad37 or Ad35 fiber protein. This can be accomplished by producing the adenoviral vector in packaging cell lines expressing different fiber proteins. As provided herein, detargeting of an adenovirus 5 particle or other serotype group C adenovirus or other adenovirus that binds to HSP to reduce or eliminate binding to HSPs can be effected by replacing all or a portion that includes the shaft or at least the HSP consensus binding sequence of the Ad5 fiber with an adenovirus fiber or portion thereof that does not bind to HSP. Adenoviruses having fiber shafts that do not interact with HSP include (a) adenoviruses of subgroup B, e.g., Ad3, Ad35, Ad7, Ad11, Ad16, Ad21, Ad34 which do not have interaction with HSP, (b) adenoviruses of subgroup F, e.g., Ad40 and Ad41, specifically the short fiber, and (c) adenoviruses of subgroup D, e.g., Ad46.

As used herein, receptor refers to a biologically active molecule that specifically or selectively binds to (or with) other molecules. The term “receptor protein” can be used to more specifically indicate the proteinaceous nature of a specific receptor.

As used herein, the term “cyclic RGD” (or cRGD) refers to any amino acid that binds to αᵥ integrins on the surface of cells and contains the sequence RGD (Arg-Gly-Asp).

As used herein, the term “heterologous polynucleotide” means a polynucleotide derived from a biological source other than an adenovirus or from an adenovirus of a different strain or can be a polynucleotide that is in a different locus from wild-type virus. The heterologous polynucleotide can encode a polypeptide, such as a toxin or a therapeutic protein. The heterologous polynucleotide can contain regulatory regions, such as a promoter regions, such as a promoter active in specific cells or tissue, for example, tumor tissue as found in oncolytic adenoviruses. Alternatively, the heterologous polynucleotide can encode a polypeptide and further contain a promoter region operably linked to the coding region.

As used herein, reference to an amino acid in an adenovirus protein or to a nucleotide in an adenovirus genome is with reference to Ad5, unless specified otherwise. Corresponding amino acids and nucleotides in other adenovirus strains and modified strains and in vectors can be identified by those of skill in the art. Thus recitation of a mutation is intended to encompass all adenovirus strains that process a corresponding locus.

As used herein, the KO mutations refer to mutations in fiber that knock out binding to CAR. For example, a KO1 mutation refers to a mutation in the Ad5 fiber and corresponding mutations in other fiber proteins. In Ad5, this mutation results in a substitution of fiber amino acids 408 and 409, changing them from serine and proline to glutamic acid and alanine, respectively. As used herein, a KO12 mutation refers to a mutation in the Ad5 fiber and corresponding mutations in other fiber proteins. In Ad5, this mutation is a four amino acid substitution as follows: R512S, A515G, E516G, and K517G. Other KO mutations can be identified empirically or are known to those of skill in the art.

As used herein, PD mutations refer to mutations in the penton gene that ablate binding by the encoded to αᵥ integrin by replacing the RGD tripeptide. The PD1 mutation exemplified herein results in a substitution of amino acids 337 through 344 of the Ad5 penton protein, HAIRGDTF (SEQ ID No. 9), with amino acids SRGYPYDVPDYAGTS (SEQ ID No. 10), thereby replacing the RGD tripeptide.

As used herein, treatment means any manner in which the symptoms of a condition, disorder or disease are ameliorated or otherwise beneficially altered.

As used herein, a therapeutically effective product is a product that is encoded by heterologous DNA that, upon introduction of the DNA into a host, a product is expressed that effectively ameliorates or eliminates the symptoms, manifestations of an inherited or acquired disease or that cures said disease.

As used herein, a subject is an animal, such as a mammal, typically a human, including patients.

As used herein, genetic therapy involves the transfer of heterologous DNA to the certain cells, target cells, of a mammal, particularly a human, with a disorder or conditions for which such therapy is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product, it may encode a product, such as a peptide or RNA that in some manner mediates, directly or indirectly, expression of a therapeutic product. Genetic therapy may also be used to deliver nucleic acid encoding a gene product to replace a defective gene or supplement a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof.

As used herein, a therapeutic nuucleic acid is a nucleic acid that endes a therapeutic product. The product can be nucleic acid, such as a regulatory sequence or gene, or can encode a protein that has a therapeutic activity or effect. For example, therapeutic nucleic acid can be a ribozyme, antisense, double-stranded RNA, a nucleic acid encoding a protein and others.

As used herein, “homologous” means about greater than 25% nucleic acid sequence identity, such as 25% 40%, 60%, 70%, 80%, 90% or 95%. If necessary the percentage homology will be specified. The terms “homology” and “identity” are often used interchangeably. In general, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule.

Whether any two nucleic acid molecules have nucleotide sequences that are at least, for example, 80%, 85%, 90%, 95%, 96%, 97%, 98% or 99% “identical” can be determined using known computer algorithms such as the “FAST A” program, using for example, the default parameters as in Pearson et al. (1988) Proc. Natl. Acad. Sci. USA 85:2444 (other programs include the GCG program, package (Devereux, J., et al., Nucleic Acids Research 12(1):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids) which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps. Therefore, as used herein, the term “identity” represents a comparison between a test and a reference polypeptide or polynucleotide.

As used herein, the term “at least 90% identical to” refers to percent identities from 90 to 99.99 relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, or deletions. At the level of homologies or identities above about 85-90%, the result should be independent of the program and gap parameters set; such high levels of identity can be assessed readily, often without relying on software.

As used herein: stringency of hybridization in determining percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

Those of skill in this art know that the washing step selects for stable hybrids and also know the ingredients of SSPE (see, e.g., Sambrook, E. F. Fritsch, T. Maniatis, in: Molecular Cloning, A Laboratory Manual, Cold Spring Harbor Laboratory Press (1989), vol. 3, p. B.13, see, also, numerous catalogs that describe commonly used laboratory solutions). SSPE is pH 7.4 phosphate- buffered 0.18 M NaCl. Further, those of skill in the art recognize that the stability of hybrids is determined by Tₘ, which is a function of the sodium ion concentration and temperature (Tₘ=81.5° C.-16.6(log₁₀[Na⁺])+0.41(%G+C)−600/l)), so that the only parameters in the wash conditions critical to hybrid stability are sodium ion concentration in the SSPE (or SSC) and temperature.

It is understood that equivalent stringencies can be achieved using alternative buffers, salts and temperatures. By way of example and not limitation, procedures using conditions of low stringency are as follows (see also Shilo and Weinberg, Proc. Natl. Acad. Sci. USA 78:6789-6792 (1981)): Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA (10×SSC is 1.5 M sodium chloride, and 0.15 M sodium citrate, adjusted to a pH of 7).

Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and reexposed to film. Other conditions of low stringency which can be used are well known in the art (e.g., as employed for cross-species hybridizations).

By way of example and not way of limitation, procedures using conditions of moderate stringency include, for example, but are not limited to, procedures using such conditions of moderate stringency are as follows: Filters containing DNA are pretreated for 6 hours at 55° C. in a solution containing 6×SSC, 5×Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography. Other conditions of moderate stringency which can be used are well-known in the art. Washing of filters is done at 37° C. for 1 hour in a solution containing 2×SSC, 0.1% SDS.

By way of example and not way of limitation, procedures using conditions of high stringency are as follows: Prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1X SSC at 50° C. for 45 minutes before autoradiography. Other conditions of high stringency which can be used are well known in the art.

The term substantially identical or substantially homologous or similar varies with the context as understood by those skilled in the relevant art and generally means at least 60% or 70%, preferably means at least 80%, 85% or more preferably at least 90%, and most preferably at least 95% identity.

As used herein, substantially identical to a product means sufficiently similar so that the property of interest is sufficiently unchanged so that the substantially identical product can be used in place of the product.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

The methods and and preparation of products provided herein, unless otherwise indicated, employ conventional techniques of chemistry, molecular biology, microbiology, recombinant DNA, genetics, immunology, cell biology, cell culture and transgenic biology, which are within the skill of the art (see, e.g., Maniatis et al. (1982) Molecular Cloning: A Laboratory Manual, Cold Spring Harbor Laboratory Press, Cold Spring Harbor, N.Y.); Sambrook et al. (1989) Molecular Cloning: A Laboratory Manual, Cold Spring Harbor Laboratory Press, Cold Spring Harbor, N.Y.; Ausubel et al. (1992) Current Protocols in Molecular Biology, Wiley and Sons, New York; Glover (1985) DNA Cloning I and II, Oxford Press; Anand (1992) Techniques for the Analysis of Complex Genomes (Academic Press); Guthrie and Fink (1991) Guide to Yeast Genetics and Molecular Biology, Academic Press; Harlow and Lane (1988) Antibodies: A Laboratory Manual, Cold Spring Harbor Laboratory, Cold Spring Harobor, N.Y.; Jakoby and Pastan, eds. (1979) Cell Culture. Methods in Enzymology 58, Academic Press, Inc., Harcourt Brace Jaovanovich, N.Y.; Nucleic Acid Hybridization (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal (1984), A Practical Guide To Molecular Cloning; Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Hogan et al. (1986) Manipulating the Mouse Embryo, Cold Spring Harbor Laboratory Press, Cold Spring Harbor, N.Y.

B. Capsid Modifications

Provided herein are modifications of the viral capsid that ablate the interaction of an adenovirus with its natural receptors. In particular, fiber modifications that result in ablation of the interaction of an adenvirus with HSP are provided. These fiber modifications can be combined with other capsid protein modifications, such as other fiber modifications and/or penton and/or hexon modifications, to fully ablate viral interactions with natural receptors, when expressed on a viral particle. The modification should not disrupt trimer formation or transport of fiber into the nucleus.

1. Fiber Genes and Proteins

The fiber protein extends from the capsid and mediates viral binding to the cell surface by binding to specific cell receptors (Philipson et al. (1968) J. Virol. 2:1064-1075). The fiber is a trimeric protein that includes an N-terminal tail domain that interacts with the adenovirus penton base, a central shaft domain of varying length, and a C-terminal knob domain that contains the cell receptor binding site (Chroboczek et al. (1995) Curr. Top. Microbiol. Immunol. 199:163-200; Riurok et al. (1990) J.Mol.Biol. 215:589-596; Stevenson et al. (1995) J. Virol. 69:2850-2857). The sequences of the fiber gene from a variety of serotypes including adenovirus serotypes 2 (Ad2), Ad5, Ad3, Ad35, Ad12, Ad40, and Ad41 are known. There are at least 21 different fiber genes in Genbank.

As noted, the fiber protein can be divided into three domains (see, e.g., Green et al. (1983) EMBO J. 2:1357-1365). The conserved N-terminus contains the sequences responsible for association with the penton base as well as a nuclear localization signal. A rod-like shaft of variable length contains repeats of a 15 amino acid beta structure, with the number of repeats ranging from 6 in Ad3 to 22 in Ad5. A conserved stretch of amino acids which includes the sequence TLWT (SEQ ID No. 36) marks the boundary between the repeating units of beta structure in the shaft and the globular head domain. The C-terminal head domain ranges in size from 157 amino acid residues for the short fiber of Ad41 to 193 residues for Ad11 and Ad34. The fiber spike is a homotrimer and it is thought that the C-terminus is responsible for trimerization of the fiber homotrimer and there are 12 spikes per virion which are attached via association with the penton base complex.

2. Modification of HSP Interaction

The adenovirus fiber protein is a major determinant of adenovirus tropism (Gall et al. (1996) J. Virol. 70:2116-2123; Stevenson et al. (1995) J. Virol. 69:2850-2857). Dogma in the field has been that adenoviral entry occurs via binding to CAR and integrins. This is underscored by published data (Einfeld et al. (2001) J. Virology 75:11284-11291). It is shown herein, however, these published entry pathways are not the predominant ones that act in vivo. Moreover, as shown herein, the dominant entry pathway for hepatocytes in vivo involves a mechanism mediated by the fiber shaft, such as Ad5 shaft, through heparin sulfate proteoglycans binding.

It is shown herein that elimination of this binding eliminates entry vis HSP binding, such as in hepatocytes. Adenoviral fiber shaft modifications that ablate viral interaction with HSP are provided. Thus, as provided herein, efficient detargeting of adenovirus in vivo can be achieved with appropriately designed fiber proteins. Suitable modifications, such as described herein, can be made with respect to any adenovirus in which the wild-type interacts with HSP.

As provided herein, the ability of an adenoviral vector to interact with HSP is modified. In particular, the ability to interact is reduced or eliminated. Modifications include insertions, deletions, individual amino acid mutations and other mutations that alter the structure of the fiber shaft such that the HSP binding of the modified fiber protein is ablated when compared to the HSP binding of the wild-type fiber protein.

In a first aspect of this embodiment, an adenoviral fiber protein is modified by mutating one or more of the amino acids that interact with HSP. For example, the HSP binding motif of the modified fiber protein is no longer able to interact with HSP on the cell surface, thus ablating the viral interaction with HSP. For example, the adenoviral fiber is from a subgroup C adenovirus. Binding to HSP can be eliminated or reduced by mutating the fiber shaft in order to modify the ability of the HSP binding motif, which is, for example, KKTK sequence (SEQ ID No. 1) located between amino acid residues 91 to 94 in the Ad 5 fiber, to interact with HSP. The fiber proteins are modified by chemical and biological techniques known to those skilled in the art, such as site directed mutagenesis of nucleic aicd encoding the fiber or other techniques as illustrated herein.

In another aspect of this embodiment, the ability of a fiber to interact with HSP is modified by replacing the wild-type fiber shaft with a fiber shaft, or portion thereof, of an adenovirus that does not interact with HSP to produce chimeric fiber proteins. The portion is sufficient to reduce or eliminate interaction with HSP. Examples of adenoviruses having fiber shafts that do not interact with HSP include (a) adenoviruses of subgroup B, such as, but are not limited to, Ad3, Ad35, Ad7, Ad11, Ad16, Ad21, Ad34, which do not have interaction with HSP, (b) adenoviruses of subgroup F, such as, but are not limited to, Ad40 and Ad41, specifically the short fiber, and (c) adenoviruses of subgroup D, such as but are not limited to, Ad46. In another embodiment, adenoviral fiber shaft modifications that ablate viral interaction with HSP in combination with adenoviral fiber knob modifications that ablate viral interactions with CAR are provided. Suitable adenoviral fiber modifications include the fiber knob modifications are known to those of skill in the art and are exemplified herein (see, also, U.S. patent application Ser. No. 09/870,203, filed on May 30, 2001, and published as U.S. Published application No. 20020137213, in International Patent Application No. PCT/EP01/06286 filed on Jun. 1, 2001). Modifications of the fiber include mutations of at least one amino acid in the CD loop of a wild-type fiber protein of an adenovirus from subgroup C, D, or E, or the long wild-type fiber of an adenovirus from subgroup F, whereby the ability of a fiber protein to bind to CAR is reduced or substantially eliminated. The fiber proteins with ablated CAR interaction are modified by chemical and biological techniques known to those skilled in the art, as illustrated herein and as described in the above patent application.

Alternatively, adenoviral fiber modifications are made by replacing the wild-type fiber knob with a fiber knob of an adenovirus that does not interact with CAR. The fiber protein also will be selected so that it does not interact with HSP. Examples of adenoviruses having fiber knobs that do not interact with CAR include (a) adenoviruses of subgroup B, e.g., Ad3, Ad35, Ad7, Ad11, Ad16, Ad21, Ad34, (b) adenoviruses of subgroup F, e.g., Ad40 and Ad41, specifically the short fiber.

In another embodiment, adenoviral fiber shaft modifications that ablate viral interaction with HSP in combination with penton modifications that ablate viral interactions with αᵥ integrins are provided. Suitable adenoviral penton modifications include the penton modifications, which are well known to those of skill in the art (see, e.g., U.S. Pat. No. 5,731,190; see, also Einfeld et al. (2001) J. Virology 75:11284-11291; and Bai et al. (1993) J. Virology 67:5198-5205).

For example, penton interaction with αᵥ integrins can be ablated (reduced or eliminated) by substitution of the RGD tripeptide motif, required for αᵥ interaction, in penton with a different tripeptide that does not interact with an αᵥ integrin. The penton proteins with ablated αᵥ integrin interactions are modified by chemical and biological techniques known to those skilled in the art (see, e.g., described U.S. Pat. No. 6,731,190 and as illustrated herein). Generally, the adenovirus is a subgroup B or C adenovirus.

Also provided are adenoviral fiber shaft modifications that ablate viral interaction with HSP in combination with adenoviral fiber knob modifications that ablate viral interactions with CAR and with penton modifications that ablate viral interactions with αᵥ integrins. These modifications are described above and prepared using chemical and biological techniques known to those skilled in the art and as illustrated herein. Generally the adenovirus is a subgroup B or subgroup C adenovirus.

Preparation of fibers modified to eliminate or reduce HSP interactions and fibers modified to alter interactions with other receptors and cell surface proteins, such as CAR and/or αᵥ integrin, is also described in the Examples below. The nucleic acid and/or amino acid sequences of exemplary modified fibers, whose construction are described below) are set forth as SEQ ID Nos. 45-72 as follows:

SEQ ID Nos. 45 and 46 set forth the encoding nucleotide sequence and amino acid sequence of the modified fiber designated 5FKO1, where 5F refers to Adenovirus 5 fiber, KO1 is an exemplary mutation of the CAR interaction site described herein;

SEQ ID Nos. 47 and 48 set forth the encoding nucleotide sequence and amino acid sequence of the modified ber designated 5FKO1RGD, which further includes an RGD ligand to demonstrate retargeting;

SEQ ID Nos. 49 and 50 set forth the encoding nucleotide sequence and amino acid sequence of the modified fiber designated 5FKO12, where 5F refers to Adenovirus 5 fiber, KO12 is another exemplary mutation of the CAR interaction site described herein;

SEQ ID Nos. 51 and 52 set forth the encoding nucleotide sequence and amino acid sequence of the modified fiber designated 5F S* nuc, where 5F refers to Adenovirus 5 fiber, S* is an exemplary mutation of the shaft that alters binding to HSP;

SEQ ID Nos. 53 and 54 set forth the encoding nucleotide sequence and amino acid sequence of the modified fiber designated 5F S*RGD nuc, which further includes an RGD ligand;

SEQ ID Nos. 55 and 56 set forth the encoding nucleotide sequence and amino acid sequence of the modified ber designated 5FKO1S*, which contain the KO1 and S* mutations;

SEQ ID Nos. 57 and 58 set forth the encoding nucleotide sequence and amino acid sequence of the modified fiber designated 5FKO1S*RGD, which further includes an RGD ligand;

SEQ ID Nos. 59 and 60 set forth the encoding nucleotide sequence and amino acid sequence of a Ad35 fiber;

SEQ ID Nos. 61 and 62 set forth the encoding nucleotide sequence and amino acid sequence of the modified fiber designated 35FRGD, which is 35F fiber with an RGD ligand;

SEQ ID Nos. 63 and 64 set forth the encoding nucleotide sequence and amino acid sequence of a Ad41 short fiber;

SEQ ID Nos. 65 and 66 set forth the encoding nucleotide sequence and amino acid sequence of the modified fiber designated 41sFRGD, which is 41 F short fiber with an RGD ligand;

SEQ ID Nos. 67 and 68 set forth the encoding nucleotide sequence and amino acid sequence of Ad5 penton;

SEQ ID Nos. 69 and 70 set forth the encoding nucleotide sequence and amino acid sequence of the modified fiber designated 5TS35H, which is a chimeric fiber in which an Ad5 fiber tail and shaft regions (5TS; amino acids 1 to 403) are connected to an Ad35 fiber head region (35H; amino acids 137 to 323) to form the 5TS35H chimera; and

SEQ ID Nos. 71 and 72 set forth the encoding nucleotide sequence and amino acid sequence of the modified fiber designated 35TS5H, which is a chimeric fiber in which an Ad35 fiber tail and shaft regions (35TS; amino acids 1 to 136) are connected to an Ad5 fiber head region (5H; amino acids 404 to 581) to form the 35TS5H chimera.

SEQ ID No. 1 sets forth the nucleotide sequence of Ad fiber; SEQ ID Nos. 2 and 3 also set forth the coding nucleic acid sequencs for fibers with modified fiber knobs for ablated CAR interaction (see, SEQ ID No. 2 for KO1 and SEQ ID No. 3 for KO12); SEQ ID No. 4 also sets for the encoding nucleic acid sequence of a modified penton for ablated aᵥ integrins (SEQ ID No. 4).

The modified fibers are displayed on virus particles by modifying the fiber protein and optionally additional proteins. This can be achieved by preparing adenoviral vectors that express the modified capsid proteins and produce particles with modified fibers, or by packaging adenoviral vectors, particularly those that do not encode one or more capsid proteins in appropriate packaging lines. Hence, as discussed in detail below, adenoviral vectors and viral particles with modified fibers that do no bind to HSP are provided.

C. Nucleic Acids, Adenoviral Vectors and Cells Containing the Nucleic Acids and Cells Containing the Vectors

Also provided are polynucleotides that encode modified capsid proteins and that encode vectors for preparation of adenovirus that express modified capsid proteins provided herein. The sequences of the wild-type adenovirus proteins are well known in the art and are modified as described herein. Nucleic acid molecules, such as cDNA that encode an exemplary modified fiber knob for ablated CAR interaction (see, SEQ ID No. 2 for KO1 and SEQ ID No. 3 for KO12) and for a modified penton for ablated aᵥ integrins (SEQ ID No. 4) are provided. As discussed above, modified capsid proteins with altered tropism for CAR and αᵥ integrins are known and described in the patents, applications and literature cited herein and known to those of skill in the art (see, e.g., U.S. Pat. No. 5,731,190, U.S. application Ser. No. 09/870,203, published as U.S. Published application No. 20020137213; and Bai et al. (1993) J. Virology 67:5198-5208).

Also provided are vectors including the polynucleotides provided herein. Such vectors include partial or complete adenoviral genomes and plasmids. Such vectors are constructed by techniques known to those skilled in the art and as illustrated herein. Also provided are adenoviral vectors modified by replacing whole fiber protein, or portions thereof, with the fiber proteins, or appropriate portions thereof, of an adenovirus that does not interact with HSP. Adenoviruses that do not interact with HSP can be identified by using the methods described herein which detect binding or non-binding of fiber proteins and adenoviruses with HSP. Among the adenoviral vectors provided herein are those of subgroup C, which include Ad2 and Ad5, in which the nucleic acid encoding the fiber shaft or a portion including the HSP-binding portion is replaced with nucleic acid encoding fiber or an appropriate portion thereof from a serotype, such as Ad35.

Adenoviral fiber modifications, thus, can be made in viral particles by replacing the entire fiber protein with the fiber protein of an adenovirus that does not interact with CAR and/or replacing the HSP binding portion with a portion that does not bind. Generally the adenovirus is a subgroup B or subgroup C adenovirus, and also an adenovirus of subgroup D, such as Ad46. Adenoviral vectors of subgroup C, such as Ad2 and Ad5, having a replaced fiber knob are prepared using techniques well known in the art and as illustrated herein.

1. Preparation of Viral Particles

The packaging cells used to produce the viruses provided herein contain the nucleic acid encoding the capsid protein, including the mutated fiber protein provided herein. Such nucleic acid can be transfected into the cell, generally part of as part of plasmid, or it can be infected into the cell with a viral vector. It can be stably incorporated into the genome of the cell, thus providing for a stable cell line. Alternatively, nucleic acid encoding the mutated capsid protein can be removed from the genome, in which case a transient complementing cell is employed.

The adenovirus genome to be packaged is transferred into the complementing cell by techniques known to those skilled in the art. These techniques include transfection or infection with the adenovirus. The nucleic acid encoding the mutated fiber protein can be in this genome instead of in the packaging cell.

In certain cases, when the nucleic acid encoding the mutated fiber is in the genome to be packaged, it can be desirable for the packaging cell to also encode a fiber protein. Such protein can assist in the maturation and packaging of an infectious particle. Such protein can be a wild-type fiber protein or one modified such that it is unable to attach to the penton base protein and is for use, for example, in producer cells where the fiber is included to provide the packaging function and the vector encodes a full-length fiber.

The packaging cells are cultured under conditions that permit the production of the desired viral particle. The viral particles are recovered by standard techniques. An exemplary method for producing adenoviral particles provided herein is as follows. The nucleic acid encoding the mutated fiber protein is made using standard techniques in an adenoviral shuttle plasmid. This plasmid contains the right end of the virus, in particular from the end of the E3 region through the right ITR. This plasmid is co-transfected into competent cells of an E. coli strain, such as the well known E. coli strain BJ5183 (see, e.g., Degryse (1996) Gene 170:45-50) along with a plasmid, which contains the remaining portion of the adenovirus genome, except for the E1 region and sometimes also the E2a region and also contains a corresponding region of homology. Homologous recombination between the two plasmids generates a full-length plasmid encoding the entire adenoviral vector genome.

This full-length adenoviral vector genome plasmid is then transfected into a complementing cell line. The transfection can be performed in the presence of a reagent that directs adenoviral particle entry into producer cells. Such reagents include, but are not limited to, polycations and bifunctional reagents, such as those described herein. A complementing cell is, for example, is a cell of the PER.C6 cell line, which contains the adenoviral E1 gene (PER.C6 is available, for example, from Crucell, The Netherlands; deposited under ECACC accession no. 96022940; see, also Fallaux et al. (1998) Hum. Gene Ther. 9:1909-1907; see, also, U.S. Pat. No. 5,994,128) or an AE1-2a cell (see, Gorziglia et al. (1996) J. Virology 70:4173-4178; and and Von Seggern et al. (1998) J. Gen. Virol. 79:1461-1468)).

AE1-2a cells are derivatives of the A549 lung carcinoma line (ATCC# CCL 185) with chromosomal insertions of the plasmids pGRE5-2.E1 (also referred to as GRE5-E1-SV40-Hygro construct and listed in SEQ ID No. 41) and pMNeoE2a-3.1 (also referred to as MMTV-E2a-SV40-Neo construct and listed in SEQ ID No. 42), which provide complementation of the adenoviral E1 and E2a functions, respectively.

The 633 cell line (see, von Seggern et al. (2000) J. Virology 74:354-362), which stably expresses the adenovirus serotype 5 wild-type fiber protein, and was derived from the AE1-2a cell line, is another an example of complementing cells. When the cell line is 633 cells, the final passage of adenoviral vector is performed on another complementing cell line (e.g., Per.C6), which does not express wild-type Ad5 fiber.

The transfected complementing cells are maintained under standard cell culture conditions. The adenoviral plasmids recombine to form the adenoviral genome that is packaged. The particles are infectious, but replication deficient because their genome is missing at least the E1 genes. When performed in the 633 cells the particles contain wild-type and mutated fiber proteins. They are recovered from the crude viral lysate, amplified, and are purified by standard techniques.

The recovered particles can be used to infect PER.C6 or AE1-2a cells. This permits the recovery of particles whose capsids contain only the desired mutated fiber. This two-step procedure provides high titer batches of the adenoviral particles provided herein. The adenoviral particles can be replication competent or replication incompetent.

In one embodiment, the particles selectively replicate in certain predetermined target tissue but are replication incompetent in other cells and tissues. In a particular embodiment, the adenoviral particles replicate in abnormally proliferating tissue, such as solid tumors and other neoplasms. In replication conditional adenoviruses, a gene essential for replication is placed under control of a heterologous promoter which is cell or tissue specific. For example, the E1 a gene is placed under control of a promoter which is active in a tumor cell to produce an oncolytic adenovirus or oncolytic adenoviral vector. Administration of oncolytic adenoviral vectors to tumor cells kills the tumor cells. Such replication conditional adenoviral particles and vectors can be produced by techniques known to those skilled in the art, such as those disclosed in the above-referenced U.S. Pat. Nos. 5,998,205 and 5,801,029. These particles and vectors can be produced in adenoviral packaging cells as disclosed above. Generally packaging cells are those that have been designed to limit homologous recombination that could lead to wild-type adenoviral particles. Such cells are well known and include the packaging cell known as PER.C6 (see, e.g., U.S. Pat. Nos. 5,994,128 and 6,033,908; deposited under ECACC accession no. 96022940). Since oncolytic vectors are replication competent in certain cell types, they can be amplified in cell lines derived from said cell type without provision of Ad complementary genes.

2. Adenoviral Vectors and Particles

The adenovirus as used herein for production of the adenoviral vectors and particles can be of any serotype. Adenoviral stocks that can be employed as a source of adenovirus or adenoviral coat protein, such as fiber and/or penton base, can be amplified from the adenoviral serotypes 1 through 47, which are currently available from the American Type Culture Collection (ATCC, Rockville, Md.), or from any other serotype of adenovirus available from any other source. For instance, an adenovirus can be of subgroup A (e.g., serotypes 12, 18, 31), subgroup B (e.g., serotypes 3, 7, 11, 14, 16, 21, 34, 35), subgroup C (e.g., serotypes 1, 2, 5, 6), subgroup D (e.g., serotypes 8, 9, 10, 13, 15, 17, 19, 20, 22-30, 32, 33, 36-39, 42-47), subgroup E (serotype 4), subgroup F (serotype 40, 41), or any other adenoviral serotype.

In certain embodiments, the adenovirus is a subgroup B or a subgroup C adenovirus. Subgroup C adenoviruses which are modified in as described herein, include, but are not limited to, Ad2 and Ad5. For Ad5, the mutation is made in the KKTK sequence (SEQ ID No. 1) located between amino acid residues 91 to 94. The fiber proteins can be modified by chemical and biological techniques known to those skilled in the art. These methods include, but are not limited to, site directed mutagenesis and techniques as illustrated herein.

The adenoviral particle generally includes a targeting ligand as described above. The presence of the targeting ligand permits the delivery of a gene to a desired cell type which is different from the cell type that wild-type adenovirus particles infect or the same as that a wild-type particle infects, but allowing the infection in a selective manner, i.e., non-target cell types are not significantly infected.

The adenoviral vectors provided herein can be used to study cell transduction and gene expression in vitro or in various animal models. The latter case includes ex vivo techniques, in which cells are transduced in vitro and then administered to the animal. They also can be used to conduct gene therapy on humans or other animals. Such gene therapy can be ex vivo or in vivo. For in vivo gene therapy, the adenoviral particles in a pharmaceutically-acceptable carrier are delivered to a human in a therapeutically effective amount in order to prevent, treat, or ameliorate a disease or other medical condition in the human through the introduction of a heterologous gene that encodes a therapeutic protein into cells in such human. The adenoviruses are delivered at a dose ranging from approximately 1 particle per kilogram of body weight to approximately 10¹⁴ particles per kilogram of body weight. Generally, they are delivered at a dose of approximately 10⁶ particles per kilogram of body weight to approximately 10¹³ particles per kilogram of body weight, and typically the dose ranges from approximately 10⁸ particles per kilogram of body weight to approximately 10¹² particles per kilogram of body weight.

Any vectors known to those of skill in the art can be employed and used to produce viral particles that include fibers modified to ablate (including reduce) binding to HSP. Some exemplary vectors are as follows.

a. Gutless Vectors

Gutted adenovirus vectors are those from which most or all viral genes have been deleted. They are grown by co-infection of the producing cells with a “helper” virus (such as using an E1-deleted Ad vector), where the packaging cells expresses the E1 gene products. The helper virus trans-complements the missing Ad functions, including production of the viral structural proteins needed for particle assembly. To incorporate the capsid modifications into a gutted adenoviral vector capsid, the changes must be made to the helper virus as described herein. All the necessary Ad proteins including the modified capsid protein are provided by the modified helper virus, and the gutted adenovirus particles are equipped with the particular modified capsid expressed by the host cells. The E1a, Eb, E2a, E2b and E4 are generally required for viral replication and packaging. If these genes are deleted, then the packaging cell must provide these genes or functional equivalents.

A helper adenovirus vector genome and a gutless adenoviral vector genome are delivered to packaging cells. The cells are maintained under standard cell maintenance or growth conditions, whereby the helper vector genome and the packaging cell together provide the complementing proteins for the packaging of the adenoviral vector particle. Such gutless adenoviral vector particles are recovered by standard techniques. The helper vector genome can be delivered in the form of a plasmid or similar construct by standard transfection techniques, or it can be delivered through infection by a viral particle containing the genome. Such viral particle is commonly called a helper virus. Similarly, the gutless adenoviral vector genome can be delivered to the cell by transfection or viral infection.

The helper virus genome can be the modified adenovirus vector genome as disclosed herein. Such genome also can be prepared or designed so that it lacks the genes encoding the adenovirus E1A and E1B proteins. In addition, the genome can further lack the adenovirus genes encoding the adenovirus E3 proteins. Alternatively, the genes encoding such proteins can be present but mutated so that they do not encode functional E1A, E1B and E3 proteins. Furthermore, such vector genome can not encode other functional early proteins, such as E2A, E2B3, and E4 proteins. Alternatively, the genes encoding such other early proteins can be present but mutated so that they do not encode functional proteins.

In producing the gutless vectors, the helper virus genome is also packaged, thereby producing helper virus. In order the minimize the amount of helper virus produced and maximize the amount of gutless vector particles produced, the packaging sequence in the helper virus genome can be deleted or otherwise modified so that packaging of the helper virus genome is prevented or limited. Since the gutless vector genome will have an unmodified packaging sequence, it will be preferentially packaged.

One way to do this is to mutate the packaging sequence by deleting one or more of the nucleotides comprising the sequence or otherwise mutating the sequence to inactivate or hamper the packaging function. One exemplary approach is to engineer the helper genome so that recombinase target sites flank the packaging sequence and to provide a recombinase in the packaging cell. The action of recombinase on such sites results in the removal of the packaging sequence from the helper virus genome. The recombinase can be provided by a nucleotide sequence in the packaging cell that encodes the recombinase. Such sequence can be stably integrated into the genome of the packaging cell. Various kinds of recombinase are known by those skilled in the art, and include, but are not limited to, Cre recombinase, which operates on so-called lox sites, which are engineered on either side of the packaging sequence as discussed above (see, e.g., U.S. Pat. Nos. 5,919, 676, 6,080,569 and 5,919,676; see, also, e.g., Morsy and Caskey, Molecular Medicine Today, January 1999, pgs. 18-24).

An example of a gutless vector is pAdARSVDys (Haecker et aL (1996) Hum Gene Ther. 7:1907-1914)). This plasmid contains a full-length human dystrophin cDNA driven by the RSV promoter and flanked by Ad inverted terminal repeats and packaging signals. 293 cells are infected with a first-generation Ad, which serves as a helper virus, and then transfected with purified pAdARSVDys DNA. The helper Ad genome and the pAdARSVDys DNA are replicated as Ad chromosomes, and packaged into particles using the viral proteins produced by the helper virus. Particles are isolated and the pAdARSVDys-containing particles separated from the helper by virtue of their smaller genome size and therefore different density on CsCl gradients. Other examples of gutless adenoviral vectors are known (see, e.g., Sandig et al. (2000) Proc. Natl. Acad. Sci. U.S.A. 97(3):1002-7).

b. Oncolytic Vectors

Briefly, oncolytic adenoviruses, which are viruses that replicate selectively in tumor cells, are designed to amplify the input virus dose due to viral replication in the tumor, leading to spread of the virus throughout the tumor mass. In situ replication of adenoviruses leads to cell lysis. This in situ replication permits relatively low, non-toxic doses to be highly effective in the selective elimination of tumor cells. One approach to achieving selectivity is to introduce loss-of-function mutations in viral genes that are essential for growth in non-target cells but not in tumor cells. (See, e.g., U.S. Pat. No. 5,801,029.) This strategy is exemplified by the use of Addl1520, which has a deletion in the E1b-55 KD gene. In normal cells, the adenoviral E1b-55 KD protein is needed to bind to p53 to prevent apoptosis. In p53-deficient tumor cells, E1b-55K binding to p53 is unnecessary. Thus, deletion of E1b-55 KD should restrict vector replication to p53-deficient tumor cells.

Another approach is to use tumor-selective promoters to control the expression of early viral genes required for replication (see, e.g., International PCT application Nos. WO 96/17053 and WO 99/25860). Thus, in this approach the adenoviruses selectively replicate and lyse tumor cells if the gene that is essential for replication is under the control of a promoter or other transcriptional regulatory element that is tumor-selective.

For example oncolytic adenoviral vectors that contain a cancer selective regulatory region operatively linked to an adenoviral gene essential for adenoviral replication are known (see, e.g., U.S. Pat. No. 5,998,205). Adenoviral genes essential for replication include, but are not limited to, E1a, E1b, E2a, E2b and E4. For example, an exemplary oncolytic adenoviral vector has a cancer selective regulatory region operatively linked to the E1a gene. In other embodiments, the oncolytic adenoviral vector has a cancer selective regulatory region of the present invention operatively linked to the E1a gene and a second cancer selective regulatory region operatively linked to the E4 gene. The vectors also can include at least one therapeutic transgene, such as, but not limited to, a polynucleotide encoding a cytokine such as GM-CSF that can stimulate a systemic immune response against tumor cells.

Other exemplary oncolytic adenoviral vectors include those in which expression of an adenoviral gene, which is essential for replication, is controlled by E2F-responsive promoters, which are selectively transactivated in cancer cells. Thus, vectors that contains an adenoviral nucleic acid backbone that contains in sequential order: A left ITR, an adenoviral packaging signal, a termination signal sequence, an E2F responsive promoter which is operably linked to a first gene, such as E1a, essential for replication of the recombinant viral vector and a right ITR (see, published International PCT application No. WO02/06786, and U.S. Pat. No. 5,998,205).

In other embodiments, the oncolytic adenoviral vector has a cancer selective regulatory region operatively linked to the E1a gene and a second cancer selective regulatory region operatively linked to the E4 gene. The vectors can also carry at least one therapeutic transgene, such as, but not limited to, a polynucleotide encoding a cytokine such as GM-CSF that can stimulate a systemic immune response against tumor cells.

3. Packaging

The viral particles provided herein can be made by any method known to those of skill in the art. Generally they are prepared by growing the adenovirus vector that contains nucleic acid that encodes the modified fiber protein in a standard adenovirus packaging cells to produce particles that express the modified fibers. Alternatively, the vectors do not encode fibers. Such vectors are packaged in producer cells to produce particles that express the modified fiber proteins.

As discussed, recombinant adenoviral vectors generally have at least a deletion in the first viral early gene region, referred to as E1, which includes the E1a and E1b regions. Deletion of the viral E1 region renders the recombinant adenovirus defective for replication and incapable of producing infectious viral particles in subsequently-infected target cells. Thus, to generate E1-deleted adenovirus genome replication and to produce virus particles requires a system of complementation which provides the missing E1 gene product. E1 complementation is typically provided by a cell line expressing E1, such as the human embryonic kidney packaging cell line, i.e. an epithelial cell line, called 293. Cell line 293 contains the E1 region of adenovirus, which provides E1 gene region products to “support” the growth of E1-deleted virus in the cell line (see, e g., Graham et al., J. Gen. Virol. 36: 59-71, 1977). Additionally, cell lines that may be usable for production of defective adenovirus having a portion of the adenovirus E4 region have been reported (WO 96/22378). Multiply deficient adenoviral vectors and complementing cell lines have also been described (WO 95/34671, U.S. Pat. No. 5,994,106).

For example, copending U.S. application Ser. No. 09/482,682 (also filed as International PCT application No. PCT/EP00/00265, filed Jan. 14, 200, published as International PCT application No. WO/0042208) provides packaging cell lines that support viral vectors with deletions of major portions of the viral genome, without the need for helper viruses and also provides cell lines and helper viruses for use with helper-dependent vectors. The packaging cell line has heterologous DNA stably integrated into the chromosomes of the cellular genome. The heterologous DNA sequence encodes one or more adenovirus regulatory and/or structural polypeptides that complement the genes deleted or mutated in the adenovirus vector genome to be replicated and packaged.

Packaging cell lines express, for example, one or more adenovirus structural proteins, polypeptides, or fragments thereof, such as penton base, hexon, fiber, polypeptide Illa, polypeptide V, polypeptide VI, polypeptide VII, polypeptide VIII, and biologically active fragments thereof. The expression can be constitutive or under the control of a regulatable promoter. These cell lines are particularly designed for expression of recombinant adenoviruses intended for delivery of therapeutic products. For use herein, such packaging cell lines can express the modified capsid proteins, such as the fiber proteins who binding to HSP is reduced or eliminated, and/or the modified penton and hexon proteins.

Particular packaging cell lines complement viral vectors having a deletion or mutation of a DNA sequence encoding an adenovirus structural protein, regulatory polypeptides E1A and E1B, and/or one or more of the following regulatory proteins or polypeptides: E2A, E2B, E3, E4, L4, or fragments thereof.

The packaging cell lines are produced by introducing each DNA molecule into the cells and then into the genome via a separate complementing plasmid or plurality of DNA molecules encoding the complementing proteins can be introduced via a single complementing plasmid. Of interest herein, is a variation in which the complementing plasmid includes DNA encoding adenovirus fiber protein (or a chimeric or modified variant thereof), from Ad virus of subgroup D, such as Ad 37, polypeptide or fragment thereof.

For applications, such as therapeutic applications, the delivery plasmid further can include a nucleotide sequence encoding a heterologous polypeptide. Exemplary delivery plasmids include, but are not limited to, pDV44, pAE1B, 8-gal and pAE1sp1B. In a similar or analogous manner, therapeutic nucleic acids, such as nucleic acids that encode therapeutic genes, can be introduced.

The cell further includes a complementing plasmid encoding a fiber as contemplated herein; the plasmid or portion thereof is integrated into a chromosome(s) of the cellular genome of the cell.

Typically, the packaging cell lines will contain nucleic acid encoding the fiber protein or modified protein stably integrated into a chromosome or chromosomes in the cellular genome. The packaging cell line can be derived from a procaryotic cell line or from a eukaryotic cell line. While various embodiments suggest the use of mammalian cells, and more particularly, epithelial cell lines, a variety of other, non-epithelial cell lines are used in various embodiments. Thus, while various embodiments disclose the use of a cell line selected from among the 293, A549, W162, HeLa, Vero, 211, and 211A cell lines, and any other cell lines suitable for such use are likewise contemplated herein.

D. Addition of a Targeting Ligand

The viral particles that are detargeted as described herein, can be retargeted to selected cells and/or tissues by inclusion of an appropriate targeting ligand in the capsid. The ligand cam be included in any of the capsid proteins, such as fiber, hexon and penton. Loci for inclusion of nucleic acid encoding a is known to those of skill in the art for a a variety of adenovirus serotypes; if necessary appropriate loci and other parameters can be empirically determined.

The ligand can be produced as a fusion by inclusion of the coding sequences in the nucleic acid encoding a capsid protein, or chemically conjugated, such as via ionic, covalent or other interactions, to the capsid or bound to the capsid (e.g., by Ab-ligand fusion, where Ab binds capsid protein; or by disulfide bonding or other crosslinking moieties or chemistries).

Thus, for example, a modified fiber nucleic acid also can include sequences of nucleotides that encode a targeting ligand to produce viral particles that include a targeting ligand in the capsid. Targeting ligand and methods for including such ligands in viral capids are well known. For example, inclusion of targeting ligands in fiber proteins is described in U.S. Pat. Nos. 5,543,328 and 5,756,086 and in U.S. patent application Ser. No. 09/870,203, published as U.S. Published application No. 20020137213, and International Patent Application No. PCT/EP01/06286. For different serotypes and strains of adenoviruses, loci for insertion of targeting ligands can be empirically determined. For different serotypes and strains, such loci can vary.

Because the adenovirus fiber has a trimeric structure, the ligand can be selected or designed to have a trimeric structure so that up to three molecules of the ligand are present for each mature fiber. Such ligands can be incorporated into the fiber protein using methods known in the art (see, e.g., U.S. Pat. No. 5,756,086). Instead of the fiber, the targeting ligand can be included in the penton or hexon proteins. Inclusion of targeting ligands in penton (see for example, in U.S. Pat. Nos. 5,731,190 and 5,965,431) and in hexon (see for example, in U.S. Pat. No. 5,965,541) is known.

In one exemplary embodiment, the ligand is included in a fiber protein, which is a fiber protein mutated as described herein. As shown herein, the targeting ligand can be included, for example, within the Hi loop of the fiber protein. Any ligand that can fit in the HI loop and still provide a functional virus is contemplated herein. Such ligands can be as long as or longer than 80-100 amino acids (see, e.g., Belousova et al. (2002) J. Virol. 76:8621-8631). Such ligands are added by techniques known in the art (see, e.g., published International Patent Application publication No. WO99/39734 and U.S. patent application Ser. No. 09/482,682). Other ligands can be be discovered through techniques known to those skilled in the art. Some non-limiting examples of these techniques include phage display libraries or by screening other types of libraries.

Targeting ligands include any chemical moiety that preferentially directs an adenoviral particle to a desired cell type and/or tissue. The categories of such ligands include, but are not limited to, peptides, polypeptides, single chain antibodies, and multimeric proteins. Specific ligands include the TNF superfamily of ligands which include tumor necrosis factors (or TNF's) such as, for example, TNFα and TNFβ, lymphotoxins (LT), such as LT-α and LT-β, Fas ligand which binds to Fas antigen; CD40 ligand, which binds to the CD40 receptor of B-lymphocytes; CD30 ligand, which binds to the CD30 receptor of neoplastic cells of Hodgkin's lymphoma; CD27 ligand, NGF ligand, and OX-40 ligand; transferrin, which binds to the transferrin receptor located on tumor cells, activated T-cells, and neural tissue cells; ApoB, which binds to the LDL receptor of liver cells; alpha-2-macroglobulin, which binds to the LRP receptor of liver cells; alpha-I acid glycoprotein, which binds to the asialoglycoprotein receptor of liver; mannose-containing peptides, which bind to the mannose receptor of macrophages; sialyl-Lewis-X antigen-containing peptides, which bind to the ELAM-I receptor of activated endothelial cells; CD34 ligand, which binds to the CD34 receptor of hematopoietic progenitor cells; ICAM-I, which binds to the LFA-I (CD11b/CD18) receptor of lymphocytes, or to the Mac-I (CD11a/CD18) receptor of macrophages; M-CSF, which binds to the c-fms receptor of spleen and bone marrow macrophages; circumsporozoite protein, which binds to hepatic Plasmodium falciparum receptor of liver cells; VLA-4, which binds to the VCAM-I receptor of activated endothelial cells; HIV gp120 and Class II MHC antigen, which bind to the CD4 receptor of T -helper cells; the LDL receptor binding region of the apolipoprotein E (ApoE) molecule; colony stimulating factor, or CSF, which binds to the CSF receptor; insulin-like growth factors, such as IGF-I and IGF-II, which bind to the IGF-I and IGF-II receptors, respectively; Interleukins 1 through 14, which bind to the Interleukin 1 through 14 receptors, respectively; the Fv antigen-binding domain of an immunoglobulin; gelatinase (MMP) inhibitor; bombesin, gastrin-releasing peptide; substance P; somatostatin; luteinizing hormone releasing hormone (LHRH); vasoactive peptide (VIP); gastrin; melanocyte stimulating hormone (MSH); cyclic RGD peptide and any other ligand or cell surface protein-binding (or targeting) molecule.

E. Heterologous Polynucleotides and Therapeutic Nucleic Acids

The packaged adenoviral genome also can contain a heterologous polynucleotide that encodes a product of interest, such as a therapeutic protein. Adenoviral genomes containing heterologous polynucleotides are well known (see, e.g., U.S. Pat. Nos. 5,998,205, 6,156,497, 5,935,935, and 5,801,029). These can be used for in vitro and in vivo delivery of the products of heterlogous polynucleoties or the heterologous polynucleotides.

Thus, the adenoviral particles provided herein can be used to engineer a cell to express a protein that it otherwise does not express or does not express in sufficient quantities. This genetic engineering is accomplished by infecting the desired cell with an adenoviral particle whose genome includes a desired heterologous polynucleotide. The heterologous polynucleotide is then expressed in the genetically engineered cells. For use herein the cell is generally a mammalian cell, and is typically a primate cell, including a human cell. The cell can be inside the body of the animal (in vivo) or outside the body (in vitro). Heterologous polynucleotides (also referred to as heterologous nucleic acid sequences) are included in the adenoviral genome within the particle and are added to that genome by techniques known in the art. Any heterologous polynucleotide of interest can be added, such as those disclosed in U.S. Pat. No. 5,998,205, incorporated herein by reference.

Polynucleotides that are, introduced into an Ad genome or vector can be any that encode a protein of interest or that are regulatory sequences. Proteins include, but are not limited to, therapeutic proteins, such as an immunostimulating protein, such as an interleukin, interferon, or colony stimulating factor, such as granulocyte macrophage colony stimulating factor (GM-CSF; see, e.g., 5,908,763F. Generally, such GM-CSF is a primate GM-CSF, including human GM-CSF. Other immuno-stimulatory genes include, but are not limited to, genes that encode cytokines IL1, IL2, IL4, IL5, IFN, IFN, TNF, IL12, IL18, and flt3), proteins that stimulate interactions with immune cells (B7, CD28, MHC class I, MHC class II, TAPs), tumor-associated antigens (immunogenic sequences from MART-1, gp100(pmel-17), tyrosinase, tyrosinase-related protein 1, tyrosinase-related protein 2, melanocyte-stimulating hormone receptor, MAGE1, MAGE2, MAGE3, MAGE12, BAGE, GAGE, NY-ESO-1, -catenin, MUM-1, CDK-4, caspase 8, KIA 0205, HLA-A2R17O1, -fetoprotein, telomerase catalytic protein, G-250, MUC-1, carcinoembryonic protein, p53, Her2/neu, triosephosphate isomerase, CDC-27, LDLR-FUT, telomerase reverse transcriptase, and PSMA), cDNAs of antibodies that block inhibitory signals (CTLA4 blockade), chemokines (MIP1, MIP3, CCR7 ligand, and calreticulin), and other proteins.

Other polynucleotides, including therapeutic nucleic acids, such as therapeutic genes, of interest include, but are not limited to, anti-angiogenic, and suicide genes. Anti-angiogenic genes include, but are not limited to, genes that encode METH-1, METH -2, TrpRS fragments, proliferin-related protein, prolactin fragment, PEDF, vasostatin, various fragments of extracellular matrix proteins and growth factor/cytokine inhibitors. Various fragments of extracellular matrix proteins include, but are not limited to, angiostatin, endostatin, kininostatin, fibrinogen-E fragment, thrombospondin, tumstatin, canstatin, and restin. Growth factor/cytokine inhibitors include, but are not limited to, VEGF/VEGFR antagonist, sFlt-1, sFlk, sNRP1, angiopoietin/tie antagonist, sTie-2, chemokines (IP-10, PF-4, Gro-beta, IFN-gamma (Mig), IFN, FGF/FGFR antagonist (sFGFR), Ephrin/Eph antagonist (sEphB4 and sephrinB2), PDGF, TGF and IGF-1.

A “suicide gene” encodes a protein that can lead to cell death, as with expression of diphtheria toxin A, or the expression of the protein can render cells selectively sensitive to certain drugs, e.g., expression of the Herpes simplex thymidine kinase gene (HSV-TK) renders cells sensitive to antiviral compounds, such as acyclovir, gancyclovir and FIAU (1-(2-deoxy-2-fluoro-.beta.-D-arabinofuranosil)-5-iodouracil). Other suicide genes include, but are not limited to, genes that encode carboxypeptidase G2 (CPG2), carboxylesterase (CA), cytosine deaminase (CD), cytochrome P450 (cyt-450), deoxycytidine kinase (dCK), nitroreductase (NR), purine nucleoside phosphorylase (PNP), thymidine phosphorylase (TP), varicella zoster virus thymidine kinase (VZV-TK), and xanthine-guanine phosphoribosyl transferase (XGPRT). Alternatively, a therapeutic nucleic acid can exert its effect at the level of RNA, for instance, by encoding an antisense message or ribozyme, a protein that affects splicing or 3′ processing (e.g., polyadenylation), or a protein that affects the level of expression of another gene within the cell, e.g. by mediating an altered rate of mRNA accumulation, an alteration of mRNA transport, and/or a change in post-transcriptional regulation. The addition of a therapeutic nucleic acid to a virus results in a virus with an additional antitumor mechanism of action. Thus, a single entity (i.e., the virus carrying a therapeutic transgene) is capable of inducing multiple antitumor mechanisms. Other encoded proteins, include, but are not limited to, herpes simplex virus thymidine kinase (HSV-TK), which is useful as a safety switch (see, U.S. patent application Ser. No. 08/974,391, filed Nov. 19, 1997, which published as PCT Publication No. WO/9925860), Nos, FasL, and sFasR (soluble Fas receptor).

Also contemplated are combinations of two or more transgenes with synergistic, complementary and/or nonoverlapping toxicities and methods of action. The resulting adenovirus can retain the viral oncolytic functions and, for example, additionally are endowed with the ability to induce immune and anti-angiogenic responses and other responses as desired.

Therapeutic polynucleotides and heterologous polynucleotides also include those that exert an effect at the level of RNA or protein. These include include a factor capable of initiating apoptosis, RNA, such as RNAi and other double-stranded RNA, antisense and ribozymes, which among other capabilities can be directed to mRNAs encoding proteins essential for proliferation, such as structural proteins, transcription factors, polymerases, genes encoding cytotoxic proteins, genes that encode an engineered cytoplasmic variant of a nuclease (e.g. RNase A) or protease (e.g. trypsin, papain, proteinase K and carboxypeptidase). Other polynucleotides include a cell or tissue specific promoters, such as those used in oncolytic adenoviruses (see, e.g., U.S. Pat. No. 5,998,205).

The heterologous polynucleotide encoding a polypeptide also can contain a promoter operably linked to the coding region. Generally the promoter is a regulated promoter and transcription factor expression system, such as the published tetracycline-regulated systems, or other regulatable systems (WO 01/30843), to allow regulated expression of the encoded polypeptide. Exemplary of other promoters, are tissue-selective promoters, such as those described in U.S. Pat. No. 5,998,205. An exemplary regulatable promoter system is the Tet-On(and Tet-Off( systems currently available from Clontech (Palo Alto, Calif.). This promoter system allows the regulated expression of the transgene controlled by tetracycline or tetracycline derivatives, such as doxycycline. This system can be used to control the expression of the encoded polypeptide in the viral particles and nucleic acids provided herein. Other regulatable promoter systems are known (see, e.g., published U.S. No. 20020168714, entitled “Regulation of Gene Expression Using Single-Chain, Monomeric, Ligand Dependent Polypeptide Switches,” which describes gene switches that contain ligand binding domains and transcriptional regulating domains, such as those from hormone receptors). Other suitable promoters that can be employed include, but are not limited to, adenoviral promoters, such as the adenoviral major late promoter and/or the E3 promoter; or heterologous promoters, such as the cytomegalovirus (CMV) promoter; the Rous Sarcoma Virus (RSV) promoter; inducible promoters, such as the MMT promoter, the metallothionein promoter; heat shock promoters; the albumin promoter; and the ApoAI promoter.

Therapeutic transgenes can be included in the viral constructs and resulting particles. Among these are those that result in an “armed” virus. For example, rather than delete E3 region as in some embodiments described herein, all or a part of the E3 region can be preserved or re-inserted in an oncolytic adenoviral vector (discussed above). The presence of all or a part of the E3 region can decrease the immunogenicity of the adenoviral vector. It also increases cytopathic effect in tumor cells and decreases toxicity to normal cells. Typically such vector expresses more than half of the E3 proteins.

Adenoviruses for therapy, including those for human therapy, are known. Such known viruses can be modified as provided herein to reduce or eliminate interaction with HSPs and optionally additional receptors. The adenoviral vectors that are used to produce the viral particles can include other modifications. Modifications include modifications to the adenovirus genome that is packaged in the particle in order to make an adenoviral vector. As discussed above, adenovirus vectors and particles with a variety of modifications are available. Modifications to adenvoiral vectors include deletions known in the art, such as deletions in one or more of the E1, E2a, E2b, E3, or E4 coding regions. These adenoviruses are sometimes referred to as early generation adenoviruses include those with deletions of all of the coding regions of the adenoviral genome (“gutless” adenoviruses, discussed above) and also include replication-conditional adenoviruses, which are viruses that replicate in certain types of cells or tissues but not in other types as a result of placing adenoviral genes essential for replication under control of a heterologous promoter (discussed above; see, also U.S. Pat. No. 5,998,205, U.S. Pat. No. 5,801,029; U.S. Pat. No. application No. 60/348,670 and corresponding published International PCT application No. WO02/06786). These include the cytolytic, cytopathic viruses (or vectors), including the oncolytic viruses discussed above.

Alternatively, as discussed above, the vector can include a mutation or deletion in the E1b gene. Typically such mutation or deletion in the E1b gene is such that the E1b-19 kD protein becomes non-functional. This modification of the E1b region can be combined with vectors where all or a part of the E3 region is present.

The oncolytic adenoviral vector can further include at least one heterologous coding sequence, such as one that encodes a therapeutic product. The heterologous coding sequence, such as therapeutic gene, is generally, although not necessarily, in the form of cDNA, and can be inserted at any locus that does not adversely affect the infectivity or replication of the vector. For example, it can be inserted in an E3 region in place of at least one of the polynucleotide sequences that encode an E3 protein, such as, for example, the 19 kD or 14.7 kD E3 gene.

F. Propagation and Scale-Up

Since doubly ablated adenoviral vectors containing mutations in the fiber and/or penton capsid proteins result in inefficient cell binding and entry via the CAR/αv integrin entry pathway, scaled up technologies improve the growth and propagation of such vectors to produce high titers of the adenoviral vectors for clinical use. Thus, also provided is a method for scaling up the production of detargeted adenoviral vectors. The detargeted adenoviral vectors comprise an adenoviral vector modified to ablate the interaction of said vector with at least one host cell receptor compared with a wild-type adenoviral vector. The detargeted adenoviral vectors can comprise an adenoviral vector modified to ablate the interaction of said vector with one, two, three or more host cell receptors. Thus, the method is suitable for producing the detargeted adenoviral vectors disclosed herein.

As noted, growth and propagation of doubly and fully ablated adenoviral vectors is enhanced by new scale up technologies. Doubly ablated vectors contain mutations in the fiber and penton capsid proteins that result in inefficient cell binding and entry via the normal cellular entry pathway using CAR and integrins. These vectors are fully detargeted in vitro and, thus, alternative cellular entry strategies allow for the efficient growth and generation of high titer preparations.

Two strategies have been envisioned to scale up vectors that are detargeted via fiber and/or penton modifications. These include: (a) the use of pseudoreceptor cell lines engineered to express a surface receptor that binds a ligand displayed on the vector (see, e.g., International PCT application No. WO 98/54346) and (b) complementing cell lines that are engineered to express native fiber and that can be engineered to express native fiber and penton (see, e.g., International PCT application No. WO 00/42208). Although these systems have shown promise for scaling up ablated adenoviral vectors, there is a need to develop a system for the simple, efficient production of the fully detargeted adenoviral vector for therapeutic uses.

Provided herein is a scale-up method for the propagation of detargeted adenoviral vectors. The method uses polycations and/or bifunctional reagents, which when added to tissue culture medium, bind adenoviral particles and direct their entry into the producer cells.

Reagents (also called medium additives) also can be included in the tissue culture medium containing producer cells to be infected with the detargeted adenoviral vectors. Alternatively the reagents can be pre-mixed with the virus, which mixture is then added to the tissue producer cells. The reagents can be added to tissue culture medium containing producer cells, or producer cells can be added to tissue culture medium containing the reagents. Any suitable producer cell known to the skilled artisan can be used in the present methods. The reagents can be added at the same time that the producer cells are infected with detargeted adenoviral vectors. Generally the reagents are present in the tissue culture medium prior to infection by the detargeted adenoviral vectors. The medium additives are maintained in the tissue culture medium during vector growth, spread and propagation. High titer yields of adenoviral vectors are obtained by this method.

Reagents which are useful in this method are those that are capable of directing adenoviral particle entry into the producer cells. Such reagents include, but are not limited to, polycations and bifunctional reagents. Suitable polycations include, but are not limited to, polytheylenimine; protamine sulfate; poly-L-lysine hydrobromide; poly(dimethyl diallyl ammonium) chloride (Merquat(r)-100, Merquat(r)280, Merquat(r)550); poly-L-arginine hydrochloride; poly-L-histidine; poly(4-vinylpyridine), poly(4-vinylpyridine) hydrochloride; poly(4-vinyl-pyridine)cross-linked, methylchloride quaternary salt; poly(4-vinyl-pyridine-co-styrene); poly(4-vinylpyridinium poly(hydrogen fluoride)); poly(4-vinylpyridinium-P-toluene sulfonate); poly(4-vinylpyridinium-tribromide); poly(4-vinylpyrrolidone-co-2-dimethylamino-ethyl methacrylate); polyvinylpyrrolidone, cross-linked; poly vinylpyrrolidone, poly(melamine-co-formaldehyde); partially methylated; hexadimethrine bromide; poly(Glu, Lys) 1:4 hydrobromide; poly(Lys, Ala) 3:1 hydrobromide; poly(Lys, Ala) 2:1 hydrobromide; poly-L-lysine succinylated; poly(Lys, Ala) 1:1 hydrobromide; and poly(Lys, Trp) 1:4 hydrobromide.

Suitable bifunctional reagents include, but are not limited to, antibodies or peptides that bind to the adenoviral capsid and that also contain a ligand that allows interaction with specific cell surface receptors of the producer cells. Examples of bifunctional reagents include: (a) anti-fiber antibody ligand fusions, (b) anti-fiber-Fab-FGF conjugate, (c) anti-penton-antibody ligand fusions, (d) anti-hexon antibody ligand fusions and (e) polylysine-peptide fusions. The ligand is any ligand that will bind to any cell surface receptor found on the producer cells.

The following examples are included for illustrative purposes only and are not intended to limit the scope of the invention.

### EXAMPLE 1

Construction of Ad5 Vectors Containing the Fiber AB Loop, KO1 and Penton, PD1 Mutations and Derivatives Thereof

Three recombinant adenoviral vectors were prepared that contain the KO1 fiber or PD1 penton base mutations either alone or in combination, these vectors are designated Av3nBgFKO1 Av1nBgPD1, and Av1nBgFKO1PD1. Construction of these vectors is described below and a general description of each vector is set forth in Table 1.

TABLE I
Description Of Detargeted Recombinant Adenoviral
Vectors Used For Scale-up Vector

| Vector | Description |
|---|---|
| Av3nBg | An E1, E2a, E3-deleted adenoviral vector encoding |
| | a nuclear localizing β-galactosidase |
| Av1nBg | An E1 and E3-deleted adenoviral vector encoding |
| | a nuclear localizing β-galactosidase |
| Av3nBgFKO1 | The same as Av3nBg but containing the KO1 |
| | mutation in the fiber gene |
| Av1nBgPD1 | The same as Av1nBg but containing the PD1 |
| | mutation in the penton gene |
| Av1nBgFKO1PD1 | The same as Av1nBg but containing the |
| | fiber KO1 and penton PD1 mutations |

Av1nBg

This is a well-known vector, its sequence is set forth in SEQ ID No. 43.

Av3nBg

This is a well-known vector, its sequence is set forth in SEQ ID No. 44.

Av3nBgFKO1

Genetic incorporation of the KO1 fiber mutation to generate Av3nBgFKO1

The adenoviral vector Av3nBgFKO1 was generated in an E1-, E2a-, E3-deleted backbone based on the adenovirus serotype 5 genome. It contains a RSV promoted nuclear-localizing β-galactosidase gene in place of the E1 region. In addition, the fiber gene carries the KO1 mutation. This mutation results in a substitution of fiber amino acids 408 and 409, changing them from serine and proline to glutamic acid and alanine, respectively.

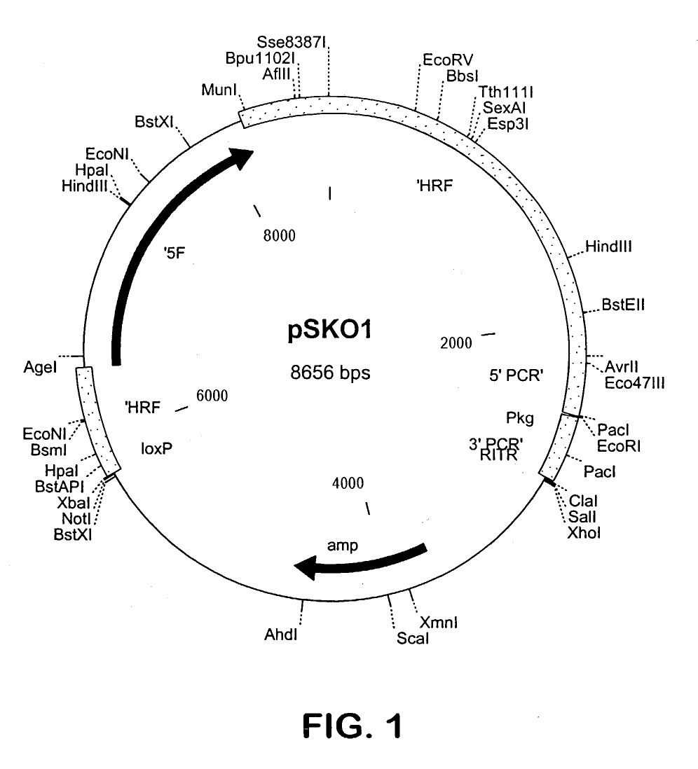

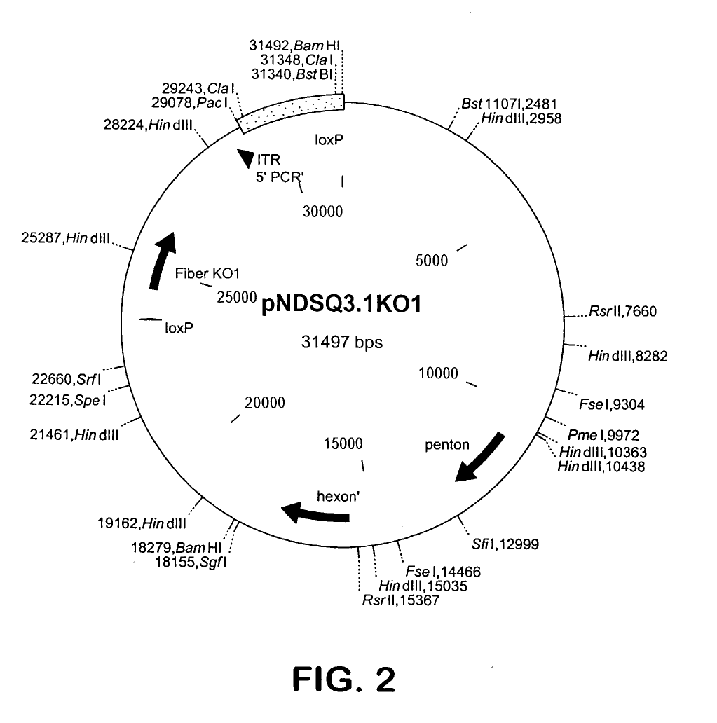

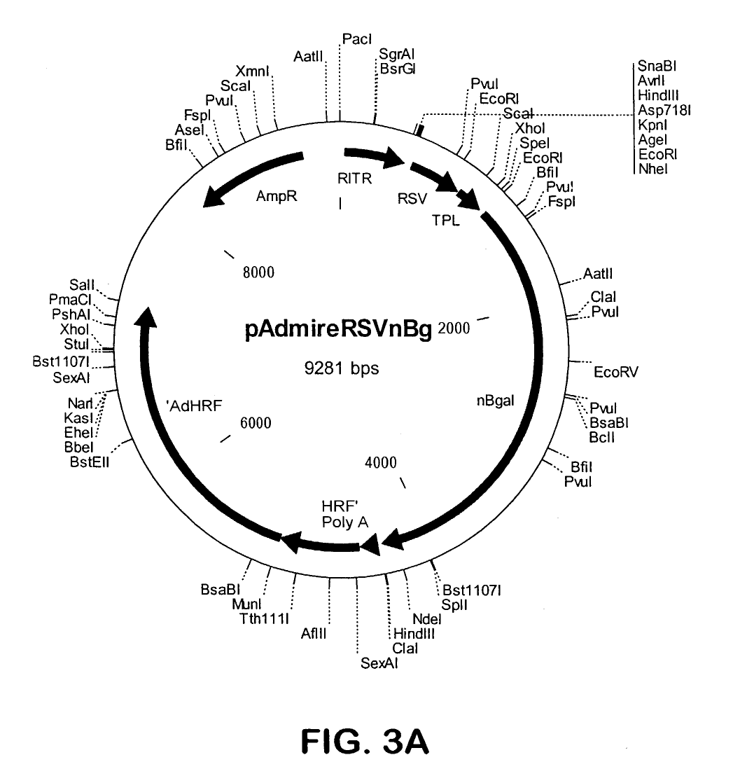

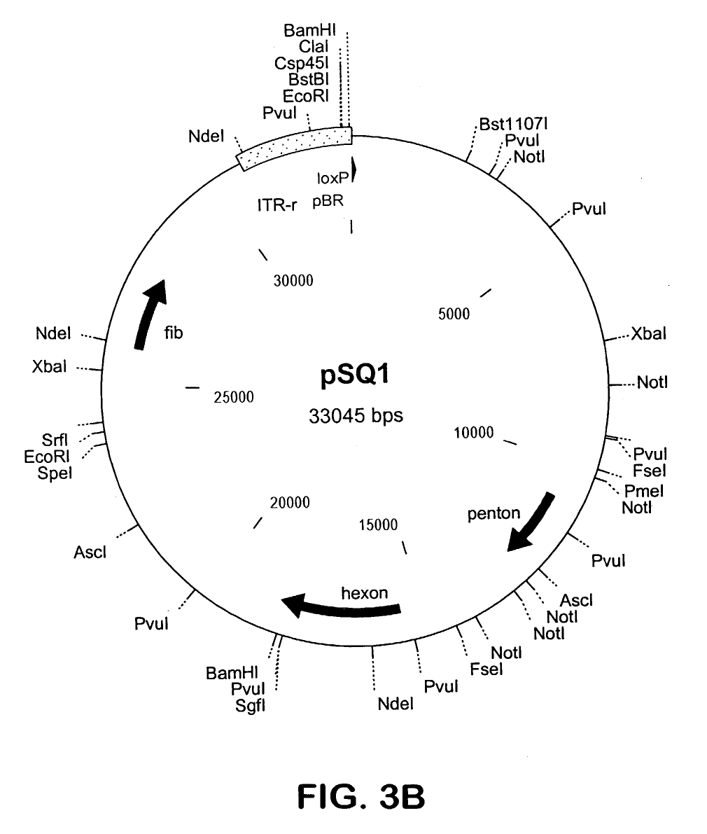

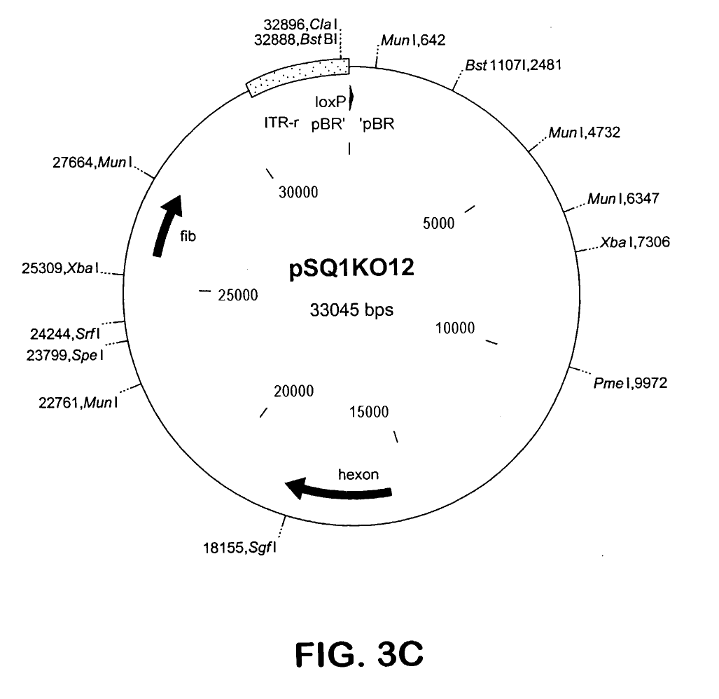

The vector was constructed as follows. First, the plasmid pSKO1 (FIG. 1) was digested with the restriction enzymes SphI and MunI. The resulting DNA fragments were separated by electrophoresis on an agarose gel. The 1601 bp fragment containing all but the 5′ end of the fiber gene was excised from the agarose gel and the DNA was isolated and purified. The fragment was then ligated with the 9236 bp fragment of p5FloxHRFRGD, which had been digested with SphI and MunI. The resulting plasmid, p5FloxHRFKO1, was digested with SpeI and PacI and the 6867 bp fragment containing the fiber gene was isolated. The fragment was ligated with the 24,630 bp SpeI-PacI fragment of pNDSQ3.1. The resulting plasmid, pNDSQ3.1KO1 (FIG. 2), was used together with pAdmireRSVnBg (FIG. 3A) to generate a plasmid which encodes the full-length adenoviral vector genome. It, however, was necessary to remove the PacI site from pNDSQ3.1KO1 (FIG. 2) prior to recombination with pAdmireRSVnBg (FIG. 3A) so that the final plasmid contains a unique PacI site adjacent to the 5′ ITR. The PacI site in pNDSQ3.1KO1 was removed by digestion with PacI followed by blunting with T4 DNA Polymerase and religation. The resulting plasmid was called pNDSQ3.1KO1 (Pac.

To generate a full-length plasmid containing the entire adenoviral genome, pAdmireRSVnBg (FIG. 3A) was digested with SalI and co-transfected into competent cells of the E. coli strain BJ5183 along with pNDSQ3.1KO1ΔPac, which had been digested with BstBI. Homologous recombination between the two plasmids generated a full-length plasmid encoding the entire adenoviral vector genome, which was called pFLAv3nBgFKO1.

The plasmid pFLAv3nBgKO1 was linearized with PacI and transfected into 633 cells. In the fiber complementing 633 cell line, the resulting viral DNA containing the KO1 mutation is capable of being packaged into infectious viral particles containing a mixture of wildtype fiber and mutant fiber proteins. After five rounds of amplification in 633 cells, a cytopathic effect was observed. Three more rounds of amplification in 633 cells were performed followed by purification of the virus by standard CsCI centrifugation procedures. This viral preparation was used to infect AE1-2a cells, which do not express fiber. The resulting virus contained only the mutant fiber protein on its capsid. Virus particles were purified by standard CsCI centrifugation procedures.

Av1nBgFKO1

The v ector Av1nBgFKO1 is made in a similar manner to Av3nBgFKO1 described above.

Av1nBgKO12

An additional fiber AB loop mutation (described by Einfeld et al. (2001) J. Virology 75:11284-11291) was incorporated into the genome of Av1nBg. This AB loop mutation is a four amino acid substitution, R512S, A515G, E516G, and K517G, and is referred to as KO12. The KO12 mutation was incorporated into the fiber gene by PCR gene overlap extension using the plasmid pSQ1 (FIG. 3B) as template. The pSQ1 plasmid contains most of the Ad5 genome, extending from base pair 3329 through the right ITR, in a pBR322 backbone. First, a segment of the Ad5 genome extending from within the E3 region into the fiber gene was amplified by PCR using the plasmid pSQ1 as a template with the following primers termed 5FF, 5′-GAA CAG GAG GTG AGC TTA GA-3′ SEQ ID No. 4), and 5FR, 5′-TCC GCC TCC ATT TAG TGA ACA GTT AGG AGA TGG AGC TGG TGT G-3′ (SEQ ID No. 6). The primer 5FR contains an 18 base 5′-extension that encodes the modified fiber AB loop amino acids from 512 through 517. A second PCR using pSQ1 as a template amplified the region immediately 3′ of the AB loop substitution and extending past the MunI site located 40 base pairs 3′ of the fiber gene stop codon. The two primers used for this reaction were 3FF: 5′-TCA CTA AAT GGA GGC GGA GAT GCT AAA CTC ACT TTG GTC TTA AC-3′ (SEQ ID No. 7), and 3FR: 5′-GTG GCA GGT TGA ATA CTA GG-3′ (SEQ ID No. 8). The primer 3FR contains an 18 base 5′-extension that encodes the modified fiber AB loop amino acids 512 through 517. Amplified products of the expected size were obtained and used in a second PCR with the end primers 5FF and 3FR to join the fragments together. The KO12 PCR fragment was digested with XbaI and MunI cloned directly into the fiber shuttle plasmid, pFBshuttle(EcoRI) to generate the plasmid pFBSEKO12 which contains the 8.8 kB EcoRI fragment of pSQ1. The pFBSEKO12 plasmid was digested with XbaI and EcoRI and cloned into pSQ1 using a three-way ligation to generate pSQ1 KO12 (FIG. 3C). The KO12 cDNA was incorporated into the genome of Av1nBg, an adenovirus vector with E1 and E3 deleted encoding β-galactosidase, by homologous recombination between ClaI-linearized pSQ1KO12 and pAdmireRSVnBg digested with SalI and PacI to generate Av1nBgKO12. The KO12 vector was transfected in 633 cells, scaled-up on non-fiber expressing cells and purified, as described above for KO1.

Av1nBgPD1

Genetic Incorporation of the PD1 Penton Mutation to Generate Av1nBgPD1

The adenoviral vector Av1nBgPD1 is an E1-, E3-deleted vector based on the adenovirus serotype 5 genome. It contains a RSV promoted nuclear-localizing β-galactosidase gene in the E1 region and also contains the PD1 mutation in the penton gene. The PD1 mutation results in a substitution of amino acids 337 through 344 of the penton protein, HAIRGDTF (SEQ ID No. 9), with amino acids SRGYPYDVPDYAGTS (SEQ ID No. 10), thus replacing the RGD tripeptide (see, Einfeld et al. (2001) J. Virology 75:11284-11291). The mutation in the penton gene was generated in the plasmid pGEMpen5, which contains the Adenovirus serotype 5 penton gene. To generate the mutation, four-oligonucleotides were synthesized. The sequences of the oligonucleotides were as follows: penton 1: 5′ CGC GGA AGA GAA CTC CAA CGC GGC AGC CGC GGC AAT GCA GCC GGT GGA GGA CAT GAA 3′ (SEQ ID No. 11); penton 2: 5′ TAT CGT TCA TGT CCT CCA CCG GCT GCA TTG CCG CGG CTG CCG CGT TGG AGT TCT CTT CC 3′ (SEQ ID No. 12); penton 3: 5′ CGA TAG CCG CGG CTA CCC CTA CGA CGT GCC CGA CTA CGC GGG CAC CAG CGC CAC ACG GGC TGA GGA GAA GCG CGC 3′ (SEQ ID No. 13); penton 4: 5′ TCA GCG CGC TTC TCC TCA GCC CGT GTG GCG CTG GTG CCC GCG TAG TCG GGC ACG TCG TAG GGG TAG CCG CGG C 3′ (SEQ ID No. 14). The complementary oligonucleotides penton 1 and penton 2 were annealed to each other as were penton 3 and penton 4. The duplex generated by annealing penton 3 and penton 4 encoded the substitution of amino acids 337 through 344 described above. The duplex generated by annealing penton 1 and penton 2 possessed a 5 base 5′ overhang which was compatible to a 5 base 5′ overhang on the duplex generated by annealing penton 3 and penton 4. The opposite end of the duplex generated by annealing penton 1 and penton 2 contained an Earl compatible overhang. The opposite end of the duplex generated by annealing penton 3 and penton 4 contained a BbvCI compatible overhang. The two duplexes were ligated to each other and ligated back into the pGEMpen5 backbone as follows. First, pGEMpen5 was digested with BbvCI and PstI and the resulting DNA fragments were separated by electrophoresis on an agarose gel. The 3360 bp fragment was excised from the gel and purified. The plasmid pGEMpen5 was also digested with PstI and EarI and the resulting fragments were separated by electrophoresis on an agarose gel. The 955 bp fragment was excised from the gel and purified. These two fragments from the pGEMpen5 plasmid were ligated with the two pairs of annealed oligonucleotides to generate the plasmid pGEMpen5PD1.

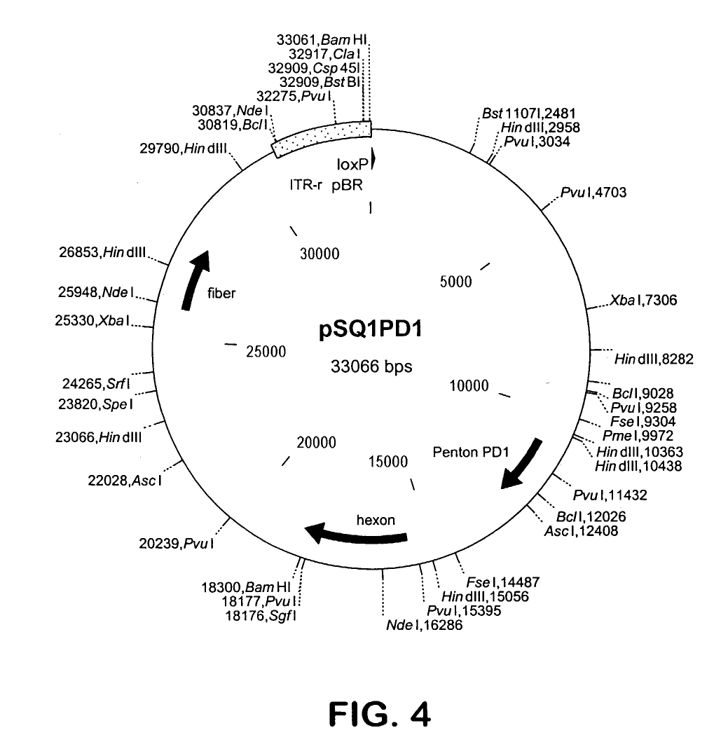

The mutated penton gene was transferred from pGEMpen5PD1 to pSQ1 using a 5-way ligation as follows. First, the region of the penton gene containing the PD1 mutation was excised from pGEMpen5PD1 by digestion with PvuI and AscI. The 974 bp fragment containing the PD1 mutation was purified. Four DNA fragments were prepared from the pSQ1 plasmid (FIG. 3B) as follows. The plasmid was digested with Csp451 and FseI and the 9465 bp fragment was purified. In addition pSQ1 was digested with FseI and PvuI and the 2126 bp fragment was purified. The plasmid pSQ1 was digested with AscI and BamHI and the 5891 bp fragment was purified. Finally, pSQ1 was digested with BamHI and Csp451 and the 14610 bp fragment was purified. The 5 purified DNA fragments were ligated to each other to form the plasmid pSQ1 PD1 (FIG. 4).

To generate adenoviral vector, pSQ1PD1 was linearized by digestion with ClaI and co-transfected into PerC6 cells with pAdmireRSVnBg (FIG. 3A) which had been digested with SalI and PacI hexadimethrine bromide was maintained in the medium at 4 μg/ml. When a cytopathic effect was observed, a crude viral lysate was further expanded on PerC6 cells. The virus was purified by standard CsCI centrifugation procedures.

Av1nBgFKO1PD1

Genetic incorporation of the fiber KO1 or KO12 mutation in combination with the penton PD1 mutation to generate

Av1nBgFKO1PD1

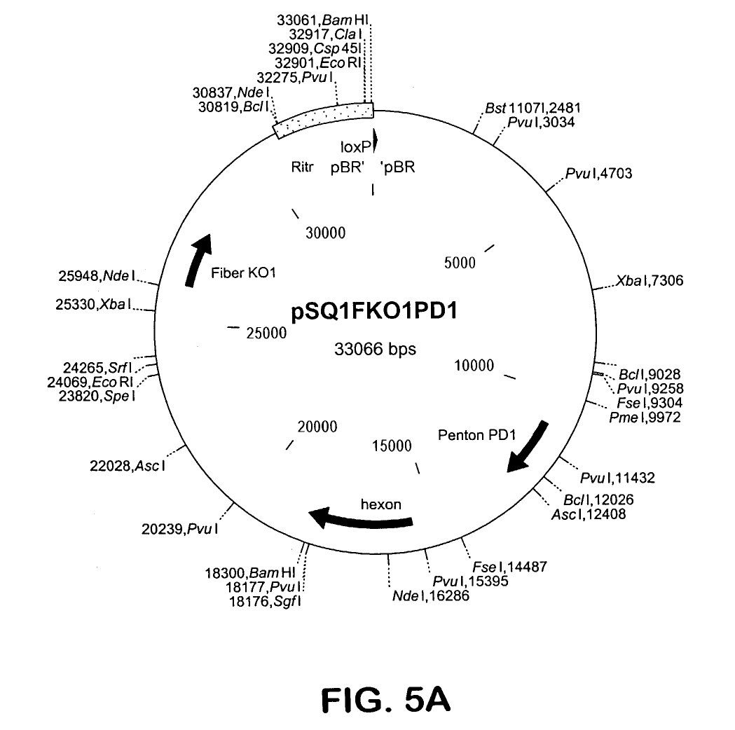

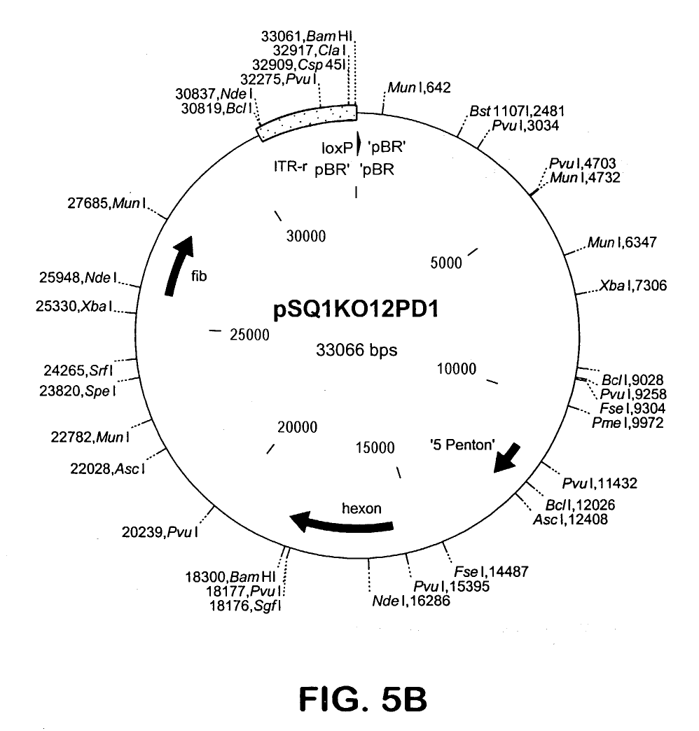

The adenoviral vectors Av1nBgFKO1PD1 and Av1nBgKO12PD1 were generated in an E1-, E3-deleted adenovirus serotype 5 genome. Both vectors contains a RSV promoted nuclear-localizing β-galactosidase gene in the E1 region and also contains either the KO1 or KO12 mutation in the fiber gene as well as the PD1 mutation in the penton gene. The vectors were constructed as follows. First, the plasmid pSQ1PD1 was digested with Csp451 and SpeI and the 23976 bp fragment containing the PD1 mutated penton gene was purified. In addition, the plasmids pSQ1 KO1 or pSQ1KO12 (FIG. 3B) were digested with Csp451 and SpeI and the 9090 bp fragment containing the KO1 or KO12 mutated fiber gene were purified. The appropriate purified fragments were ligated to each other to from the plasmid pSQ1 FKO1 PD1 (FIG. 5A) or pSQ1KO12PD1 (FIG. 5B) that contains the KO1 (or KO12) mutated fiber gene and the PD1 mutated penton gene. To generate virus, pSQ1FKO1PD1 or pSQKO12PD1 was linearized with ClaI and co-transfected into 633 cells with pAdmireRSVnBg (FIG. 3A) which had been digested with SalI and PacI. After three rounds of amplification in 633 cells a cytopathic effect was observed and the crude viral lysate was then amplified on PerC6 cells. Hexadimethrine bromide was maintained in the medium at 4 μg/ml. Each virus was purified by standard CsCI centrifugation procedures.

### EXAMPLE 2

In Vitro Evaluation of Adenoviral Vectors Containing the KO1 and PD1 Mutations

Several recombinant adenoviral vectors were used in these studies to demonstrate the function of the KO1 fiber mutation and included Av1nBg, Av1nBgFKO1, Av1nBgPD1, and Av1nBgFKO1PD1, described above. The transduction efficiencies of adenoviral vectors containing the KO1 and/or PD1 mutations were evaluated on cells of the alveolar epithelial cell line A549. The transduction efficiencies were compared to that of Av1nBg, an adenoviral vector containing wild type fiber and penton.

The day prior to infection, cells were seeded into 24-well plates at a density of approximately 1×10⁵ cells per well. Immediately prior to infection, the exact number of cells per well was determined by counting a representative well of cells. Each of the vectors, Av1nBg, Av1 nBgFKO1, and Av1 nBgFKO1 PD1 were used to transduce A549 cells at each of the following particle per cell (PPC) ratios: 100, 500, 1000, 2500, 5000, 10,000. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector.

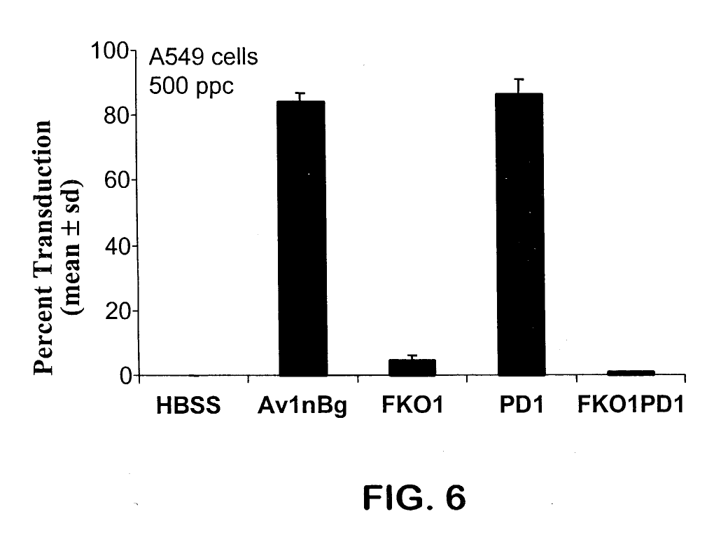

The results at the 500 PPC ratio are shown in FIG. 6 and show a significantly reduced transduction efficiency on A549 cells using vectors containing the KO1 mutation alone or when combined with PD1 compared to Av1nBg. The vectors containing the PD1 mutation alone had no effect on adenoviral transduction of A549 cells in vitro.

### EXAMPLE 3

In Vivo Analysis of Adenoviral Vectors Containing the FKO1 and PD1 Mutations

This Example provides experiments that evaluate the in vivo biodistribution of adenoviral vectors containing the KO1 and PD1 mutations and their influence on adenoviral-mediated liver transduction. The results show that ablating the viral interaction with CAR and/or integrins is not sufficient to fully detarget adenoviral vectors from the liver in vivo.

A positive control cohort received Av1nBg and a negative control group received HBSS. Additionally, the Av1nBgFKO12 and Av1nBgFKO12PD1 vectors were analyzed in vivo. These vectors each contain a fiber protein with the four amino acid substitution in the AB loop. Additionally, Av1 nBgFKO12PD1 contains a mutation in the penton base. Both of these mutations were known (see, Einfeld et al. (2001) J. Virology 75:11284-11291), and were alleged to decrease liver transduction 10 to 700 fold, respectively. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. The median lobe of the liver was placed in neutral buffered formalin to preserve the sample for β-galactosidase immunohistochemistry. In addition, tissue from each organ was frozen to preserve it for hexon PCR analysis to determine vector content. A separate sample of liver from each mouse was frozen to preserve it for a chemiluminescent β-galactosidase activity assay.

For β-galactosidase immunohistochemistry slices of liver, approximately 2-3 mm thick, were placed in 10% neutral buffered formalin. After fixation, these samples were embedded in paraffin, sectioned, and analyzed by immunohistochemistry for, β-galactosidase expression. A 1:1200 dilution was used of a rabbit anti-β-galactosidase antibody (ICN Pharmaceuticals, Inc.; Costa Mesa, Calif.) in conjunction with a Vectastain ABC kit (Vector Laboratories, Inc., Burlingame, Calif.) to visualize positive cells.

The chemiluminescent β-galactosidase activity assay was performed using the Galacto-Light Plus™ chemiluminescent assay (Tropix, Inc., Foster City, Calif.) system. Tissue samples were collected in lysis matrix tubes containing two ceramic spheres (Bio101, Carlsbad, Calif.) and frozen on dry ice. The tissues were thawed and 500 μl of lysis buffer from the Galacto-Light Plus kit was added to each tube. The tissue was homogenized for 30 seconds using a FastPrep System (Bio101, Carlsbad, Calif.). Liver samples were homogenized for an additional 30 seconds. β-galactosidase activity was determined in the liver homogenates according to the manufacture's protocol.

For hexon PCR analysis DNA from tissues was isolated using the Qiagen Blood and Cell Culture DNA Midi or Mini Kits (Qiagen Inc., Chatsworth, Calif.). Frozen tissues were partially thawed and minced using sterile disposable scalpels. Tissues were then lysed by incubation overnight at 55° C. in Qiagen buffer G2 containing 0.2 mg/ml RNaseA and 0.1 mg/ml protease. Lysates were vortexed briefly and then applied to Qiagen-tip 100 or Qiagen-tip 25 columns. Columns were washed and DNAs were eluted as described in the manufacturer's instructions. After precipitation, DNAs were dissolved in water and the concentrations were spectrophotometrically determined (A260 and A280) on a DU-600 (Beckman Coulter, Inc.; Fullerton, Calif.) or a SPECTRAmax PLUS (Molecular Devices, Inc.; Sunnyvale, Calif.) spectrophotometer. 2.3.2.

PCR primers and a Taqman probe specific to adenovirus hexon sequences were designed using Primer Express software v. 1.0 (Applied Biosystems, Foster-City, Calif.). Primer and probe sequences were: Hexon Forward primer: 5′-CTTCGATGATGCCGCAGTG-3′ (SEQ ID No. 38); Hexon Reverse primer: 5′-GGGCTCAGGTACTCCGAGG-3′ (SEQ ID No. 39); and Hexon Probe: 5′-FAM-TTACATGCACATCTCGGGCCAGGAC-TAMRA-3′ (SEQ ID No. 40).

Amplification was performed in a reaction volume of 50 μl under the following conditions: 10 ng (tumor) or 1 μg (liver and lung) of sample DNA, 1× Taqman Universal PCR Master Mix (Applied Biosystems), 600 nM forward primer, 900 nM reverse primer and 100 nM hexon probe. Thermal cycling conditions were: 2 minute incubation at 50° C., 10 minutes at 95° C., followed by 35 cycles of successive incubation at 95° C. for 15 seconds and 60° C. for 1 minute. Data was collected and analyzed using the 7700 Sequence Detection System software v. 1.6.3 (Applied Biosystems). Quantification of adenovirus copy number was performed using a standard curve that includes dilutions of adenovirus DNA from 1,500,000 copies to 15 copies in the appropriate background of cellular genomic DNA. For analysis of tumor tissues, a standard curve in a background of 10 ng human DNA was generated. For analysis of mouse liver and lung tissues, a standard curve using the same adenovirus DNA dilutions in a background of 1 μg CD-1 mouse genomic DNA was generated. Samples were amplified in triplicate, and the average number of total copies was normalized to copies per cell based on the input DNA weight amount and a genome size of 6×10⁹ bp.

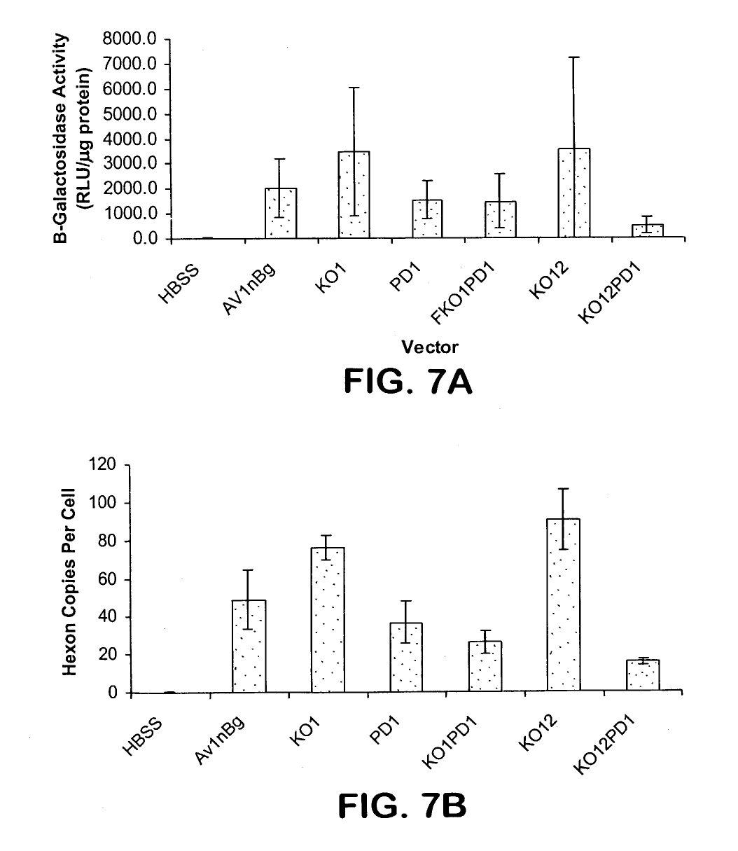

The results of the β-galactosidase activity assay and adenoviral hexon DNA content for liver transduction by these vectors are shown in FIGS. 7A and 7B. The vector containing the KO1 or KO12 mutations alone showed, on average, a slight increase in liver transduction compared to Av1nBg, which is consistent with several previous experiments. The vectors containing the PD1 mutation alone or combined with KO1 or KO12 showed a slight decrease in liver transduction compared to Av1nBg, suggesting that integrins are involved to some extent in hepatic uptake of the adenoviral vectors.

The results of the immunohistochemical staining of liver sections for β-galactosidase were consistent with the activity assays (data not shown) and demonstrate that gene expression was localized specifically to hepatocytes. The vectors containing the KO1 or KO12 mutation alone showed a slight increase in liver transduction as revealed by a more intense and frequent immunohistochemical-staining pattern. The vectors containing the PD1 mutation, either alone or combined with KO1 or KO12, showed little difference in transduction compared to Av1nBg. These results demonstrate that ablating the viral interaction with CAR and/or integrins is not sufficient to fully detarget adenoviral vectors from the liver in vivo.

In summary, the fiber AB loop mutation contained in Av1nBgFKO1 or Av1nBgKO12 ablates interaction with human and mouse CAR in vitro and diminished transduction in vitro. In vivo, however, fiber AB loop mutations behaved unexpectantly, because such mutations were found to enhance adenoviral-mediated gene transfer to liver and results in increasing vector potency. The penton base, PD1 mutation that ablates interaction with the second receptor involved in adenoviral internalization had no effect in vitro and little to no effect in vivo. These studies indicated that other receptors are responsible for adenoviral gene transfer to the liver in vivo.

### EXAMPLE 4

Description Of Adenoviral Vectors Containing A Fiber With Amino Acid Substitutions At The Heparin Sulfate Binding Domain In The Fiber Shaft

Vectors containing substitutions at all four of the amino acids in the four amino acid motif in the Ad5 fiber shaft (residues 91 to 94, KKTK; SEQ ID No. 1) were generated in order to ablate the potential interaction with HSP. The mutation is termed HSP because it potentially eliminates binding to heparan sulfate proteoglycans. Vectors containing the HSP mutation alone and combined with the KO1 mutation (fiber knob AB loop mutation that ablates CAR binding), the PD1 mutation (penton mutation that eliminates RGD/integrin interaction), and a triple knockout vector (HSP, KO1, PD1) were generated.

Generation of the HSP fiber mutation: The HSP mutation was incorporated into the fiber gene by using a PCR-based strategy of gene splicing by overlap extension (PCR SOEing). First, a segment of the Ad5 genome extending from within the E3 region into the 5′ end of the fiber gene was amplified by PCR using the plasmid pSQ1 (FIG. 3B) as a template and two primers termed 5FF and 5HSPR. The DNA sequence of 5FF is as follows: 5′ GAA CAG GAG GTG AGC TTA GA 3′ (SEQ ID No. 5). This sequence corresponds to base pairs 25,199-25,218 of pSQ1. The DNA sequence of 5HSPR is as follows: 5′ GGC TCC GGC TCC GAG AGG TGG GCT CAC AGT GGT TAC ATT T 3′ (SEQ ID No. 15). 5HSPR is a reverse primer for 5FF and corresponds to a region in the fiber shaft adjacent to the KKTK (SEQ ID No. 1) region. The primer contains a 5′ extension that encodes a GAGA substitution for the native KKTK (encoded by SEQ ID No. 1) amino acid sequence. A second PCR using pSQ1 as a template amplified the region immediately 3′ of the KKTK (SEQ ID No. 1) site and extending past the Muni site located 40 base pairs 3′ of the stop codon for the fiber gene. The two primers used for this reaction were 3HSPF and 3FR. The DNA sequence of 3HSPF is as follows: 5′ GGA GCC GGA GCC TCA AAC ATA AAC CTG GAA AT 3′ (SEQ ID No. 16). It contains a 5′ extension that is complementary to the 5′ extension of 5HSPR. The DNA sequence of 3FR is as follows: 5′ GTG GCA GGT TGA ATA CTA GG 3′ (SEQ ID No. 8).

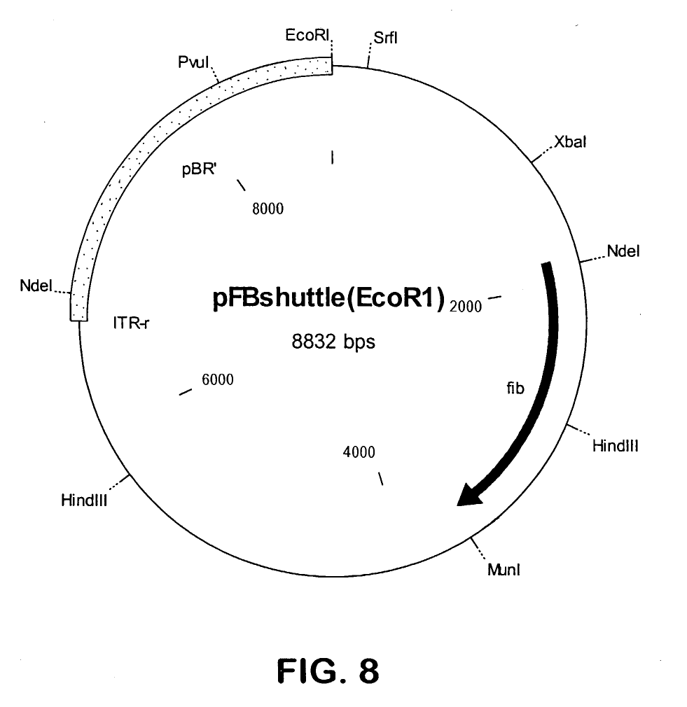

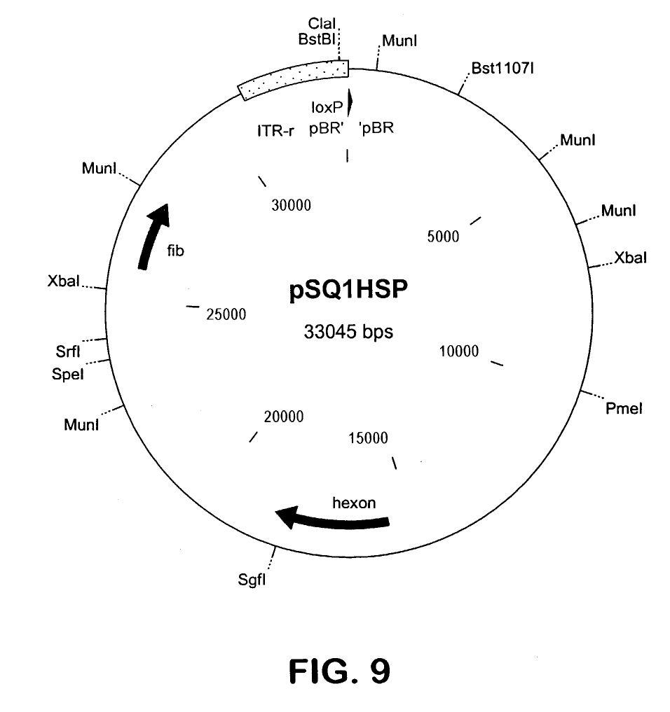

The two PCR products were joined by PCR SOEing using primers 5FF and 3FR. The resulting PCR product was digested with the restriction enzymes XbaI and MunI. The 2355 bp fragment was gel purified and ligated with the 6477 bp XbaI to MunI fragment of the plasmid pFBshuttle(EcoRI) (FIG. 8) to generate the plasmid pFBSEHSP. The plasmid pFBshuttle(EcoRI) was generated by digesting the plasmid pSQ1 with EcoRI, then gel purifying and self-ligating the 8.8 kb fragment containing the fiber gene. Next, the fiber gene containing the HSP mutation was transferred from pFBSEHSP into pSQ1 using a three-way ligation. The 16,431 bp EcoRI to NdeI fragment of pSQ1, the 9043 bp NdeI to XbaI fragment of pSQ1, and the 7571 bp XbaI to EcoRI fragment of pFBSEHSP were isolated and ligated to generate pSQ1 HSP (FIG. 9).

To generate a recombinant adenoviral vector containing the HSP mutation in the fiber gene, pSQ1 HSP was digested with ClaI and pAdmireRSVnBg (FIG. 3A) was digested with SalI and PacI, then the two digested plasmids were co-transfected into 633 cells (von Seggern et al. (2000) J Virology 74:354-362). Homologous recombination between the two plasmids generated a full-length adenoviral genome capable of replication in 633 cells, which inducibly express Ad5E1A and constitutively express wild-type fiber protein. After propagation on 633 cells, the virus capsid contained wildtype and mutant fiber proteins. To obtain viral particles containing only the modified fiber with the HSP mutation, the viral preparation was used to infect PerC6 cells, which do not express fiber. The resulting virus, termed Av1nBgFS*, was purified by standard CsCI centrifugation procedures.

Generation of Vector Containing the HSP and KO1 Mutations

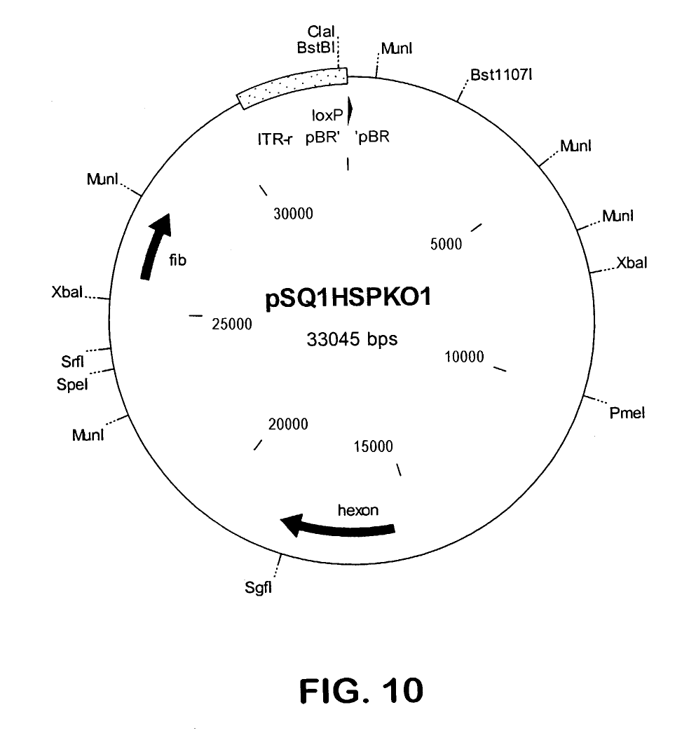

To generate an adenoviral vector containing the HSP and KO1 mutations in fiber, a PCR SOEing strategy identical to the one described above was used except that the plasmid pSQ1 FKO1 was used as the template. The PCR SOEing product was digested with XbaI and MunI and ligated with the 6477 bp XbaI to MunI fragment of pFBshuttle(EcoRI) to generate pFBSEHSPKO1. The fiber gene containing the HSP and KO1 mutations was transferred from pFBSEHSPKO1 into the pSQ1 backbone using a three-way ligation strategy identical to the one described above for the HSP mutation alone, to generate the plasmid pSQ1 HSPKO1 (FIG. 10). Recombinant adenoviral vector containing the HSP and KO1 mutations in the fiber gene was generated by co-transfecting pSQ1HSPKO1 digested with ClaI and pAdmireRSVnBg digested with SalI and PacI into 633 cells. Adenovirus was propagated and purified as described above for the vector containing the HSP mutation alone. The resulting virus was termed Av1nBgFKO1S*.

Generation of Vector Containing the HSP and PD1 Mutations

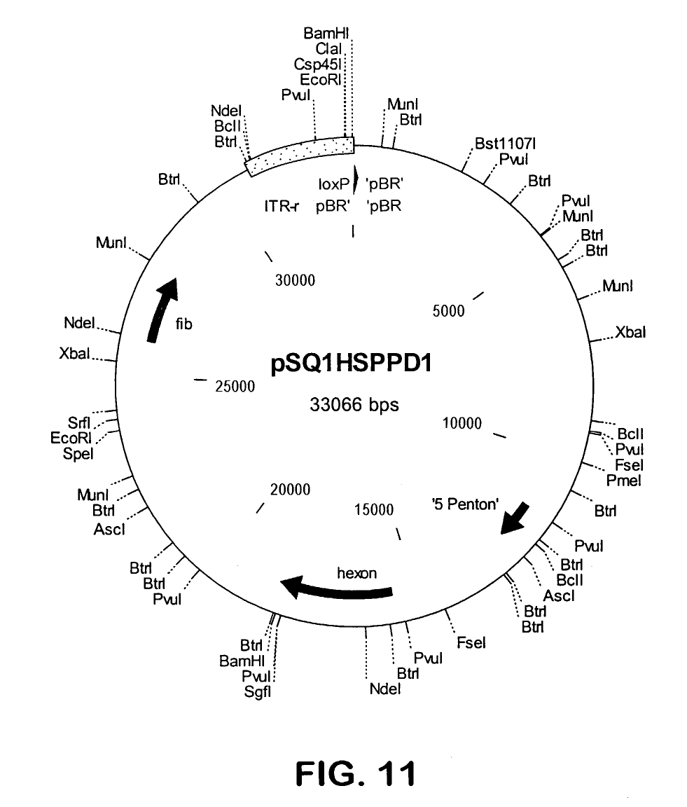

The following strategy was used to generate a recombinant adenoviral vector containing the fiber HSP mutation and the penton PD1 mutation. The plasmid pSQ1 PD1 (FIG. 4) was digested with the restriction enzymes Csp451 and SpeI and the 23,976 bp fragment was isolated and purified. In addition, the plasmid pSQ1 HSP was also digested with Csp451 and SpeI and the 9090 bp fragment was isolated and purified and ligated to the 23,976 bp fragment to generate the plasmid pSQ1 HSPPD1 (FIG. 11), which contains the fiber HSP and penton PD1 mutations. An adenoviral vector was generated, propagated, and purified as described above. The resulting virus was termed Av1nBgS*PD1.

Generation of Vector Containing the HSP, KO1, and PD1 Mutations

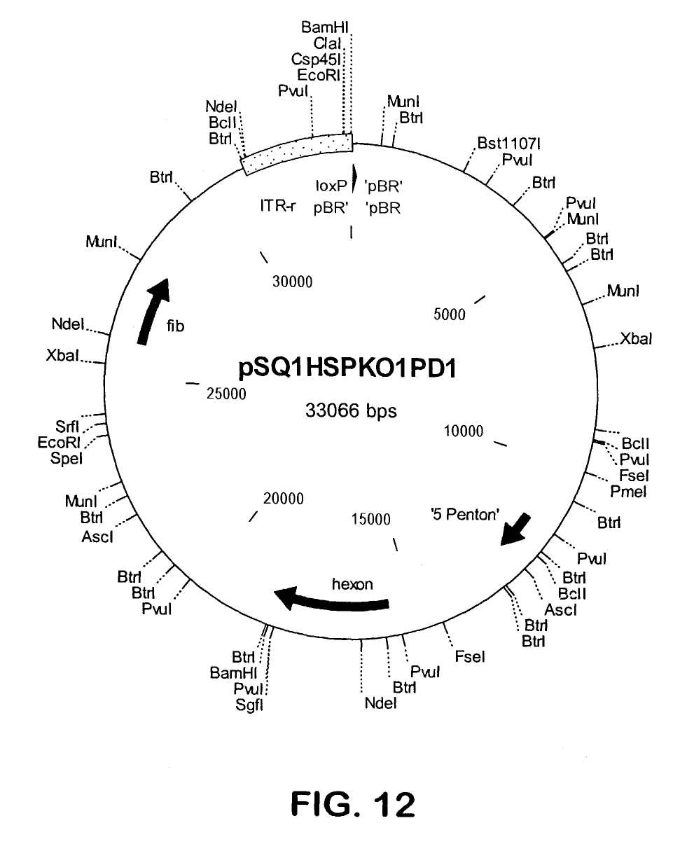

To generate an adenoviral vector containing the HSP, KO1, and PD1 mutations the following strategy was used. First, the plasmid pSQ1PD1 was digested with Csp451 and SpeI and the 23,976 bp fragment was isolated and purified. In addition, the plasmid pSQ1HSPKO1 was digested with Csp451 and SpeI and the 9090 bp fragment was isolated and purified. The two DNA fragments were ligated to form the plasmid pSQ1HSPKO1PD1 (FIG. 12). Recombinant adenoviral vector was generated, propagated, and purified as described above. The resulting virus was termed Av1nBgFKO1S*PD1.

### EXAMPLE 5

In Vitro Evaluation Of Adenoviral Vectors Containing The HSP Fiber Mutation

The transduction efficiencies of adenoviral vectors containing the HSP mutation in the fiber gene, either alone or combined with the KO1 and/or PD1 mutations, were evaluated on A549 and HeLa cells. The transduction efficiencies were compared to that of Av1nBg, an adenoviral vector containing wild type fiber and penton. The day prior to infection, cells were seeded into 24-well plates at a density of approximately 1×10⁵ cells per well. Immediately prior to infection, the exact number of cells per well was determined by counting a representative well of cells. Each of the vectors, Av1nBg (see, Stevenson et al. (1997) J. Virol. 71:4782-4790), Av1nBgS*, Av1nBgFKO1S*, Av1nBgS*PD1, and Av1nBgFKO1S*PD1, were used to transduce A549 cells at each of the following particle per cell (PPC) ratios: 100, 500, 1000, 2500, 5000, 10,000. HeLa cells were transduced with each of the above vectors, as well as a vector containing the KO1 mutation alone (Av1nBgFKO1) and a vector containing the PD1 mutation alone (Av1nBgPD1) at 2000 PPC. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector.

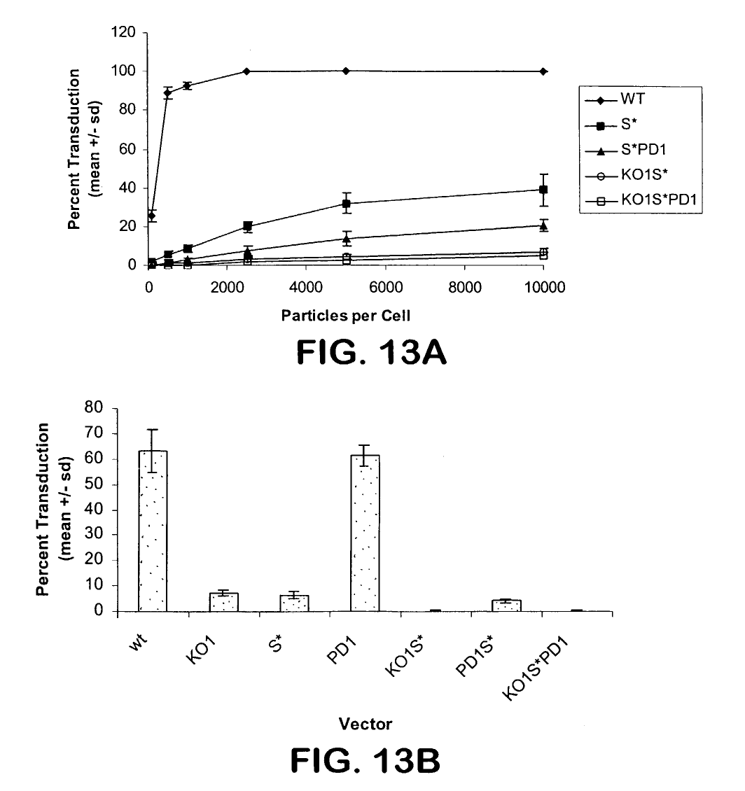

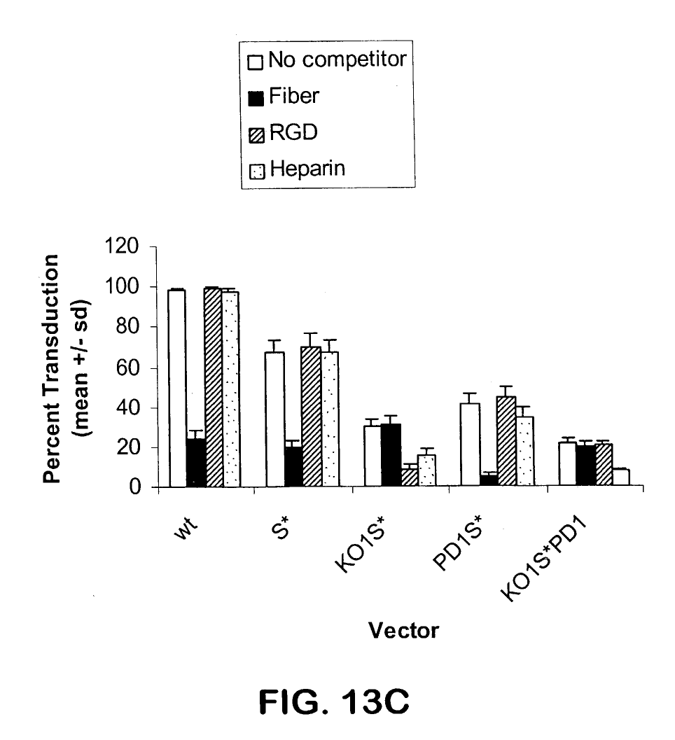

The results (depicted in FIGS. 13A-13B) showed significantly reduced transduction efficiencies on A549 and HeLa cells using vectors containing the HSP mutation compared to Av1nBg. The vectors containing the HSP mutations, however, demonstrated a dose response on A549 cells, in that increasing PPC ratios yielded increasing transduction.

Competition experiments were done to determine which receptor molecular interactions are involved in transduction of A549 cells by the various vectors. Transductions were performed in the presence or absence of various competitors including Ad5 fiber knob, a 50 amino acid oligopeptide derived from Adenovirus serotype 2 penton base which spans the RGD tripeptide region, or heparin (Invitrogen Life Technologies, Gaithersburg, Md.). Monolayers of A549 cells were cultured in Richters medium supplemented with 10% FBS and were transduced with Av1nBg, Av1nBgS*, Av1nBgFKO1S*, Av1nBgS*PD1, or Av1 nBgFKO1S*PD1 in infection medium (IM, Richters medium plus 2% FBS). Different PPC ratios were used for the different vectors to achieve measurable transduction levels. The PPC ratios were as follows: Av1nBg: 500 PPC, Av1nBgS*: 10,000 PPC, Av1nBgFKO1S*: 20,000 PPC, Av1nBgS*PD1: 10,000 PPC, and Av1nBgFKO1S*PD1: 20,000 PPC. Fiber knob competition was performed by pre-incubating cells in IM containing 16 μg/ml of fiber knob for 10 minutes at room temperature prior to infection with virus. Penton base peptide competition was performed by pre-incubating cells in IM containing 500 nM peptide for 10 minutes at room temperature prior to infection with virus. Heparin competition was performed by pre-incubating each adenoviral vector in IM containing 3 mg/ml of heparin for 20 minutes at room temperature. In all cases, the competitor remained in the IM during the 1 hour infection when virus was rocked on the cell monolayers at 37° C. in 5% CO2. After infection, the monolayers were washed with PBS, 1 ml of complete medium was added per well and the cells were incubated for an additional 24 hours to allow for β-galactosidase expression. The cell monolayers were then fixed and stained with X-Gal. The percentage of cells transduced was determined by light microscopy as described above. Each condition was carried out in triplicate and three random fields per well were counted, for a total of nine fields per condition. The average percentage of transduction per high-power field was determined.

The results of the competition experiment (FIG. 13C) showed that fiber knob inhibited transduction of cells by all vectors except for those that contained the KO1 mutation. The penton base peptide only inhibited transduction by Av1nBgFKO1S*. Heparin inhibited transduction by Av1nBgFKO1S* and Av1nBgFKO1S*PD1, but did not affect transduction by any of the other viruses suggesting the presence of additional heparin binding sites on the adenoviral capsid but that the shaft contains the predominant site.

### EXAMPLE 6

In Vivo Analysis Of Adenoviral Vectors Containing The HSP Mutation In Fiber

The objective of this study was to evaluate the in vivo biodistribution of adenoviral vectors containing the HSP mutation and to determine whether this shaft modification influences adenoviral-mediated liver transduction. In addition, vectors containing the HSP mutation combined with KO1, or PD1, or a combination of all three mutations were evaluated as well as vectors containing the KO1 mutation alone and the PD1 mutation alone. A positive control cohort received Av1nBg and a negative control group received HBSS. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. The median lobe of the liver was placed in neutral buffered formalin to preserve the sample for β-galactosidase immunohistochemistry. In addition, tissue from each organ was frozen to preserve it for hexon real time PCR analysis to determine vector content. A separate sample of liver from each mouse was frozen to preserve it for a chemiluminescent β-galactosidase activity assay. β-galactosidase immunohistochemistry, hexon real-time PCR and the chemiluminescent β-galactosidase activity assay were carried out as described in Example 3.

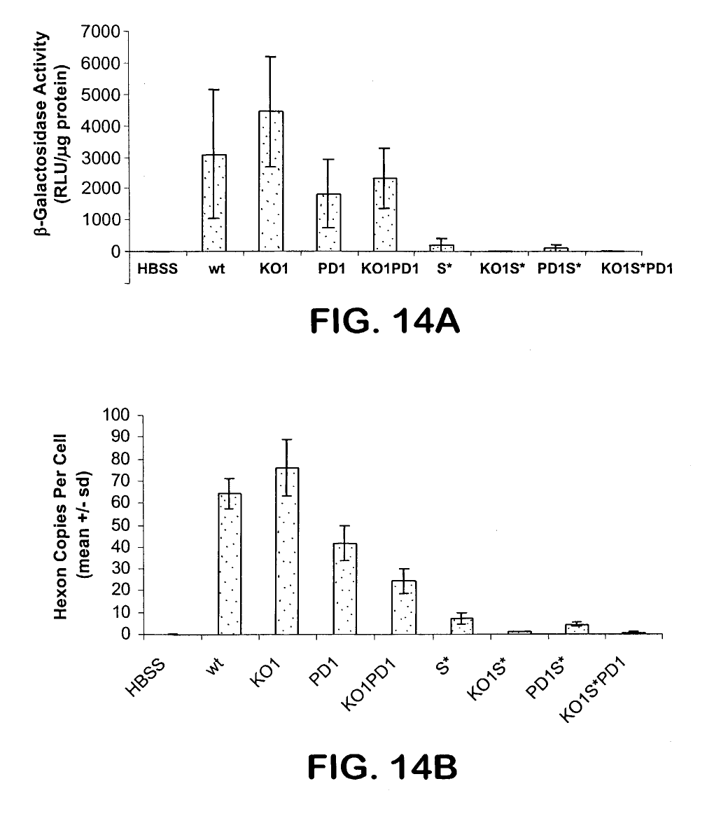

The results of the β-galactosidase activity assay (FIG. 14A) and adenoviral hexon DNA content (FIG. 14B) showed a dramatic reduction in liver transduction by vectors containing the HSP mutation. The vectors containing the HSP mutation alone resulted in reducing adenoviral-mediated liver gene expression by approximately 20-fold. When combined with the Ko1 mutation (HSP, KO1, PD1), yielded approximately a 1000-fold reduction in β-galactosidase activity in the liver compared to the control vector Av1nBg. The vector containing the KO1 mutation alone showed a slight increase, on average, in liver transduction compared to Av1nBg, which is consistent with several previous experiments. The vectors containing the PD1 mutation alone or combined with KO1 showed a slight decrease in liver transduction compared to Av1nBg, although the decrease was not statistically significant. Analysis of hepatic adenoviral hexon DNA content (FIG. 14B) confirmed these results.

The results of the immunohistochemical staining of liver sections for β-galactosidase were consistent with the activity assays (data not shown) and demonstrated that gene expression was localized specifically to hepatocytes. Vectors containing the HSP mutation, either alone or in combination with KO1 and/or PD1, showed a dramatic reduction in hepatocyte transduction. The vector containing the KO1 mutation alone showed a slight increase in liver transduction as revealed by a more intense and frequent immunohistochemical staining pattern. The vectors containing the PD1 mutation, either alone or combined with KO1, showed little difference in transduction compared to Av1nBg.

### EXAMPLE 7

Description of Adenoviral Vectors Containing the HSP Fiber Shaft Mutation With and Without the KO1 Fiber Mutation and With and Without a cRGD Targeting Ligand in the Fiber Knob HI Loop

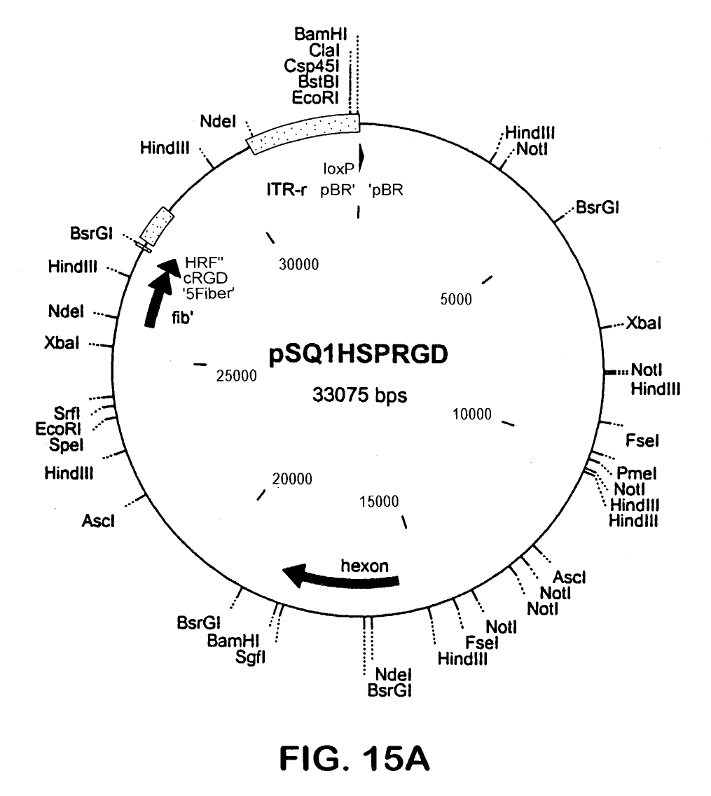

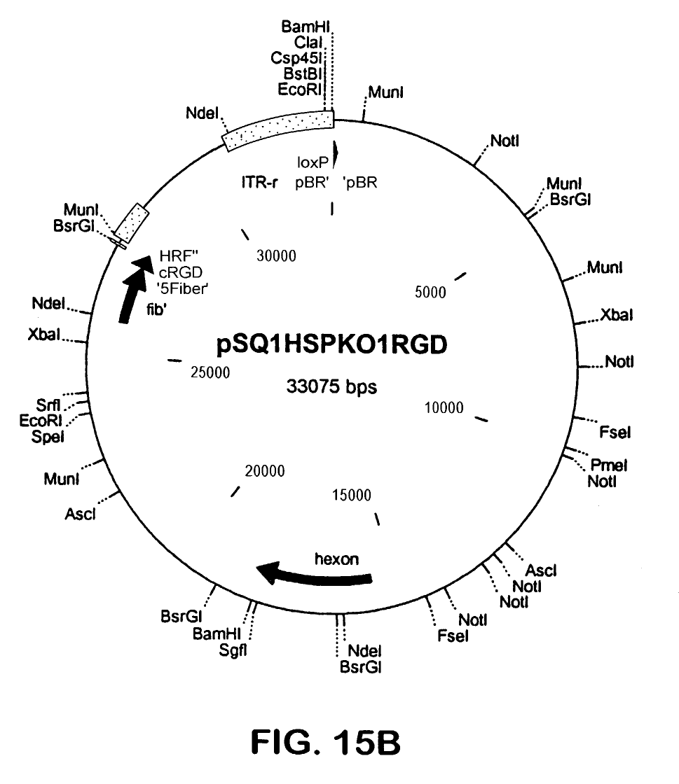

Generation of vector containing the HSP fiber shaft mutation and a cRGD ligand in the HI loop: The following strategy was used to generate an adenoviral vector containing a fiber with the HSP shaft mutation and a cRGD ligand in the HI loop. The plasmid p5FloxHRFRGD was digested with the restriction enzymes BstXI and KpnI and the 1157 bp fragment was isolated and purified. In addition, the fiber shuttle plasmid pFBSEHSP, described in Example 1 above, was digested with BstXI and KpnI and the 4549 bp and 3156 bp fragments were isolated and purified. The three fragments were ligated to generate the plasmid pFBSEHSPRGD, which encodes a fiber containing the HSP mutation and cRGD in the Hi loop. The fiber gene from this plasmid was transferred into the pSQ1 backbone as follows. The plasmid pFBSEHSPRGD was digested with EcoRI and XbaI and the 7601 bp fragment was isolated and purified. The plasmid pSQ1 (FIG. 3B) was digested with the restriction enzymes EcoRI, NdeI, and XbaI and the 16,431 bp EcoRI to NdeI fragment and the 9043 bp NdeI to XbaI fragment were isolated and purified. The three DNA fragments were ligated to generate the plasmid pSQ1 HSPRGD (FIG. 15A).

To generate a recombinant adenoviral vector containing the HSP mutation in the fiber gene along with a cRGD ligand in the HI loop, the plasmid pSQ1 HSPRGD was digested with ClaI and co-transfected into 633 cells with pAdmireRSVnBg which had been digested with SalI and PacI. After propagation on 633 cells, the virus capsid contained wildtype and mutant fiber proteins. To obtain viral particles containing only the modified fiber with the HSP mutation and a cRGD ligand, the viral preparation was used to infect PerC6 cells, which do not express fiber. The resulting virus, termed Av1nBgS*RGD, was purified by standard CsCI centrifugation procedures.

Generation of Vector Containing the HSP Fiber Shaft Mutation, the KO1 Fiber Knob Mutation, and a cRGD Ligand in the HI Loop

The following strategy was used to generate an adenoviral vector containing a fiber with the HSP shaft mutation, the KO1 fiber knob mutation, and a cRGD ligand in the HI loop. The plasmid p5FloxHRFRGD was digested with the restriction enzymes BstXI and KpnI and the 1157 bp fragment was isolated and purified. In addition, the fiber shuttle plasmid pFBSEHSPKO1, described in Example 1 above, was digested with BstXI and KpnI and the 4549 bp and 3156 bp fragments were isolated and purified. The three fragments were ligated to generate the plasmid pFBSEHSPKO1 RGD, which encodes a fiber containing the HSP mutation, the KO1 mutation, and cRGD in the HI loop. The fiber gene from this plasmid was transferred into the pSQ1 backbone as follows. The plasmid pFBSEHSPKPO1 RGD was digested with EcoRI and XbaI and the 7601 bp fragment was isolated and purified. The plasmid pSQ1 (FIG. 3B) was digested with the restriction enzymes EcoRI, NdeI, and XbaI and the 16,431 bp EcoRI to NdeI fragment and the 9043 bp NdeI to XbaI fragment were isolated and purified. The three DNA fragments were ligated to generate the plasmid pSQ1 HSPKO1 RGD (FIG. 15B).

To generate a recombinant adenoviral vector containing the HSP and KO1 mutations in the fiber gene along with a cRGD ligand in the HI loop, the plasmid pSQ1HSPKO1RGD was digested with ClaI and co-transfected into 633 cells with pAdmireRSVnBg which had been digested with SalI and PacI. After propagation on 633 cells, the virus capsid contained wildtype and mutant fiber proteins. To obtain viral particles containing only the modified fiber with the HSP and KO1 mutations and a cRGD ligand, the viral preparation was used to infect PerC6 cells, which do not express fiber. The resulting virus, termed Av1nBgFKO1S*RGD, was purified by standard CsCI centrifugation procedures.

### EXAMPLE 8

In Vitro Evaluation of Adenoviral Vectors Containing the HSP Fiber Shaft Mutation With or Without the Fiber Knob KO1 Mutation and With or Without a cRGD Ligand in the HI Loop

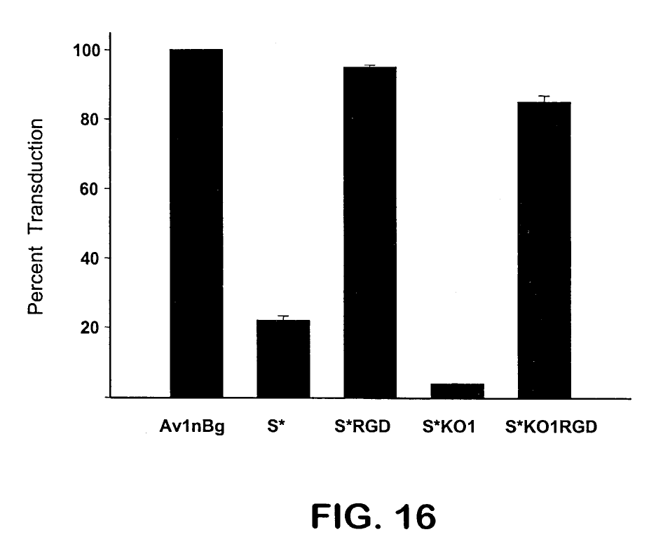

The transduction efficiencies of adenoviral vectors containing the HSP fiber shaft mutation with or without the fiber KO1 mutation and with or without the cRGD ligand in the HI loop were evaluated on A549 cells. The transduction efficiencies were compared to that of Av1nBg, an adenoviral vector containing wild type fiber. The day prior to infection, cells were seeded into 24-well plates at a density of approximately 1×10⁵ cells per well. Immediately prior to infection, the exact number of cells per well was determined by counting a representative well of cells. Each of the vectors, Av1nBg, Av1nBgS*, Av1nBgFKO1S*, Av1nBgS*RGD, and Av1nBgFKO1S*RGD, were used to transduce A549 cells at a particle to cell ratio of 6250. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector. The results (FIG. 16) showed that the cRGD ligand dramatically increased the transduction efficiencies of vectors containing the HSP mutation alone or combined with the KO1 mutation. Av1nBgS* yielded approximately 22% positive cells, while Av1nBgS*RGD yielded approximately 95% positive cells. Similarly, Av1nBgFKo1S* yielded only 4% positive cells, while Av1nBgFKO1S*RGD yielded 85% positive cells. Therefore, the vector containing the shaft mutation is viable and can be retargeted with the addition of a ligand.

### EXAMPLE 9

Construction of Ad5 Vectors Containing the Ad35 Fiber and Derivatives Thereof

The KO1 and HSP mutations in the Ad5 fiber protein (5F), described above, were designed to ablate interactions that are responsible for the normal tropism of the Ad5 virus. An alternative strategy to detarget the virus is to replace the Ad5 fiber with a fiber from another serotype which does not bind CAR and which does not possess the heparin sulfate proteoglycan (HSP) binding domain (KKTK; SEQ ID No. 1) within the shaft. The fiber of adenovirus serotype 35 (35F) does not bind CAR and does not possess the HSP binding domain in its shaft. Replacement of the 5F with the 35F can detarget the liver and provide a suitable platform for retargeting the vector to the desired tissue.

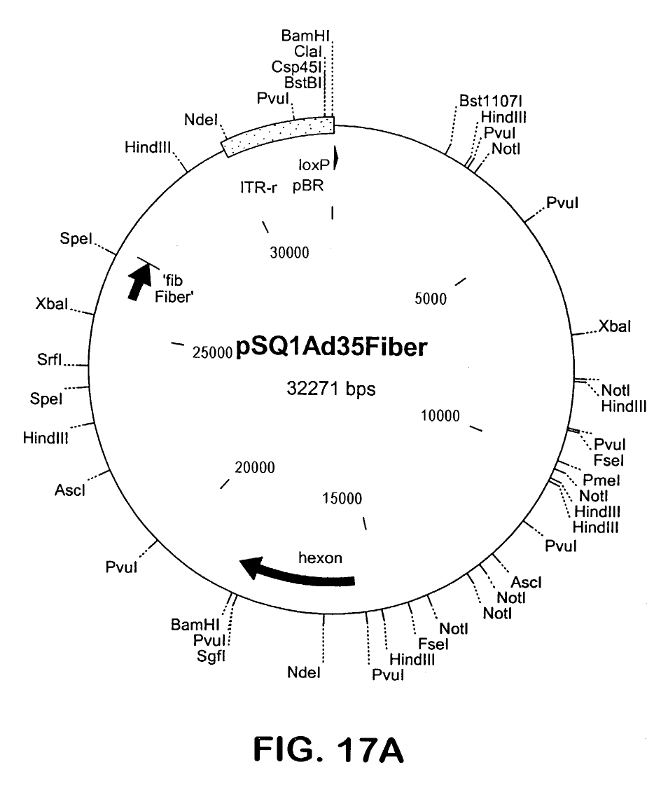

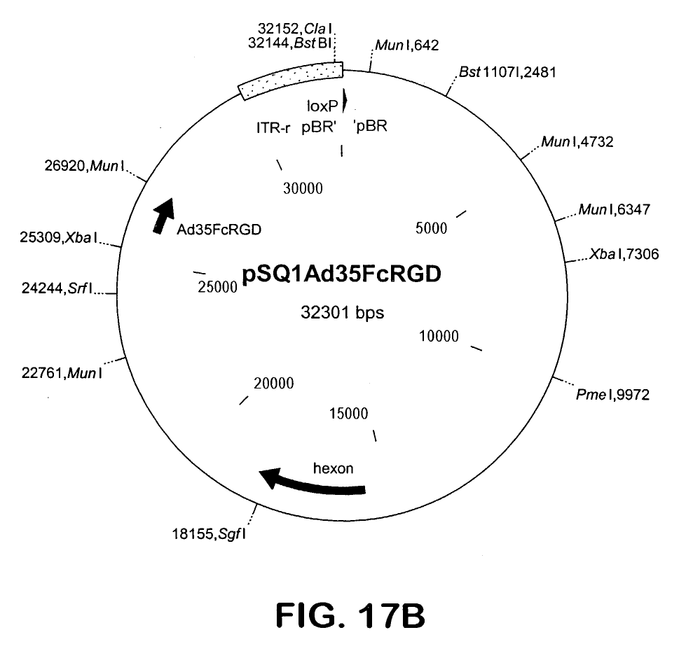

Generation of an Ad5 based vector containing the Ad35 fiber: A PCR SOEing strategy was used to generate a vector based on the Ad5 serotype but containing the Ad35 fiber in place of the Ad5 fiber. First, PCR was used to amplify a region in the plasmid pSQ1 between the Xbal site at bp 25,309 and the start of the fiber gene. The primers used for this reaction were P-0005/U and P-0006/L. The DNA sequence of P-0005/U was as follows: 5° C. TCT AGA AAT GGA CGG AAT TAT TAC AG 3′ (SEQ ID No. 17). This sequence corresponds to bp 25,308 through 25,334 of pSQ1. The DNA sequence of P-0006/L was as follows: 5′ TCT TGG TCA TCT GCA ACA ACA TGA AGA TAG TG 3′ (SEQ ID No. 18). It contains a 10 base pair 5′ extension that is complementary to the start of the Ad35 fiber gene, while the remainder of the primer anneals to the sequence immediately 5′ of the ATG start codon of the fiber gene in pSQ1. A PCR product of the expected size, 583 bp, was obtained and the DNA was gel purified. A second PCR amplified the Ad35 fiber gene using DNA extracted from wildtype Ad35 virus as a template. The primers used for this reaction were P-0007/U and 35FMun. The DNA sequence of P-0007/U was as follows: 5′ GT TGT TGC AG ATG ACC AAG AGA GTC CGG CTC A 3′ (SEQ ID No. 19). It contains a 10 base pair 5′ extension that is homologous to the 10 bp immediately prior to the ATG start codon of the fiber gene in Ad5. The remainder of the primer anneals to the start of the Ad35 fiber gene. The DNA sequence of 35FMun was as follows: 5′ AG CAA TTG AAA AAT AAA CAC GTT GAA ACA TAA CAC AAA CGA TTC TTT A GTT GTC GTC TTC TGT AAT GTA AGA A 3′ (SEQ ID No. 20). It contains a 46 base pair 5′ extension that is complementary to the region of the Ad5 genome between the end of fiber and the MunI site 40 bp downstream of the fiber gene. In addition, the 5′ extension encodes the last amino acid and stop codon of the Ad5 fiber gene. This region was retained in the vector because it contains the polyadenylation site for the fiber gene. The remainder of the primer anneals to the 3′ end of the Ad35 fiber gene, up to the next to last amino acid codon. A PCR product of the expected size, 1027 bp, was obtained and the DNA was gel purified. The two PCR products were mixed and joined together by PCR SOEing using primers P-0005/U and P-0009. The DNA sequence of P-0009 was as follows: 5′ AG CAA TTG AAA AAT AAA CAC GTT G 3′ (SEQ ID No. 21). It corresponds to bp 27,648 through 27,669 of pSQ1 and overlaps the MunI site in that region. A PCR product of the expected size, 1590 bp, was obtained and gel purified. It was cloned into the plasmid pCR4blunt-TOPO (Invitrogen Corporation, Carlsbad Calif.) using the Zero Blunt TOPO PCR Cloning Kit from Invitrogen. This intermediate cloning step simplified DNA sequencing of the PCR SOEing product. The resulting plasmid, termed pTOPOAd35F, was digested with XbaI and MunI and the 1585 bp digestion product was gel purified and ligated with the 6477 bp fragment of pFBshuttle (EcoRI) digested with XbaI and MunI to generate the plasmid pFBshuttleAd35F. The Ad35 fiber gene was transferred from pFBshuttleAd35F into pSQ1 as follows. The plasmid pSQ1 was digested with EcoRI and the 24,213 bp fragment was gel purified. The plasmid pFBshuttleAd35F was linearized with EcoRI and ligated with the 24,213 bp fragment from pSQ1. Restriction diagnostics were performed to screen for constructs containing the Ad35 fiber gene inserted into the pSQ1 backbone in the correct orientation. The pSQ1 plasmid containing the Ad35 fiber gene in the proper orientation was termed pSQ1Ad35Fiber (FIG. 17A). To generate adenoviral vector containing the Ad35 fiber, pSQ1Ad35Fiber was digested with ClaI and co-transfected into 633 cells with pAdmireRSVnBg which had been digested with SalI and PacI. After propagation on 633 cells, the resulting virus contained Ad5 fiber and Ad35 fibers on its capsid. The virus was amplified on PerC6 cells to generate virus containing only the Ad35 fiber on its capsid. The resulting virus preparation was termed Av1nBg35F.

Construction of adenoviral vectors containing chimeric fibers derived from Ad5 and Ad35: Two chimeric fiber constructs were prepared by PCR gene overlap extension using plasmids containing the full length Ad5 or Ad35 fiber cDNAs as templates. The Ad5 fiber tail and shaft regions (5TS; amino acids 1 to 403) were connected with the Ad35 fiber head region (35H; amino acids 137 to 323) to form the 5TS35H chimera, and the Ad35 fiber tail and shaft regions (35TS; amino acids 1 to 136) were connected with the Ad5 fiber head region (5H; amino acids 404 to 581) to form the 35TS5H chimera. The fusions were made at the conserved TLWT sequence at the fiber shaft-head junction.

For the construction of the 5TS35H chimera, the pFBshuttle(EcoRI) plasmid was used as the template with primers P1 and P2 to generate the 5′ fragment. The 3′ fragment was generated using the pFBshuttleAd35 plasmid as the template with the P3 and P4 primers. The sequence of each primer used in the construction of these chimeric fibers is listed in Table 2. Amplified PCR products of the expected size were obtained and were gel purified. A second PCR was carried out with the end primers P1 and P4 to join the two fragments together. The DNA fragment generated in the second PCR was digested with Xba1 and Mun1 and was cloned directly into pFBshuttle (EcoRI) to create the fiber shuttle plasmid pFBshuttle5TS35H.

TABLE 2

Primers Used For The Exchange Of Fiber Shaft Re-
gions Between Ad5 And Ad35 Fibers

| | | |
|---|---|---|
| Primer | | SEQ |
| designation | Sequence | ID |

| | | |
|---|---|---|
| P1 | 5′-GAACAGGAGGTGAGCTTAGA-3′ | 22 |
| P2 | 5′-GTTAGGTGGAGGGTTTATTCCGGTCCAC | 23 |
| | AAAGTTAGCTTATC-3′ | |
| P3 | 5′-GATAAGCTAACTTTGTGGACCGGAATAAA | 24 |
| | CCCTCCACCTAAC-3′ | |
| P4 | 5′-GTGGCAGGTTGAATACTAGG-3 | 25 |
| P5 | 5′-GTTAGGAGATGGAGCTGGTGTAGTCCATA | 26 |
| | AGGTGTTAATAC-3′ | |
| P6 | 5′-GTATTAACACCTTATGGACTACACCAGCT | 27 |
| | CCATCTCCTAAC-3′ | |
| P7 | 5′-TGCGCAAAAACAATCACCACGACAATCACAAT | 28 |
| | GTACATTGGAAGAAATCATACG-3′ | |
| P8 | 5′-ACATTGTGATTGTCGTGGTGATT | 29 |
| | GTTTTTGCGCATATGCCATACAATTTGAATG-3′ | |

For the construction of the 35TS5H chimera, the pFBshuttleAd35 plasmid was used as the template with the P1 and P5 primers to generate the 5′ fragment. The 3′ fragment was generated using the pFBshuttle (EcoRI) plasmid as the template with the P6 and P4 primers. Following the same procedure described above, the fiber shuttle plasmid pFBshuttle35TS5H was generated.

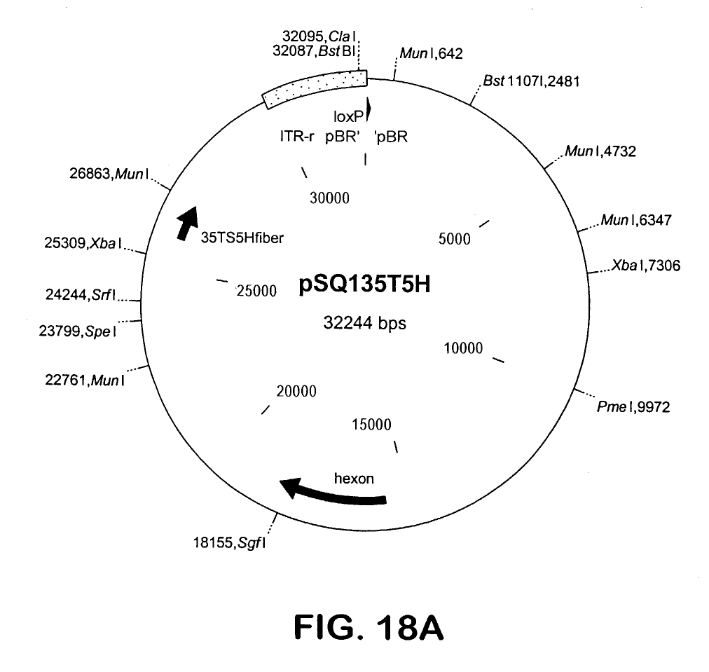

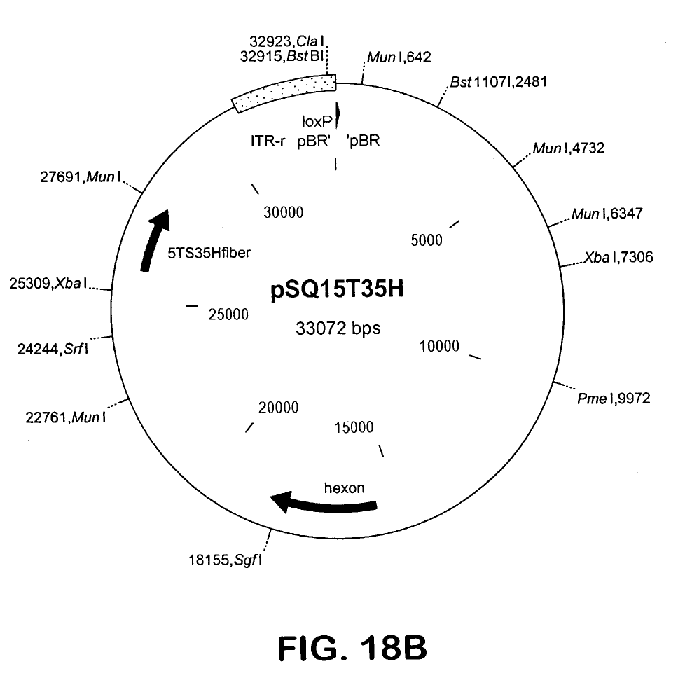

For the 35TS5H and 5TS35H chimeras, the fiber gene was transferred from the pFBshuttle(EcoRI) backbone into pSQ1 as described above for the vector containing the Ad35 fiber. The resulting plasmids were called pSQ135T5H (FIG. 18A) and pSQ15T35H (FIG. 18B). In addition, adenoviral vectors were generated using the co-transfection strategy described above.

Construction of Ad5 vectors containing the Ad35 fiber with a cRGD targeting peptide in the HI loop of the 35F fiber knob: To incorporate the cRGD targeting peptide into the Ad35 fiber HI loop, the P7 and P8 oligonucleotide primers encoding the ten amino acid sequence HCDCRGDCFC (SEQ ID No. 30) were synthesized. The pFBshuttleAd35 plasmid containing the full length Ad35 fiber cDNA was used as the template in the PCR reaction with the P1 and P7 primer pair or with the P4 and P8 primer pair in order to generate the 5′ and 3′ PCR fragments. A second PCR was then carried out with the end primers P1 and P4 to join the two fragments together. The resulting PCR fragment was digested with XbaI and MunI and was cloned into pFBshuttle (EcoRI) to create the fiber shuttle plasmid pFBshuttleAd35cRGD. The modified Ad35 fiber gene was transferred into pSQ1 using the EcoRI cloning strategy described above to generate pSQ1Ad35FcRGD (FIG. 17B). Adenoviral vector was generated using the co-transfection strategy described above.

### EXAMPLE 10

In Vitro Evaluation of Adenoviral Vectors Containing 35F and Derivatives Thereof

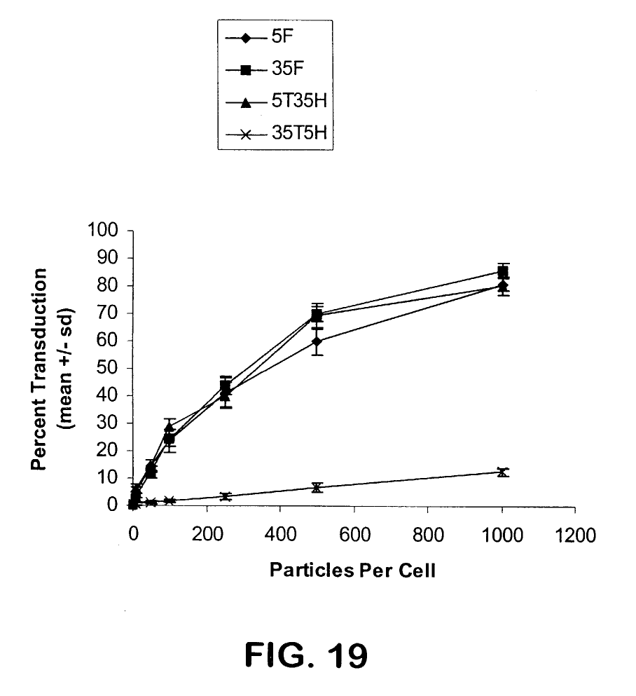

The transduction efficiencies of adenoviral vectors containing the 35F or derivatives thereof were evaluated on A549 cells. The transduction efficiencies were compared to that of Av1nBg, an adenoviral vector containing the 5F fiber. The day prior to infection, cells were seeded into 24-well plates at a density of approximately 1×10⁵ cells per well. Immediately prior to infection, the exact number of cells per well was determined by counting a representative well of cells. Each of the vectors, Av1nBg, Av1nBg35F, Av1nBg5T35H and Av1nBg35T5H were used to transduce A549 cells from 0 up to 1,000 particle per cell (PPC) ratios. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector. The results (FIG. 19) showed similar transduction efficiencies on A549 cells using the Av1nBg35F and Av1nBg5T35H vectors compared to Av1nBg. The Av1nBg35T5H showed much lower transduction efficiencies on A549 cells compared to Av1nBg as a result of the Ad35 shaft domain. The Ad35 shaft domain does not contain a HSP binding motif and the Av1nBg35T5H vector behaves similarly to the Av1nBgS* vector in vitro and in vivo. These studies also demonstrate that vectors containing fiber proteins without an HSP binding site are fully viable.

### EXAMPLE 11

In Vivo Evaluation of Adenoviral Vectors Containing 35F and Derivatives Thereof

The objective of this study was to evaluate the in vivo biodistribution of adenoviral vectors containing 35F fibers and derivatives thereof to determine whether vectors containing these fibers ablate liver transduction due to their shaft regions. A positive control cohort received Av1nBg and a negative control group received HBSS. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. The median lobe of the liver was placed in neutral buffered formalin to preserve the sample for β-galactosidase immunohistochemistry. In addition, tissue from each organ was frozen to preserve it for hexon PCR analysis to determine vector content. A separate sample of liver from each mouse was frozen to preserve it for a chemiluminescent β-galactosidase activity assay. β-galactosidase immunohistochemistry, hexon real-time PCR and the chemiluminescent β-galactosidase activity assay were carried out as described in example 3.

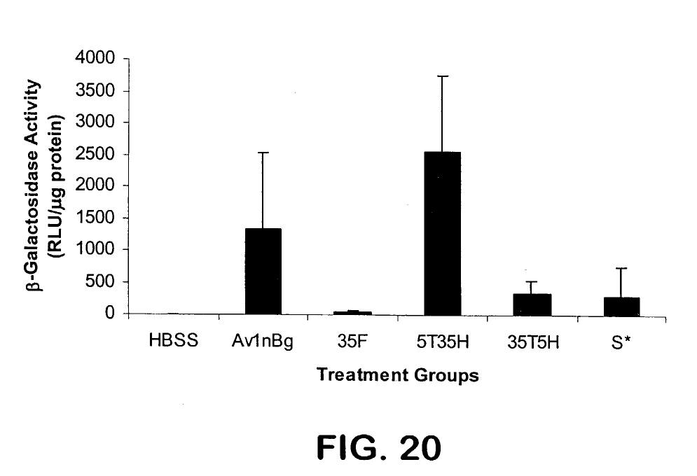

The results of the β-galactosidase activity assay showed a dramatic reduction in liver transduction by vectors containing the Ad35 fiber or the 35T5H derivative (FIG. 20) with an approximately 4- to 24-fold reduction in β-galactosidase activity in the liver compared to the control vector Av1nBg. These data demonstrate that shaft domains without HSP binding sites can effectively ablate hepatic in vivo gene transfer. In particular, HSP is the major entry mechanism for liver in vivo. CAR binding is a minor entry pathway.

### EXAMPLE 12

Construction of Ad5 Vectors Containing the Ad Serotype 41 Short Fiber and Derivatives Thereof

The human adenovirus serotype 41 contains two different fibers on its capsid, encoded by two adjacent genes. One fiber has a molecular weight of 60 kDa and is approximately 315A in length and is termed the long fiber. The other fiber has a molecular weight of 40 kDa and is approximately 250+ in length and is termed the short fiber. The Ad41 short fiber does not bind CAR and does not possess the heparin binding domain (KKTK) in its shaft. Therefore, this fiber provides a useful platform for adenoviral vector targeting.

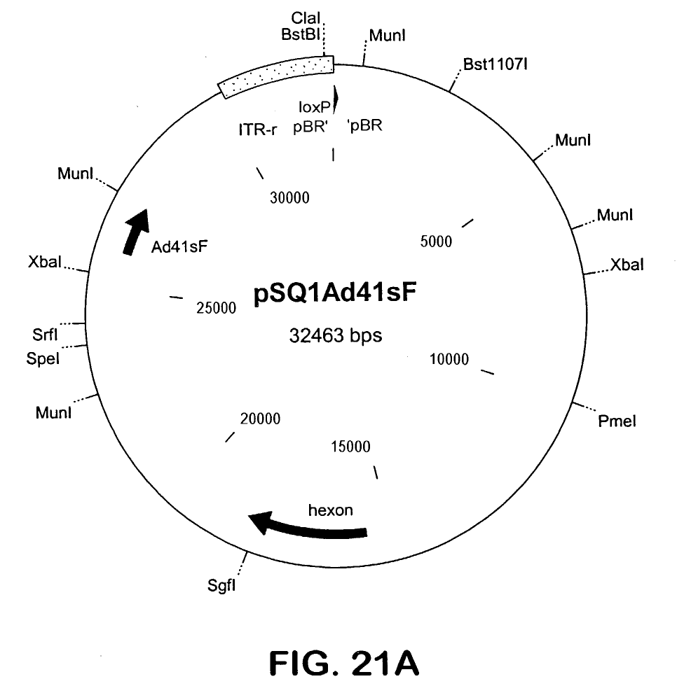

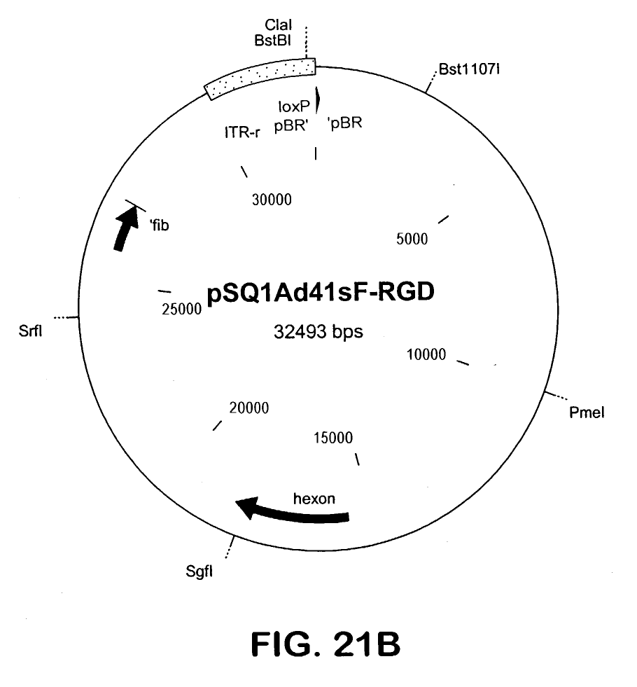

Construction of adenoviral vectors based on Ad5 but containing the Ad41 short fiber: A PCR SOEing strategy was used to generate a vector based on the Ad5 genome but containing the Ad41 short (Ad41s) fiber. First, PCR was used to amplify the region of pSQ1 between the XbaI site at bp 25,309 and the start of the fiber gene. The primer pair used for the PCR were P-0005/U and P-0010/L. The DNA sequence of P-0005/U was as follows: 5° C. TCT AGA AAT GGA CGG AAT TAT TAC AG 3′ (SEQ ID No. 17). The sequence corresponds to bp 25,308 through 25,334 of pSQ1 and overlaps the XbaI site in that region. The DNA sequence of P-0010/L was as follows: 5′ TTC TTT TCA T CTG CAA CAA CAT GAA GAT AGT G 3′ (SEQ ID No. 31). It contains a 5′ extension corresponding to the first 10 bp of the Ad41s fiber gene. The remainder of the primer anneals to pSQ1 immediately 5′ of the ATG start codon of the fiber gene. The PCR product was the expected size (583 bp). A second PCR was used to amplify the Ad41s fiber using the plasmid pDV60Ad41sF as a template. The primers used were P-0011/U and P-0012/L. The DNA sequence of P-0011/U was as follows: 5′ GT TGT TGC AG ATG AAA AGA ACC AGA ATT GAA G 3′ (SEQ ID No. 32). It contains a 10 bp 5′ extension corresponding to the DNA sequence immediately 5′ of the ATG start codon of the fiber gene in pSQ1. The remainder of the primer anneals to the beginning of the Ad41s fiber gene in pDV60Ad41sF. The DNA sequence of P-0012/L was as follows: 5′ TG CAA TTG AAA AAT AAA CAC GTT GAA ACA TAA CAC AAA CGA TTC TTT ATT C TTC AGT TAT GTA GCA AAA TAC A 3′ (SEQ ID No. 33). It contains a 51 bp 5′ extension corresponding to the sequence in pSQ1 from the last codon of the fiber gene through the MunI site 40 bp downstream of the fiber gene. The remainder of the primer anneals to the 3′ end of the Ad41s fiber gene in pDV60Ad41 sF. The PCR product was the expected size (1219 bp). The two PCR products were joined by PCR SOEing using primers P-0005/U and P-0009/L. The DNA sequence of P-0009/L was described above. The PCR SOEing reaction yielded the expected 1782 bp product. The product was cloned into pCR4blunt-TOPO to yield pCR4blunt-TOPOAd41 sF. Next, pCR4blunt-TOPOAd41 sF was digested with XbaI and MunI and the 1773 bp fragment containing the Ad41s fiber gene was gel purified. This fragment was ligated with the 6477 bp XbaI to MunI fragment of pFBshuttle(EcoRI) to generate pFBshuttleAd41sF. The Ad41s fiber gene was transferred into the pSQ1 backbone as follows. First, pFBshuttleAd41sF was linearized using EcoRI and this fragment was ligated with the 24,213 bp EcoRI fragment of pSQ1 to generate pSQ1Ad41sF (FIG. 21A). Adenoviral vector containing the Ad41s fiber was generated using the co-transfection strategy described above.

Construction of Ad5 adenoviral vectors containing the Ad41 short fiber with a cRGD targeting ligand in the HI loop: A PCR SOEing strategy was used to generate a construct containing the Ad41s fiber with cRGD in the HI loop. The plasmid pFBshuttleAd41sF was used as a template for the PCR amplifications. First, a 1782 bp fragment was amplified using primers 5FF and 41sRGDR. The primer 5FF was described above. It anneals to pFBshuttleAd41sF at the XbaI site upstream of the fiber gene. The DNA sequence of the primer 41 sRGDR was as follows: 5′ AGT ACA AAA ACA ATC ACC ACG ACA ATC ACA GTT TAT CTC GTT GTA GAC GAC ACT GA 3′ SEQ ID No. 34). It contains a 30 bp 5′ extension that encodes the cRGD targeting ligand. The remainder of the primer anneals to pFBshuttleAd41sF from bp 2878 through 2903. A second PCR amplified a 277 bp region of pFBshuttleAd41sF using primers 3FR and 41sRGDF. The primer 3FR was described previously. It anneals to pFBshuttleAd41sF at the MunI site downstream of the fiber gene. The DNA sequence of 41 sRGDF was as follows: 5′ TGT GAT TGT CGT GGT GAT TGT TTT TGT ACT AGT GGG TAT GCT TTT ACT TTT 3′ (SEQ ID No. 35). It contains a 30 bp 5′ extension that encodes the cRGD targeting ligand and is complementary to the extension on 41 sRGDR. The remainder of the primer anneals to pFBshuttleAd41sF from bp 2904 through 2924. The two PCR products were joined by PCR SOEing to generate a 2059 bp fragment using primers 5FF and 3FR. The product was digested with XbaI and MunI and the 1803 bp DNA fragment was gel purified. The fragment was ligated with the 6477 bp fragment resulting from digestion of pFBshuttle(EcoRI) with XbaI and MunI. The resulting plasmid was termed pFBshuttleAd41sRGD. This plasmid was linearized by EcoRI digestion and ligated with the 24,213 bp EcoRI fragment of pSQ1 to generate pSQ1Ad41sRGD (FIG. 21B).

### EXAMPLE 13

In Vivo Evaluation Of Ad5 Vectors Containing the Ad41 Short Fiber and Derivatives Thereof

This example evaluates the in vivo biodistribution of adenoviral vectors containing 41sF fibers and derivatives thereof to determine whether vectors containing the these fibers ablate liver transduction due to modified shaft regions. A positive control cohort received Av3nBg (see, Gorziglia et al. (1996) J. Virology 70:4173-4178) or Ad5.βGal.ΔF/5F, and a negative control group received HBSS. Ad5.βGal.ΔF/5F is a derivative of the fiberless vector Ad5.βgal.ΔF (ATCC accession number VR2636) modified to express AD5 fiber (see, e.g., International PCT application No. WO0183729).

The Ad5.βGal.ΔF vector was pseudotyped with the Ad41sF fiber protein and injected in vivo. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. The median lobe of the liver was placed in neutral buffered formalin to preserve the sample for β-galactosidase immunohistochemistry. In addition, tissue from each organ was frozen to preserve it for hexon PCR analysis to determine vector content. A separate sample of liver from each mouse was frozen to preserve it for a chemiluminescent β-galactosidase activity assay. β-galactosidase immunohistochemistry, hexon real-time PCR and the chemiluminescent β-galactosidase activity assay was carried out as described in example 3.

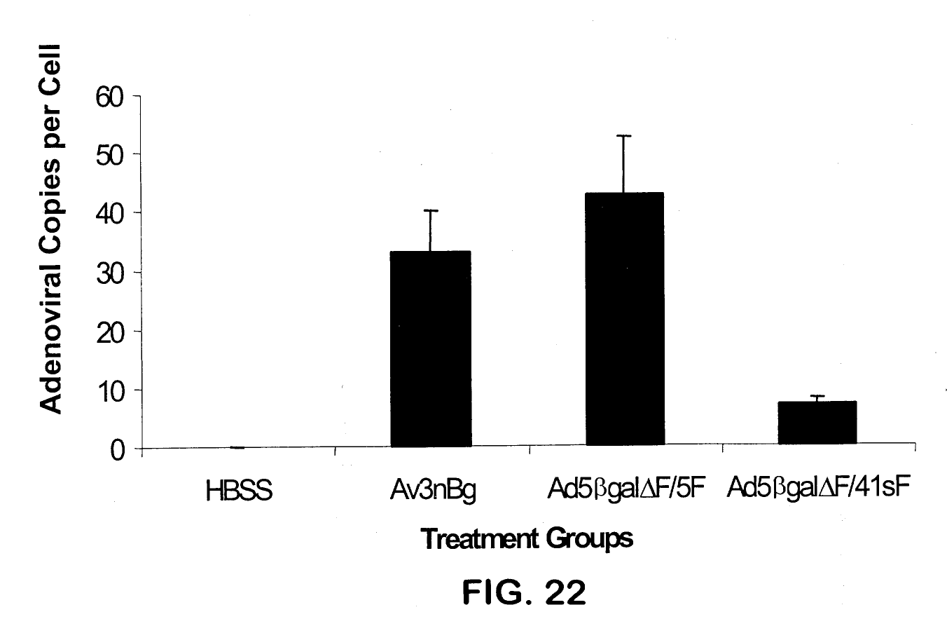

The results of the hexon DNA analysis showed a dramatic reduction in liver transduction by vectors containing the Ad41sF fiber (FIG. 22) with an approximately a 5-fold reduction in liver adenoviral DNA content compared to either control vector.

In the above examples, several novel adenoviral vectors were generated containing various fiber modifications designed to ablate the normal tropism of the vector. See Table 3. Vectors were generated in which the heparan sulfate binding domain in the fiber shaft was replaced by amino acid substitutions. This mutation, termed HSP, was also combined with the Ko1 mutation (fiber knob AB loop mutation that ablates CAR binding), and the PD1 mutation (penton mutation that eliminates RGD/integrin interaction). In addition, a vector containing all three mutations (HSP, KO1, PD1) was generated. All vectors containing the HSP mutation, either alone or combined with other capsid modifications, showed dramatically reduced transduction efficiencies on A549 and HeLa cells. Furthermore, the same vectors showed dramatically reduced transduction of the liver following systemic delivery to mice. As an alternative strategy to ablate the normal tropism of Ad5-based vectors, the Ad5 fiber was replaced by a fiber from a different adenovirus serotype which does not bind CAR and does not contain the heparan binding domain in the shaft. Thus, vectors were generated containing the Ad35 fiber and the Ad41 short fiber. Versions of these two vectors containing a cRGD targeting ligand in the HI loop of the fiber were also produced. Additionally, vectors containing chimeric fibers were generated. A vector containing the Ad35 fiber tail and shaft regions fused to the Ad5 fiber knob domain as well as a vector containing the Ad5 fiber tail and shaft fused to the Ad35 fiber knob domain were constructed. Vectors containing either the entire Ad35 or Ad41 short fiber showed a significant reduction in liver transduction following delivery to mice via the tail vein. The observation of reduced liver transduction using vectors containing either an HSP mutation, the Ad35 fiber, or the Ad41 short fiber indicates the feasibility of detargeting adenoviral vectors in vivo. In vitro data with the Ad35 fiber or the Ad41 short fiber with cRGD (see Example 14) indicate that the virus is completely viable, that is, it is not damaged by the absence of an HSP binding site and is retargetable. Taken together these data suggest that these vectors provide a suitable platform for retargeting strategies.

TABLE 3
Description Of Recombinant Adenoviral Vectors Used
To Demonstrate That Shaft Modifications Influence
Tropism In Vivo Vector

| Vector | Description |
|---|---|
| Av1nBg | An E1 and E3-deleted adenoviral vector encoding a nuclear |
| | localizing β-galactosidase |
| Ad5 Fiber derivatives: | |
| Av1nBgFKO1 | The same as Av1nBg but containing the KO1 AB loop |
| | mutation in the fiber gene |
| Av1nBgPD1 | The same as Av1nBg but containing the penton PD1 mutation |
| | that deletes the integrin binding, RGD tripeptide |
| Av1nBgS* | The same as Av1nBg but containing the 4 amino acid |
| | substitution in the shaft referred to as S* that modifies the HSP |
| | binding motif |
| Av1nBgFKO1S* | The same as Av1nBg but containing the fiber KO1 and S* |
| | mutations combined |
| Av1nBgS*PD1 | The same as Av1nBg but containing the fiber S* and penton |
| | PD1 mutations combined |
| Av1nBgFKO1S*PD1 | The same as Av1nBg but containing the fiber KO1, S* and |
| | penton PD1 mutations combined |
| Ad35 fiber derivatives: | |
| Av1nBg35F | The same as Av1nBg but containing the full length Ad35 fiber |
| | cDNA |
| Av1nBg5T35H | The same as Av1nBg but containing the 5T35H chimeric fiber |
| Av1nBg5T35H | The same as Av1nBg but containing the 35T5H chimeric fiber |
| Av1nBg35FRGD | The same as Av1nBg but containing the full length Ad35 fiber |
| | cDNA with a cRGD ligand in the HI loop of the Ad35 fiber |
| Ad41 sF fiber derivatives: | |
| Av1nBg41sF | The same as Av1nBg but containing the full length Ad41 short |
| | fiber cDNA |
| Av1nBg41sFRGD | The same as Av1nBg but containing the full length Ad41 short |
| | fiber cDNA with a cRGD ligand in the HI loop of the Ad41 |
| | short fiber |

### EXAMPLE 14

In Vitro Evaluation of Adenoviral Vectors Containing the Ad41sF With a cRGD Ligand in the HI Loop

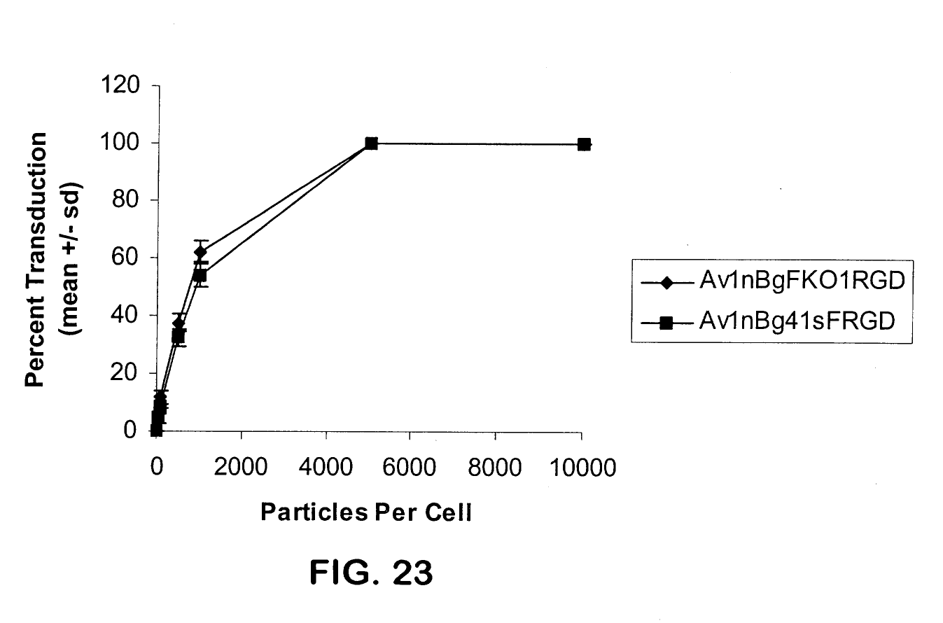

The transduction efficiencies of adenoviral vectors containing the Ad41sF fiber with the cRGD ligand in the HI loop were evaluated on A549 cells. The transduction efficiencies were compared to that of Av1nBg, an adenoviral vector containing wild type fiber or Av1nBgFKO1RGD, an adenoviral vector containing the KO1 mutation in combination with the cRGD ligand in the HI loop. The day prior to infection, cells were seeded into 24-well plates at a density of approximately 1×10⁵ cells per well. Immediately prior to infection, the exact number of cells per well was determined by counting a representative well of cells. Each of the vectors, Av1nBg, Av1nBgFKO1RGD, and Av1nBg41sFRGD were used to transduce A549 cells at a particle to cell ratios of 0 up to 10,000. The cell monolayers were stained with X-gal 24 hours after infection and the percentage of cells expressing β-galactosidase was determined by microscopic observation and counting of cells. Transductions were done in triplicate and three random fields in each well were counted, for a total of nine fields per vector. The results (FIG. 23) show that the Av1nBg41sFRGD vector transduced cells to an equivalent level as Av1nBgFKO1RGD at all vector doses examined. Neither FKO1 or Ad41sF can bind CAR. The Ad41sF does not normally interact with CAR and additionally does not contain the HSP binding motif within the shaft domain. These data show that targeting peptides inserted into the loop regions of the fiber knob of KO1 and Ad41sF allows for transduction of target cells via the targeted receptor. Surprisingly, HSP, not CAR and integrins, is the major entry route in vivo and ablation of HSP binding permits targeting of adenoviral vectors.

### EXAMPLE 15

Effect of the Shaft Modification on the Biodistribution of Adenoviral Vectors In Vivo

The influence of fiber and penton modifications on the in vivo biodistribution of adenoviral vectors containing fiber head, shaft and penton mutations was examined. Vectors containing the HSP mutation combined with KO1, or PD1, or a combination of all three mutations were evaluated as well as vectors containing the KO1 mutation alone and the PD1 mutation alone. The indicated adenoviral vectors were systemically administered to C57BL6 mice as described above. A positive control cohort received Av1nBg and a negative control group received HBSS. Cohorts of five C57BL/6 mice received each vector via tail vein injection at a dose of 1×10¹³ particles per kg. The animals were sacrificed approximately 72 hours after vector administration by carbon dioxide asphyxiation. Liver, heart, lung, spleen, and kidney were collected from each animal. Tissue from each organ was frozen to preserve it for real time PCR analysis to determine adenoviral hexon DNA content. A separate sample of liver from each mouse was frozen to preserve it for a chemiluminescent β-galactosidase activity assay. Hexon real-time PCR and the chemiluminescent β-galactosidase activity assay was carried out as described in Example 3.

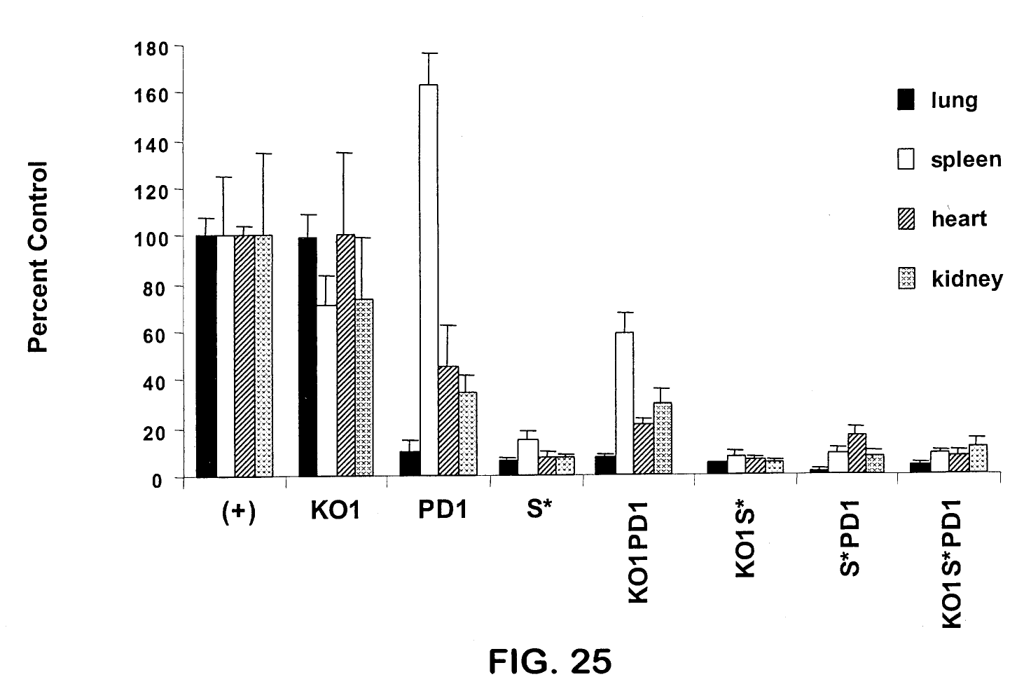

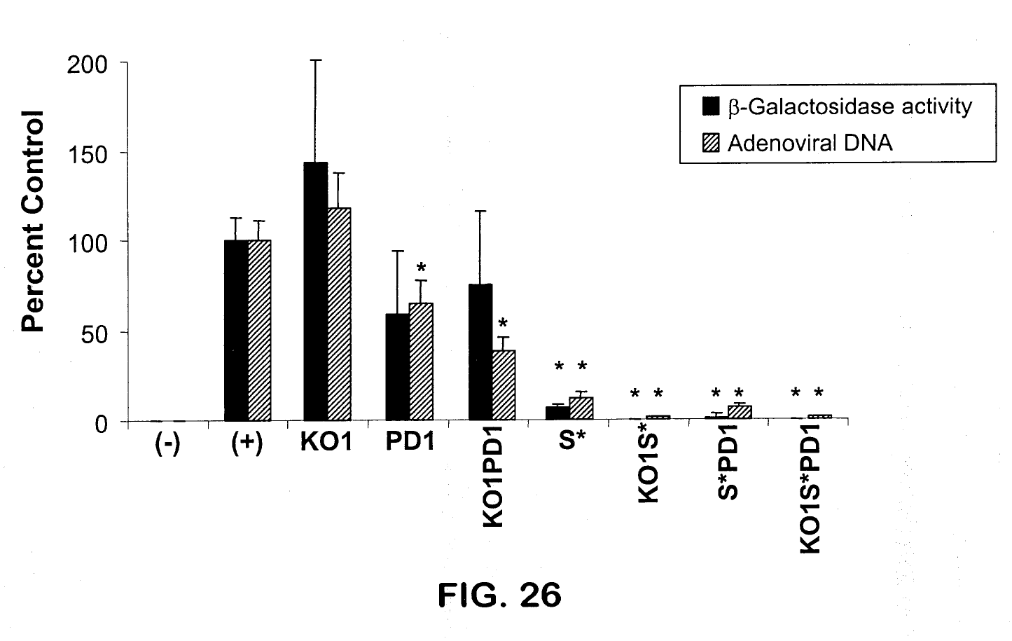

The results derived from the liver are described in Example 6 (FIGS. 14A and B) and also shown in FIG. 26 with results presented as percent control of Av1nBg. The effect of the S* shaft modification on the biodistribution of adenovirus to the other organs is shown in FIG. 25. The average adenoviral DNA content was determined as adenoviral genomic copies per cell and expressed as a percentage of the Av1nBg (+) control value. The average percent control value+standard deviation is shown (n=5 per group) for each tissue examined (FIG. 25).

Systemic delivery of Ad5 based vectors with wild-type fiber results in a preferential accumulation of vector DNA in the liver with 64 copies per cell with significantly less DNA found in the other organs with 1.32 copies per cell found in lung, 2.18 copies per cell in spleen, 0.47 copies per cell found in heart, and 0.72 copies per cell in the kidney. All differences found with PD1, S*, KO1PD1, KO1S*, S*PD1, and KO1S*PD1 were significantly different than the Av1nBg (+) control using a unpaired, t-test analysis, P value (0.024. When expressed as a percent of the Av1nBg control values, the influence of each mutation, individually or in combination, becomes apparent. The S* mutation dramatically reduced gene transfer to all four organs, whereas, the KO1 mutation did not. Thus, the importance of the shaft for transduction in vivo extends to organs besides the liver. Finally, gene transfer to the lung, heart, and kidney was diminished with PD1 suggesting a role for integrin binding in vector entry in these organs.

### EXAMPLE 16

Retargeting the S*, Shaft Modification and the 41sF Fiber In Vivo

Vectors containing the HSP mutation have been shown to effectively detarget adenoviral vectors in vivo (see examples 6 and 15). The objective of this study was to evaluate the ability to retarget vectors containing the S* modification or the Ad41sF to tumors in vivo. A cRGD peptide was genetically incorporated into the fiber HI loop and evaluated in vitro (Examples 8 and 14). These same vectors were then evaluated in vivo in tumor-bearing mice. Athymic nu/nu female mice were injected with 8×10⁶ A549 cells on the right hind flank. When tumors reached approximately 100 mm3 in size, they were randomized into treatment groups. Cohorts of 6 mice received each vector via tail vein injection at a dose of 1×10¹³ particles per kg. The animals were sacrificed approximately 72 hours after vector administration by carbon dioxide asphyxiation. Tumor, liver, heart, lung, spleen, and kidney were collected from each animal. Tissue from each organ was frozen to preserve it for real time PCR analysis to determine adenoviral hexon DNA content. Hexon real-time PCR was carried out as described in example 3. A separate sample of liver from each mouse was frozen to preserve it for a chemiluminescent β-galactosidase activity assay. Hexon real-time PCR and the chemiluminescent β-galactosidase activity assay was carried out as described in example 3.

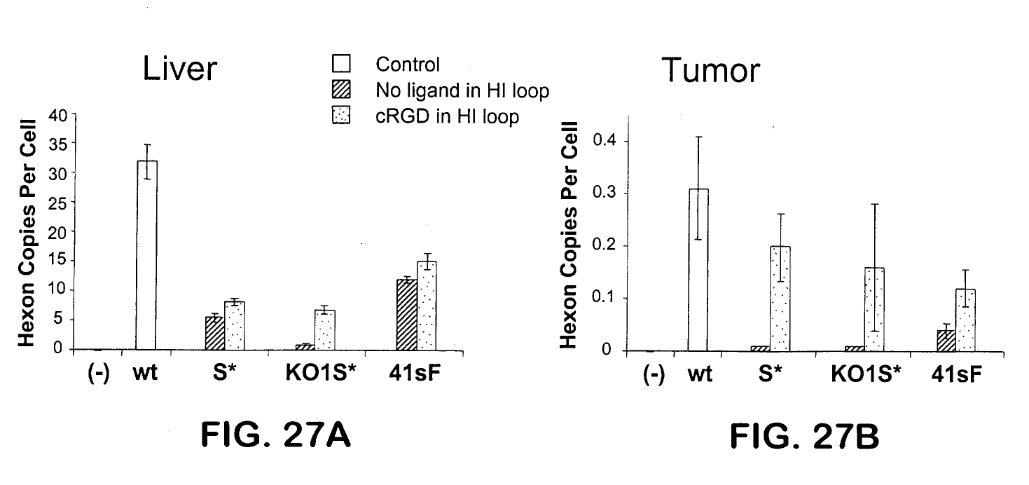

The adenoviral vector biodistribution to the liver and tumor for each treatment group is shown in FIG. 27. Vectors containing the S*, KO1S*, and 41sF fibers effectively detargeted the liver and tumor resulting in a significant reduction in the amount of adenoviral DNA found in each tissue in comparison to the Av1nBg control. Vectors containing the cRGD targeting ligand restored tranduction of the tumors to levels comparable to that achieved with the untargeted vector.

These data demonstrate successful liver detargeting accompanied with tumor retargeting. The extent of tumor retargeting is relates to the affinity and type of ligand that is used. These data demonstrate the successful development of a targeted, systemically deliverable adenoviral vector that will target tumors in vivo.

### EXAMPLE 17

Scale-Up Method for the Propagation of Detargeted Adenoviral Vectors

The growth and propagation of doubly or triply ablated adenoviral vectors requires novel scale up technologies. These detargeted vectors require alternative cellular entry strategies to allow for the efficient growth and generation of high titer preparations. A strategy for vector growth that is generally applicable to all detargeted adenoviral vectors, that does not require the development of new cell lines, and that aslo can be used for generating targeted vectors is provided herein.

Three recombinant adenoviral vectors were prepared that contain single mutations in the fiber or penton or both mutations combined into one vector. These vectors are designated Av3nBgFKO1, Av1nBgPD1, and Av1nBgFKO1PD1, respectively. The construction of these vectors is described above and a general description of each vector can be found in Table 1 above.

Scale-up of detargeted adenoviral vectors: A polycation, specifically hexadimethrine bromide was obtained from Sigma Chemical Co (St. Louis, Mo.), Catalog No. 52495, and was maintained in the medium at 4 μg/ml during the course of transfections and infections. To illustrate the affects of hexadimethrine bromide on the yield of detargeted adenoviral vectors the following experiment was carried out. Seven plates of AE1-2a adenoviral producer cells (Gorziglia et al (1996) J. Virology 70:4173-4178) were transduced with 10 particles per cells of each of the indicated vectors (See Table 4). Each vector was incubated with medium (Richters with 2% HI-FBS) containing hexadimethrine bromide at 4 μg/ml for 30 min at room temperature prior to infection. The infection was carried out for 2 hrs. Complete medium containing hexadimethrine bromide at 4 μg/ml was added to each plate. Final concentration of hexadimethrine bromide in all of-these experiments was maintained at 4 μg/ml. The titers were determined spectrophotometrically using the conversion of 10 D at A260 nm per 1×10¹² particles (Mittereder et al. (1996) J Virology 70:7498-7509). The total particle yield was then normalized for the number of plates used for transduction.

The inclusion of hexadimethrine bromide in the medium during the course of infection allows for the efficient propagation of detargeted adenoviral vectors containing fiber and penton mutations either alone or in combination. The affect of hexadimethrine bromide on vector yields is shown in Table 4. A 35-fold improvement in the yield of Av3nBgFKO1 was found when hexadimethrine bromide was included in the culture medium and resulted in increasing the vector yield from 1.3×10¹⁰ up to 4.6×10¹¹ vector particle per plate. Hexadimethrine bromide has a minimal effect on the yield of the Av1nBgPD1 adenoviral vector containing the penton, PD1 mutation with only a 1.2 fold improvement. The greatest effect using hexadimethrine bromide was found on the propagation of the doubly ablated adenoviral vector, Av1nBgFKO1PD1 with increases in vector yield from barely detectable levels up to 4.53×10¹⁰ vector particles per plate. These data demonstrate that use of nonspecific entry mechanisms allows for the efficient scale-up of detargeted adenoviral vectors.

TABLE 4
Efficient Scale-Up Of Detargeted Adenoviral Vectors Using
hexadimethrine bromide

| | Vector Yield (particles/plate) | | |
|---|---|---|---|
| | (−) hexadimethrine | (+) hexadimethrine | Fold |
| Vector | bromide | bromide | Improvement |
| Av1nBg | 3.89 × 10¹¹ | 5.72 × 10¹¹ | 1.47 |
| Av3nBg | 8.58 × 10¹⁰ | 2.38 × 10¹¹ | 2.77 |
| Av3nBgFKO1 | 1.30 × 10¹⁰ | 4.60 × 10¹¹ | 35.4 |
| Av1nBgPD1 | 1.95 × 10¹¹ | 2.40 × 10¹¹ | 1.23 |
| Av1nBg | TLTC* | 4.53 × 10¹⁰ | † |
| FKO1PD1 | | | |

*TLTC: Too low to count, a faint virus band was collected and the particle concentration was too dilute for titer determination.
†Significant improvement

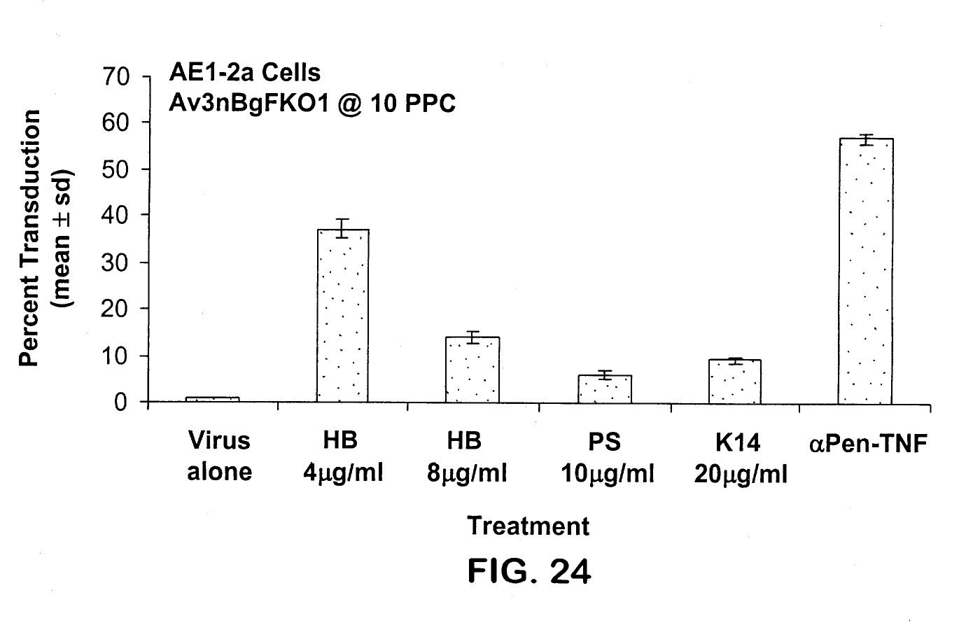

The use of alternative polycations including protamine sulfate and poly-lysine as well as bifunctional proteins such as the anti-penton:TNFα fusion protein was investigated. FIG. 24 show results that demonstrate all the reagents tested had some effect on enhancing transduction of the Av3nBgFKO1 vector. All of these compounds, when maintained in the medium during infection, enhanced transduction of the Av3nBgFKO1 detargeted adenoviral vector.

Bifunctional reagents: The use of bifunctional reagents for the propagation of detargeted adenoviral vectors was examined using the anti-penton:TNFα fusion protein. This particular reagent is a fusion protein between an antibody against Ad5 penton and the TNFα protein that is produced using stably transfected insect cells. This reagent will bind specifically to the adenoviral capsid via penton base and allow for binding to cell surface TNF receptors. The use of this reagent for the propagation of detargeted vectors is illustrated in Table 5 using Av3nBgFKO1 (also shown in FIG. 24). Monolayers of S8 cells were infected with 10 or 100 particles per cell of Av3nBgFKO1 or a control vector in the presence or absence of 1 ug/ml of the anti-penton:TNFα fusion protein. The monolayers were visually inspected over time for vector spread as indicated by the extent of cytopathic effect (CPE). The percentage of CPE at each time point is shown. The use of this bifunctional reagent clearly enhances the spread of the Av3nBgFKO1 vector throughout the monolayer.

TABLE 5
Efficient Scale-Up Of Detargeted Adenoviral
Vectors Using Bifunctional Reagents: Anti-Penton:TNFα

| | 10 ppc- | 10 ppc + | | |
|---|---|---|---|---|
| | anti-penton | anti-penton | 100 ppc − | 100 ppc + |
| | TNF | TNF | anti-penton TNF | anti-penton TNF |
| | Percentage of CPE | | | |
| Ad5Luc1 | | | | |
| 24 h | 0% | 0% | 0% | 0% |
| 48 h | 20-30% | 20-30% | 90-100% | 90-100% |
| 72 h | 60-70% | 80-90% | 100% | 100% |
| 120 h | 100% | 100% | 100% | 100% |
| Av3nBgKO1 24 hrs | | | | |
| 24 h | 0% | 0% | 0% | 0% |
| 48 h | 0% | 10-20% | 0% | 90-100% |
| 72 h | 5% | 60-70% | 5% | 100% |
| 120 h | 40-50% | 100% | 100% | 100% |

Since modifications will be apparent to those of skill in this art, it is intended that this invention be limited only by the scope of the appended claims.

What is claimed is:

1. A modified adenovirus capsid protein,
the unmodified capsid protein binds to heparin sulfate proteoglycan (HSP); and
the capsid protein comprises a mutation, whereby binding to heparin sulfate proteoglycan (HSP) is altered.

2. The modified protein of claim 1 that is a fiber protein

3. The capsid protein of claim 2, wherein the binding of the modified fiber protein is eliminated or reduced compared to the unmodified protein.

4. The modified protein of claim 2, wherein the binding of the modified fiber protein is eliminated or reduced compared to the unmodified protein.

5. The modified protein of claim 3 that comprises an insertion, deletion or replacement of amino acids.

6. The modified protein of claim 2, wherein the mutation alters the motif that binds to HSP, whereby HSP interaction is altered.

7. The modified protein of claim 6, motif is BBXB or BBBXXB, wherein the B is a basic amino acid and X is any amino acid.

8. The modified protein of claim 7, wherein the motif comprises the consensus sequence KKTK.

9. The modified protein of claim 2, wherein the fiber is a modified Ad5 or Ad2 fiber.

10. A modified protein of claim 2 that is a chimeric fiber protein, comprising portions of fiber proteins from at least two different adenoviruses, wherein:
a shaft or portion thereof is from a first adenovirus, whereby the resulting fiber does not bind to HSP or binds to HSP with reduced affinity compared to an unmodified fiber protein;
a shaft or portion thereof from the first adenovirus does not bind to HSP or binds to HSP with reduced affinity compared to the second adenovirus;
the second adenovirus binds to HSP; and
the portion comprises a sufficient portion to alter HSP binding of the resulting protein.

11. The modified protein of claim 10, wherein the binding to HSP of the modified fiber protein is eliminated or reduced compared to the unmodified protein.

12. The modified protein of claim 10, wherein the remainder of the fiber protein is from the second adenovirus.

13. The modified protein of claim 2, further comprising one or more further modifications that reduce or eliminate interaction of the resulting fiber with one or more cell surface proteins in addition to HSP.

14. The modified protein of claim 13, further comprising a ligand, whereby the resulting fiber binds to a receptor for the ligand.

15. The modified protein of claim 14, wherein the ligand is included in the knob region.

16. The modified protein of claim 14, wherein the ligand is inserted or it replaces a portion of the fiber, whereby the resulting fiber binds to a receptor for the ligand.

17. A modified protein of claim 11, wherein affinity for HSP is reduced at least by an amount selected from among reduced 5-fold, 10-fold and 100-fold.

18. The modified protein of claim 11, wherein the first adenovirus is selected from the group consisting of subgroup B, D or F, and the second is of subgroup C.

19. The modified protein of claim 10, wherein the first adenovirus is selected from the group consisting of Ad3, Ad35, Ad7, Ad11, Ad16, Ad21, Ad34, Ad40, Ad41 and Ad46.

20. The modified protein of claim 18, wherein the second adenovirus is Ad5 or Ad2.

21. The modified protein of claim 19, wherein the second adenovirus is Ad5 or Ad2.

22. A modified protein of claim 1 selected from the group consisting of a fiber protein comprising:
the sequence of amino acids set forth in any of SEQ ID Nos. 52, 54, 56, 58, 62, 66, 70 and 72; or
a sequence of amino acids having 90% sequence identity with a sequence of amino acids set forth in any of SEQ ID Nos. 52, 54, 56, 58, 62, 66, 70 and 72; or
a sequence of amino acids encoded by a sequence of nucleotides that hybridizes under conditions of high stringency along at least 70% of its length to a sequence of nucleotides that encodes a sequence of amino acids set forth in any of SEQ ID Nos. 52, 54, 56, 58, 62, 66, 70 and 72.

23. A nucleic acid molecule encoding a modified protein of any of claims 1-3, 10, 11, 13 and 14.

24. The nucleic acid molecule of claim 23 that comprises a vector.

25. The nucleic acid molecule of claim 24 that is an adenovirus vector.

26. The vector of claim 25 that is an adenoviral vector from a subgroup B, C or D adenovirus.

27. A cell, comprising a nucleic acid molecule of claim 23.

28. The cell of claim 27 that is a eukaryotic cell.

29. A cell, comprising a nucleic acid molecule of claim 25, wherein:
the cell is a eukaryotic cell; and
the cell in a packaging cell.

30. An adenoviral particle, comprising a modified protein of any of claims 1-3, 10, 11, 13 and 14, whereby binding of the viral particle to HSP is altered compared to a particle that expresses an unmodified fiber.

31. An adenoviral particle of claim 30, wherein a native receptor for the fiber is coxsackie-adenovirus receptor (CAR).

32. The adenoviral particle of claim 31, further comprising a mutation in the CAR-binding region of the capsid.

33. The adenoviral particle of claim 31, further comprising a mutation in the αᵥ integrin-binding region of the capsid, whereby binding to the integrin is eliminated or reduced.

34. The adenoviral particle of claim 32, further comprising a mutation in the αᵥ integrin-binding region of the capsid, whereby binding to the integrin is eliminated or reduced

35. The adenoviral particle of claim 31, wherein the CAR-binding region of the capsid modified is on a fiber knob.

36. The adenoviral particle of claim 35, wherein the fiber knob modification is in the AB loop or CD loop.

37. The adenoviral particle of claim 36, wherein the fiber knob modification is selected from the group consisting of KO1 and KO12.

38. The adenoviral particle of claim 32, wherein the adenovirus is a subgroup C, D or F adenovirus.

39. The adenoviral particle of claim 38, wherein the subgroup C virus is Ad2 or Ad5, the subgroup D virus is Ad46 and the subgroup F virus is Ad41.

40. The adenoviral vector of claim 25 that is an early generation adenoviral vector, a gutless adenoviral vector or a replication-conditional adenoviral vector.

41. The adenoviral vector of claim 40, wherein the replication-conditional adenoviral vector is an oncolytic adenoviral vector.

42. The adenoviral vector of claim 41, wherein the replication-conditional adenoviral vector is an oncolytic adenoviral vector.

43. The adenoviral vector of claim 25 that comprises heterologous nucleic acid.

44. The adenoviral vector of claim 43, wherein the heterologous nucleic acid encodes a polypeptide.

45. The adenoviral vector of claim 43, wherein the heterologous nucleic acid comprises or encodes a regulatory nucleic acid.

46. The adenoviral vector of claim 43, wherein the heterologous nucleic acid comprises or encodes a regulatory nucleic acid.

47. The adenoviral vector of claim 56, wherein the heterologous nucleic acid comprises or encodes a promoter or RNA.

48. The adenoviral vector of claim 47, wherein the promoter is a cell or tissue specific promoter.

49. The adenoviral vector of claim 47, wherein the promoter is operably linked to a gene of an adenovirus essential for replication.

50. The adenoviral vector of claim 48, wherein the tissue specific promoter is a tumor specific promoter.

51. The adenoviral vector of claim 44, wherein the polypeptide is a therapeutic polypeptide.

52. A method of expressing heterologous nucleic acid in a cell, comprising transducing the cell with an adenoviral vector of claim 44.

53. The method of claim 52, wherein:
the cell is a tumor cell;
the adenoviral vector is an oncolytic vector; and
the cell is killed.

54. The method of claim 52, wherein the cell is a mammalian cell.

55. The method of claim 54, wherein the cell is a primate cell.

56. The method of claim 55, wherein the cell is a human cell.

57. A method of reducing transduction of liver cells by an adenoviral particle, comprising reducing or eliminating binding of the particle to heparin sulfate proteoglycans (HSPs) on the liver cells.

58. A scale up method for the propagation of a detargeted adenoviral particle, comprising:
infecting a cell capable of replicating, maturing and packaging an adenoviral vector with a detargeted adenoviral vector in the presence of a reagent that results in entry of the adenoviral particle into the cell;
culturing the infected cell under conditions suitable for growth, spread and propagation of the adenoviral vector; and
recovering the resulting adenoviral particles.

59. The method of claim 58, wherein the reagent is a polycation.

60. The method of claim 59, wherein the polycation is selected from the group consisting of hexadimethrine bromide, polyethylenimine, protamine sulfate and poly-L-lysine.

61. The method of claim 58, wherein the reagent is a bifunctional protein that binds to the adenoviral particle and to a receptor on the cell.

62. The method of claim 61, wherein:
the bifunctional protein is selected from the group consisting of an anti-fiber antibody ligand fusion, an anti-fiber-Fab-FGF conjugate, an anti-penton-antibody ligand fusion, an anti-hexon antibody ligand fusion and a polylysine-peptide fusion, wherein the ligand is a ligand that binds to the receptor.

63. The method of claim 58, wherein the detargeted adenoviral particle expresses a modified capsid, whereby binding to at least one host cell receptor is reduced or eliminated compared with a wild-type adenovirus.

64. The method of claim 63, wherein the adenoviral particle is modified to eliminate or reduce binding with one host cell receptor.

65. The method of claim 63, wherein the adenoviral particle is modified to eliminate or reduce binding with two host cell receptors.

66. The method of claim 63, wherein the adenoviral particle is modified to eliminate or reduce binding with three host cell receptors.

67. The, method of claim 63, wherein the particle is modified with one or more mutations selected from the group consisting of mutations that reduce or eliminate interactions with one or more of αᵥ integrins, coxsackie-adenovirus receptors (CAR) and heparin sulfate proteoglycans (HSP).

68. The method of claim 67, wherein the mutation is selected from the group consisting of PD1, KO1, KO12 and S*.

69. The modified protein of claim 2, wherein the mutation is in the shaft of a fiber.

70. A modified protein of claim 3, wherein affinity for HSP is reduced at least by an amount selected from among reduced 5-fold, 10-fold and 100-fold.
